(19) United States
(12) Patent Application Publication
(10) Pub. No.: US 2009/0304620 A1
(43) Pub. Date: Dec. 10, 2009

(54) HAIR TREATMENT PRODUCTS COMPRISING POLYMERS

(75) Inventors: Erik Schulze zur Wiesche, Hamburg (DE); Volker Scheunemann, Luneburg (DE); Thomas Schröder, Hamburg (DE); Elisabeth Poppe, Hamburg (DE)

Correspondence Address: CONNOLLY BOVE LODGE & HUTZ LLP, PO BOX 2207, WILMINGTON, DE, 19899-2207, US

(73) Assignee: Henkel AG & Co. KGaA, Dusseldorf (DE)

(21) Appl. No.: 12/446,609

(22) PCT Filed: Sep. 20, 2007
(86) PCT No.: PCT/EP2007/059928

§ 371 Date: Apr. 22, 2009

(30) Foreign Application Priority Data
Oct. 24, 2006 (DE) 2006050650.2

(51) Int. Cl. A61K 8/72; A61Q 5/00

(52) U.S. Cl. 424/70.12; 424/70.11

(57) ABSTRACT

The invention relates to hair treatment products, comprising at least one copolymer made of 0.1 to 50% (in relation to the total number of monomers in the copolymer) monomers of the formula (I), wherein the unknowns are defined as in claim 1, and A2) are monomers from the group of acrylic acid, methacrylic acid and the like, and—optionally non-ionic monomers from the group of acrylamide, vinyl alcohol, and the like, wherein the monomers A2 and A3 together represent 50 to 99.9% (in relation to the total number of monomers in the copolymer) of the copolymer, at least one silicon and at least one selected care product, wherein the products result in advantageous effects for skin and hair.

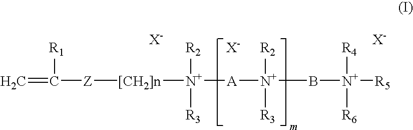

The present invention concerns cosmetic preparations, that is, cosmetic or dermatologic preparations to be applied to the skin, particularly human skin, and to be used on keratin fibers, particularly human hairs, and their use.

The compositions according to the invention are cosmetic agents. Although there was formerly a distinction between means for care of the human body and those for improving its appearance, these products are now defined jointly as “cosmetic agents” or cosmetic compositions.

Because of the general accessibility of cosmetic agents and their use on the human body, there is an extensive regulatory network to protect consumers in Germany and in the European Union. The legal basis in Germany is the Food and Necessities Act [Lebensmittel- und Bedarfsgegenstände-Gesetz (LBMG)] which defines cosmetic agents as follows in § 4:

“(1) Cosmetic agents in the sense of this Act are substances or compositions of substances intended to be used by humans externally or in the oral cavity for cleaning, care, or to influence appearance or body odor, or to convey an impression of odor, unless they are intended primarily to alleviate or eliminate diseases, pains, body injuries or diseases.” [§ 4 LBMG has not yet been brought into conformity with the EU cosmetics guideline, which speaks of six functions of cosmetics (cleaning, perfuming, altering appearance, influencing body odor, protecting, and maintaining in good condition)].
“(2) Substances or preparations of substances for cleaning or care of dental prostheses are equivalent to cosmetic agents.
“(3) Substances or preparations of substances intended to influence body forms are not cosmetic agents.”

A wide range of cosmetic agents are distinguished depending on the area of application, for example, those for skin care (bath preparations, skin washing and cleaning agents, skin care agents, eye cosmetics, lip care agents, nail care agents, intimate hygiene agents, foot care agents), those with particular actions (agents for protection against light, skin tanning agents, depigmenting agents, deodorants, antiperspirants, hair removal agents, shaving products, perfumes), those for dental or oral care (dental and oral care agents, denture care agents, denture adhesives) and those for hair care (shampoos, hair care agents, hair fixatives, hair shaping agents, agents for changing color).

Cosmetic compositions in the sense of the present invention are therefore selected, for example, from the groups of shower gels, shower baths, dental cleaning agents, mouth washes, hair shampoos, hair conditioners, conditioning shampoos, hair sprays, hair rinses, hair packs, hair tonics, permanent wave fixing solutions, hair-coloring shampoos, hair-coloring agents, hair fixatives, hair setting agents, hair styling preparations, Fönwell lotions, foam fixatives, hair gels, hair washes or combinations of them.

All of these agents need ingredients that provide more utility to the preparations, either with respect to applications technology, production technology, or in the opinion of the user, who will, for example, gain an improved subjective perception of the cosmetic agent because of certain ingredients. The main action of an ingredient can even differ, depending on the cosmetic agent. For instance, the exact same substance can have properties for caring for the hairs treated in a shampoo or a rinse, while in a rinse it may reduce staining of the skin or compensate for the action of the hair coloring agent, which may be aggressive.

In the field of cosmetics, then, there is always the desire to provide new substances for use that provide advantageous effects in individual product categories, and preferably in all of them.

In the area of care of keratinic fibers, especially, there is a continuous need for new care materials or combinations of care materials. In hair treatment agents, they are intended to result in improvement of the biophysical properties of the hair. The present invention is thus based particularly on the objective of providing hair treatment agents that strengthen the internal structure of the hair and reduce combing forces in wet and dry hair. In addition, the feel of the hair should be favorably affected.

It has now been found that a combination of selected care substances, silicones, and special polymers give hair treatment agents advantageous properties and, with those agents, care for, protect and strengthen the treated hairs in a special manner.

In a first embodiment, the subject of the invention is hair treatment agents comprising

A) at least one copolymer of
A1) 0.1% to 50% (based on the total number of monomers in the copolymer) monomers having the formula (I)

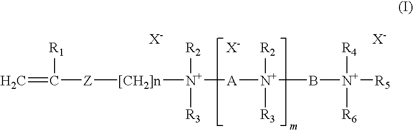

in which
   R¹ stands for —H or —CH₃ or —CH₂CH₃
   R², R³, R⁴, R⁵, R⁶ independently of each other, stand for —H or —CH₃ or —CH₂CH₃ or —(CH₂)₂CH₃ or —CH(CH₃)₂ or —(CH₂)₃CH₃ or —CH(CH₃)CH₂CH₃ or —CH₂—CH(CH₃)₂ or —C(CH₃)₂ or —CH₂—OH or —(CH₂)₂—OH or —CH(OH)—CH₃ or —CH(OH)—CH₂—CH₃ or —CH₂CH(OH)CH₃ or —(CH₂)₃OH or —CH₂—NH₂ or —CH₂—NH₂ or —CH(NH₂)—CH₃ or —CH(NH₂)CH₂CH₃ or —CH₂CH(NH₂)CH₃ or —(CH₂)₃NH₂
   n stands for values of 1 to 6, preferably for 2, 3, or 4
   m stands for values of 0 to 10, preferably for 0, 1 or 2
   Z stands for —O— or —C(O)—O— or —C(O)—NH—
   A stands for —CH₂— or —(CH₂)₂— or —(CH₂)₃— or —(CH₂)₄— or —(CH₂)₅— or —(CH₂)₆—
   B stands for a linear or branched C₂-C₁₂—, preferably C₃-C₁₀-polymethylene chain that may optionally be interrupted by one or more heteroatoms, especially by —O— or —NH—, and may optionally be substituted with one or more hydroxyl and/or amino groups, with the group B preferably standing for —(CH₂)₃— or —(CH₂)₄— or —(CH₂)₅— or —(CH₂)₆— or —CH₂CH(OH)CH₂— or —CH₂—CH(NH₂)—CH₂—
   X stands for an anion, preferably for chloride, bromide, iodide, sulfate or methosulfate,
A2) monomers from the group of acrylic acid, methacrylic acid, alpha-ethylacrylic acid, beta,beta-dimethylacrylic acid, methylenemalonic acid, vinylacetic acid, allylacetic acid, ethylideneacetic acid, propylideneacetic acid, crotonic acid, maleic acid, fumaric acid, itaconic acid, citraconic acid, methylfumaric acid, N-methacryloylalanine, N-acryloylhydroxyglycine, sulfopropyl acrylate, sulfoethyl acrylate, sulfoethyl methacrylate, styrene sulfonic acid, vinylsulfonic acid, vinylphosphonic acid, phosphoethyl acrylate, phosphonoethyl acrylate, phosphopropyl acrylate, phosphonopropyl acrylate, phosphoethyl methacrylate, phosphonoethyl methacrylate, phosphopropyl methacrylate and phosphonopropyl methacrylate, and the alkali metal and ammonium salts of those acids,
A3) optionally nonionic monomers from the group of acrylamide, vinyl alcohol, C₁-C₄-alkyl esters of acrylic acid and/or methacrylic acid, C₁-C₄-hydroxyalkyl esters of acrylic acid and/or methacrylic acid, especially ethylene glycol and propylene glycol acrylate and methacrylate, polyalkoxylated esters of acrylic acid and/or methacrylic acid, especially the polyethylene glycol and polypropylene glycol esters, esters of acrylic acid and/or methacrylic acid with polyethylene glycol or polypropylene glycol mono-(C₁-C₂₅)-alkyl ethers, vinyl acetate, vinylpyrrolidone and methylvinyl ether,
   in which the monomers A2 and A3, together, make up 50% to 99.9% (based on the total number of monomers in the copolymer) of the copolymer;

B) at least one silicone,

C) at least one care substance selected from
a) monomers, oligomers and polymers of amino acids, N—(C₂-C₂₄)-acylamino acids, the esters and/or the physiologically acceptable salts of these substances,
b) DNA or RNA oligonucleotides,
c) vitamins, provitamins or vitamin precursor stages of the groups A, B, C, E, H and K, and the esters of those substances,

d) α-hydroxycarboxylic acids, α-ketocarboxylic acids, β-hydroxycarboxylic acids and their esters, lactones or salt forms,
e) flavonoids and flavonoid-rich plant extracts,
f) isoflavonoids and isoflavonoid-rich plant extracts,
g) lyphenols and polyphenol-rich plant extracts,
h) ubiquinone and ubiquinol and their derivatives,
i) silymarin
j) ectoin
k) purine and/or purine derivatives,
l) carbohydrates, selected from monosaccharides, disaccharides and/or oligosaccharides,
m) hydantoin and/or hydantoin derivatives,
n) 2-furanone and/or 2-furanone derivatives
o) taurine (2-aminoethanesulfonic acid),
p) carnitine, carnitine tartrate, carnitine magnesium citrate, acetylcarnitine, 3-O-lauroyl-L-carnitine hydrochloride, 3-O-octanoyl-L-carnitine hydrochloride, 3-O-palmitoyl-L-carnitine hydrochloride, taurine, taurinlysylate, taurine tartrate, taurine ornithate, lysyltaurine and ornithyltaurine, betalaine, 1,1-dimethylproline, hercynine (Nα, Nα, Nα-trimethyl-L-histidinium betaine), ergothioneine (thioneine, 2-mercapto-Nα, Nα, Nα-trimethyl-L-histidinium betaine), choline, choline chloride, choline bitartrate, choline dihydrogen citrate,
q) and mixtures of these active ingredients a)-p).

Hairs treated with agents according to the invention exhibit improved elasticity and are more firmly fixed in their internal structure. That appears in, among other characteristics, in the higher measurements by differential thermal analysis and in improved wet and dry combabilities.

The agents according to the invention comprise, as the initial important ingredient, at least one copolymer of 0.1% to 50% (based on the total number of monomers in the copolymer) monomers having formula (I)

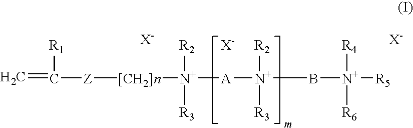

in which the previous definitions for the groups R¹, R², R³, R⁴, R⁵ and R⁶, as well as the indices n, m, and the groups Z, A, B, and X apply, and monomers of the group of acrylic acid, methacrylic acid, alpha-ethylacrylic acid, beta,beta-dimethylacrylic acid, methylenemalonic acid, vinylacetic acid, allylacetic acid, ethylideneacetic acid, propylideneacetic acid, crotonic acid, maleic acid, fumaric acid, itaconic acid, citraconic acid, mesaconic acid, N-methacryloylalanine, N-acryloylhydroxyglycine, sulfopropyl acrylate, sulfoethyl acrylate, sulfoethyl methacrylate, styrene sulfonic acid, vinylsulfonic acid, vinylphosphonic acid, phosphoethyl acrylate, phosphonoethyl acrylate, phosphopropyl acrylate, phosphonopropyl acrylate, phosphoethyl methacrylate, phosphonoethyl methacrylate, phosphopropyl methacrylate and phosphonopropyl methacrylate, and the alkali metal and ammonium salts of those acids, as well as optionally nonionic monomers from the group of acrylamide, vinyl alcohol, C₁-C₄-alkyl esters of acrylic acid and/or methacrylic acid, C₁-C₄-hydroxyalkyl esters of acrylic acid and/or methacrylic acid, especially ethylene glycol and propylene glycol acrylate and methacrylate, polyalkoxylated esters of acrylic acid and/or methacrylic acid, especially the polyethylene glycol and polypropylene glycol esters, esters of acrylic acid and/or methacrylic acid with polyethylene glycol or polypropylene glycol mono-(C₁-C₂₅)-alkyl ethers, vinyl acetate, vinylpyrrolidone and methylvinyl ether, in which the monomers A2 and A3 together make up 50% to 99.9% (based on the total number of monomers in the copolymer) of the copolymer.

In formula (I) for the monomer (A), R¹ is preferably a methyl group, and R², R³, R⁴, R⁵, and R⁶ also preferably stand for methyl groups. Group Z is preferably an —NH— group, and index n especially preferably stands for the number 3.

Various monomers having Formula (I) may be preferred, depending on the choice of groups A and B, and of the index m. A preferred monomer that meets the criteria stated in the previous paragraph also has, as group B, a —CH₂—CH(OH)—CH₂— group, and index m stands for the number 0. Polymers that comprise such monomers are used preferably within narrow quantitative ranges. Hair treatment agents according to the invention are characterized by comprising, based on its weight, 0.001 to 5% by weight, preferably 0.0025 to 2.5% by weight, and especially preferably 0.005 to 1% by weight, further preferably 0.0075 to 0.75% by weight, and especially 0.01 to 0.5% by weight of at least one copolymer A of

A1) 0.1 to 50%, preferably 10 to 50% (based on the total number of monomers in the copolymer) of monomers having the formula (Ia)

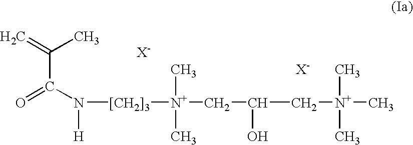

in which
X stands for chloride, sulfate, or methosulfate,
   A2) monomers of the group acrylic acid, methacrylic acid, and the alkali metal and ammonium salts of those acids,
   in which the monomers A2 make up 50 to 99.9%, preferably 50 to 90% (based on the total number of monomers in the copolymer) of the copolymer.

Another preferred monomer that meets the criteria stated in the previous paragraph has, as group B, also a —CH₂—CH(OH)—CH₂— group, as group A a —(CH₂)₂— group, a —(CH₂)₃, or a —(CH₂)₄— group, and the index m stands for the number 1. Polymers that comprise such monomers are also used preferably within a narrow quantitative range. In this way, hair treatment agents preferred according to the invention are distinguished by having, based on its weight, 0.001 to 5% by weight, preferably 0.0025 to 2.5% by weight, and especially preferably 0.005 to 1% by weight, further preferably 0.0075 to 0.75% by weight, and especially 0.01 to 0.5,% by weight of at least one copolymer A of

A1) 0.1 to 50%, preferably 10 to 50% (based on the total number of monomers in the copolymer) monomers having formula (Ib)

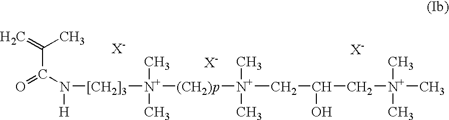

in which
p stands for 2, 3 or 4
X stands for chloride, sulfate or methosulfate,
   A2) monomers of the group acrylic acid, methacrylic acid, and the alkali metal and ammonium salts of those acids,
   with the monomers A2 making up 50 to 99.9%, preferably 50 to 90% (based on the total number of monomers in the copolymer) of the copolymer.

Particularly preferred monomers A2 are acrylic acid or its salts (including mixtures, i.e., partially neutralized acrylic acids) and acrylamide. One preferred copolymer A is a copolymer of the monomer (Ia), sodium acrylate and acrylamide, in which the following distribution (in percentages of the total monomers contained in the polymer):

Monomer (Ia): 0.1 to 50%, preferably 10 to 50%

Sodium acrylate: 10 to 95%, preferably 50 to 70%

Acrylamide: 0 to 50%, preferably 0 to 30%.

A preferred copolymer A preferably contains the following numbers of the different monomers:
Monomer (Ia): Values of 1 to 12,500, preferably 2 to 8,000, especially preferably 3 to 4,000 and particularly 5 to 2000
Sodium acrylate: Values of 1 to 24,000, preferably 5 to 15,000, especially preferably 10 to 10,000 and particularly 100 to 4,800
Acrylamide: Values of 0 1, 2, 3, 4, or 5, with the value of 0 preferred.

To summarize, hair treatment agents according to the invention are preferred which comprise, based on its weight, 0.001 to 5% by weight, preferably 0.0025 to 2.5% by weight, and especially preferably 0.005 to 1% by weight, further preferably 0.0075 to 0.75% by weight, and especially 0.01 to 0.5% by weight of at least one copolymer A having the general formula (Ic)

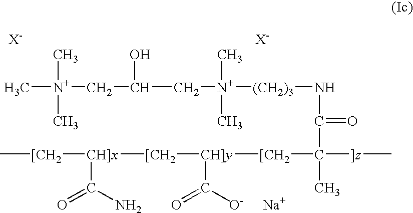

in which

x+y+z=Q
Q stands for values of 3 to 55,000, preferably of 10 to 25,000, especially preferably 50 to 15,000, further preferably 100 to 10,000, still further preferably 500 to 8,000, and particularly 1,000 to 5,000
x stands for (0 to 0.5) Q, preferably for (0 to 0.3) Q, and especially for the values 0, 1, 2, 3, 4, 5, with value 0 preferred,
y stands for (0.1 to 0.95) Q, preferably for (0.5 to 0.7) Q, and particularly for values of 1 to 24,000, preferably 5 to 15,000, especially preferably 10 to 10,000, and particularly 100 to 4,800.
z stands for (0.001 to 0.5) Q, preferably for (0.1 to 0.5) Q, and especially for values of 1 to 12,500, preferably 2 to 8,000, especially preferably 3 to 4,000 and particularly 5 to 2,000.

In addition to, or in place of, the copolymers named above, the agents according to the invention may also comprise a copolymer built up of monomers of formula (Ia). maleic or fumaric acids (or their disodium salts) and acrylamide. Here the following distribution (in percent of the total monomers contained in the polymer) is preferred:
Monomer (Ia): 0.1 to 50%, preferably 10 to 50%
Maleic or fumaric acid (or their disodium salts): 10 to 95%, preferably 50 to 70%
Acrylamide: 0 to 50%, preferably 0 to 30%

A preferred copolymer A preferably comprises the following numbers of the particular monomers:
Monomer (Ia): Values of 1 to 12,500, preferably 2 to 8,000, especially preferably 3 to 4,000, and particularly 5 to
   Maleic or fumaric acid (or their disodium salts): Values of 1 to 24,000, preferably 5 to 15,000, especially preferably 10 to 10,000, and particularly 100 to 4,800
Acrylamide: Values 0, 1, 2, 3, 4, or 5, with the value 0 preferred.

To summarize, hair treatment agents according to the invention are preferred which comprise, based on its weight, 0.001 to 5% by weight, preferably 0.0025 to 2.5% by weight, and especially preferably 0.005 to 1% by weight, further preferably 0.0075 to 0.75% by weight, and especially 0.01 to 0.5% by weight of at least one copolymer A having the general formula (Id)

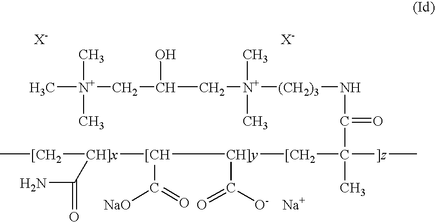

in which

x+y+z=Q
Q stands for values of 3 to 55,000, preferably of 10 to 25,000, especially preferably 50 to 15,000, further preferably 100 to 10,000, still further preferably 500 to 8,000, and particularly 1,000 to 5,000,
x stands for (0 to 0.5) Q, preferably for (0 to 0.3) Q, and especially for the values 0, 1, 2, 3, 4, 5, with value 0 preferred,
y stands for (0.1 to 0.95) Q, preferably for (0.5 to 0.7) Q, and particularly for values of 1 to 24,000, preferably 5 to 15,000, especially preferably 10 to 10,000, and particularly 100 to 4,800,
z stands for (0.001 to 0.5) Q, preferably for (0.1 to 0.5) Q, and especially for values of 1 to 12,500, preferably 2 to 8,000, especially preferably 3 to 4,000 and particularly 5 to 2,000.

In addition to, or in place of, the copolymers named above, the agents according to the invention may also comprise a copolymer built up of monomers of formula (Ia). vinyl sulfonic acid (or its sodium salt) and acrylamide. Here the following distribution (in percent of the total monomers contained in the polymer) is preferred:
Monomer (Ia): 0.1 to 50%, preferably 10 to 50%
Sodium vinylsulfonate: 10 to 95%, preferably 50 to 70%
Acrylamide: 0 to 50%, preferably 0 to 30%

A preferred copolymer A preferably comprises the following numbers of the particular monomers:
Monomer (Ia): Values of 1 to 12,500, preferably 2 to 8,000, especially preferably 3 to 4,000, and particularly 5 to 2,000
Sodium vinylsulfonate: Values of 1 to 24,000, preferably 5 to 15,000, especially preferably 10 to 10,000, and particularly 100 to 4,800
Acrylamide: Values 0, 1, 2, 3, 4, or 5, with the value 0 preferred.

To summarize, hair treatment agents according to the invention are preferred which comprise, based on its weight, 0.001 to 5% by weight, preferably 0.0025 to 2.5% by weight, and especially preferably 0.005 to 1% by weight, further preferably 0.0075 to 0.75% by weight, and especially 0.01 to 0.5% by weight of at least one copolymer A having the general formula (Ie)

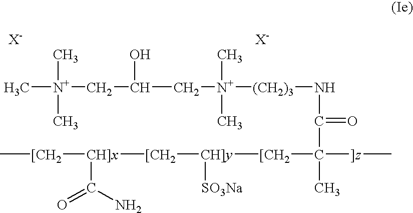

in which

x+y+z=Q
Q stands for values of 3 to 55,000, preferably of 10 to 25,000, especially preferably 50 to 15,000, further preferably 100 to 10,000, still further preferably 500 to 8,000, and particularly 1,000 to 5,000
x stands for (0 to 0.5) Q, preferably for (0 to 0.3) Q, and especially for the values 0, 1, 2, 3, 4, 5, with value 0 preferred,
y stands for (0.1 to 0.95) Q, preferably for (0.5 to 0.7) Q, and particularly for values of 1 to 24,000, preferably 5 to 15,000, especially preferably 10 to 10,000, and particularly 100 to 4,800,
z stands for (0.001 to 0.5) Q, preferably for (0.1 to 0.5) Q, and especially for values of 1 to 12,500, preferably 2 to 8,000, especially preferably 3 to 4,000 and particularly 5 to 2,000.

In addition to, or in place of, the copolymers named above, the agents according to the invention may also comprise a copolymer built up of monomers of formula (Ia). styrene sulfonic acid (or its sodium salt) and acrylamide. Here the following distribution (in percent of the total monomers contained in the polymer) is preferred:
Monomer (Ia): 0.1 to 50%, preferably 10 to 50%
Sodium styrenesulfonate: 10 to 95%, preferably 50 to 70%
Acrylamide: 0 to 50%, preferably 0 to 30%

A preferred copolymer A preferably comprises the following numbers of the particular monomers:
Monomer (Ia): Values of 1 to 12,500, preferably 2 to 8,000, especially preferably 3 to 4,000, and particularly 5 to 2,000
Sodium styrenesulfonate: Values of 1 to 24,000, preferably 5 to 15,000, especially preferably 10 to 10,000, and particularly 100 to 4,800
Acrylamide: Values 0, 1, 2, 3, 4, or 5, with the value 0 preferred.

To summarize, hair treatment agents according to the invention are preferred which comprise, based on its weight, 0.001 to 5% by weight, preferably 0.0025 to 2.5% by weight, and especially preferably 0.005 to 1% by weight, further preferably 0.0075 to 0.75% by weight, and especially 0.01 to 0.5% by weight of at least one copolymer A having the general formula (If)

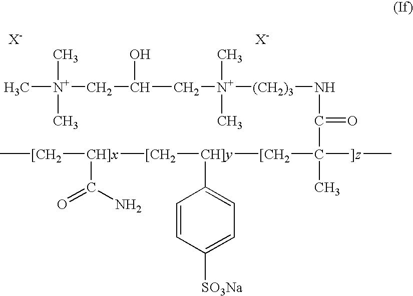

in which

x+y+z=Q
Q stands for values of 3 to 55,000, preferably of 10 to 25,000, especially preferably 50 to 15,000, further preferably 100 to 10,000, still further preferably 500 to 8,000, and particularly 1,000 to 5,000,
x stands for (0 to 0.5) Q, preferably for (0 to 0.3) Q, and especially for the values 0, 1, 2, 3, 4, 5, with value 0 preferred,
y stands for (0.1 to 0.95) Q, preferably for (0.5 to 0.7) Q, and particularly for values of 1 to 24,000, preferably 5 to 15,000, especially preferably 10 to 10,000, and particularly 100 to 4,800,
z stands for (0.001 to 0.5) Q, preferably for (0.1 to 0.5) Q, and especially for values of 1 to 12,500, preferably 2 to 8,000, especially preferably 3 to 4,000 and particularly 5 to 2,000.

Independently of which of the preferred copolymers A having formulas (Ic) to (If) are used, those hair treatment agents according to the invention are preferred that are distinguished by the ratio of (y:z) of 4:1 to 1:2, preferably 4:1 to 1:1.

Independently of which of the preferred copolymers A having formulas (Ic) to (If) are use, those hair treatment agents according to the invention are preferred in which copolymer A has a molecular weight of 10,000 to 20 million g/mol, preferably 100,000 to 10 million g/mol, further preferably 500,000 to 5 million g/mol and particularly 1.1 million to 2.2 million g/mol¹.

As the second essential component the agents according to the invention comprise at least one silicone. It is preferred to use silicones having the formula Si-I

(CH₃)₃Si—[O—Si(CH₃)₂]ₓ—O—Si(CH₃)₃ (Si-I),

in which x stands for a number from 0 to 5,000, preferably from 10 to 2,500, further preferably from 50 to 1,500 and particularly 100 to 1000.

These simple linear silicone polymers are built up according to the plan (R₂SiO)ₓ. Such silicones are designated systematically as polyorganosiloxanes. This nomenclature is based on calling the Si—O—Si bond a siloxane bond, and is established in the scientific literature. A polymer having the general formula Si-I is called poly(dimethylsiloxane), but according to the IUPAC rules for naming linear organic polymers it can also be called poly[oxy(dimethylsilylene)]. According to the rules for inorganic macromolecules, it can be called catena-poly[(dimethylsilicon)-μ-oxo]. The international generic name of the compounds is dimethicone, and they are called dimethicones according to INCI.

Agents preferred according to the invention comprise dimethicones, i.e., silicones having the formula Si-I, in narrow quantitative ranges. Here, hair treatment agents according to the invention are preferred, which comprise, on a weight basis, 0.01 to 5% by weight, preferably 0.025 to 2.5% by weight, especially preferably 0.05 to 1% by weight, further preferably 0.075 to 0.75% by weight, and particularly 0.1 to 0.25% by weight of at least one silicone of the formula Si-I

(CH₃)₃Si—[O—Si(CH₃)₂]ₓ—O—Si(CH₃)₃ (Si-I),

in which x stands for a number from 0 to 5,000, preferably from 10 to 2,500, further preferably 50 to 1,500 and particularly 100 to 1,000.

The silicones of the formula Si-I can have degrees of oligomerization or polymerization from 0 (equivalent to (CH₃)₃Si—O—Si(CH₃)₃) to 500, with the preferred degrees of oligomerization or polymerization being in the range of 10 to 2,500, further preferably from 50 to 1,500 and particularly from 100 to 1,000. Of course, the molecular weight of the dimethicone varies with the degree of oligomerization or polymerization, being between 162 Dalton (for the 0 degree of oligomerization stated above) and some hundreds of kDa for high degrees of polymerization, such as about 60 kDa for a polymerization degree of 810.

The viscosities of the dimethicones used according to the invention are nearly independent of the temperature and vary with the molecular weight, with dimethicones of higher molecular weight exhibiting higher viscosities than those of lower molecular weight. Under the usual conditions of measurement (10° C., 1013.25 mbar) (CH₃)₃Si—O—Si(CH₃)₃ has a viscosity of 0.65 mm²/sec (centistokes, cSt). This viscosity also has hardly any dependence on the equipment used for the measurement. For example, the viscosity can be measured under the normal measurement conditions stated above with a Brookfield LVT Viscosimeter, spindle 2, 30 rpm. Viscosities reported in the present application are always based on 20° C. and 1013.25 mbar and on equipment with which a viscosity of 065 mm²/sec (centistokes, cSt) is determined for CH₃)₃Si—O—Si(CH₃)₃.

Particularly preferred hair treatment agents according to the invention are distinguished by containing, as the silicone of formula Si-I, a dimethicone having a viscosity of 10 to 1,000,000 cSt, preferably 100 to 950,000 cSt, especially preferably 1,000 to 900,000 cSt, further preferably 5,000 to 850,000 cSt and particularly 100,000 to 800,000 cSt (measured at 20° C.).

Dimethicones that can be used with particular preference according to the invention have degrees of polymerization between 700 and 1,400. Especially preferred hair treatment agents according to the invention are distinguished by at least 25% by weight, preferably at least 50% by weight, especially preferably at least 75% by weight, further preferably at least 95% by weight and particularly 100% by weight of the silicones of formula Si-I comprised in the agent being selected from silicones of the formula Si-I

(CH₃)₃Si—[O—Si(CH₃)₂]ₓ—O—Si(CH₃)₃ (Si-I),

in which x stands for a number from the group 800, 801, 802, 803, 804, 805, 806, 807, 808, 809, 810, 811, 812, 813, 814, 815, 816, 817, 818, 819, 820, 821, 822, 823, 824, 825, 825, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100

Agents that are especially preferred according to the invention thus comprise silicones of the formula Si-I, selected from the dimethicones (CH₃)₃Si—[O—Si(CH₃)₂]₈₀₀—O—Si(CH₃)₃, (CH₃)₃Si—[O—Si(CH₃)₂]₈₀₁—O—Si(CH₃)₃, (CH₃)₃Si—[O—Si(CH₃)₂]₈₀₂—O—Si(CH₃)₃, (CH₃)₃Si—[O—Si(CH₃)₂]₈₀₃—O—Si(CH₃)₃, (CH₃)₃Si—[O—Si(CH₃)₂]₈₀₄—O—Si(CH₃)₃, (CH₃)₃Si—[O—Si(CH₃)₂]₈₀₅—O—Si(CH₃)₃, (CH₃)₃Si—[O—Si(CH₃)₂]₈₀₆—O—Si(CH₃)₃, (CH₃)₃Si—[O—Si(CH₃)₂]₈₀₇—O—Si(CH₃)₃, (CH₃)₃Si—[O—Si(CH₃)₂]₈₀₈—O—Si(CH₃)₃, (CH₃)₃Si—[O—Si(CH₃)₂]₈₀₉—O—Si(CH₃)₃, (CH₃)₃Si—[O—Si(CH₃)₂]₈₁₀—O—Si(CH₃)₃, (CH₃)₃Si—[O—Si(CH₃)₂]₈₁₁—O—Si(CH₃)₃, (CH₃)₃Si—[O—Si(CH₃)₂]₈₁₂—O—Si(CH₃)₃, (CH₃)₃Si—[O—Si(CH₃)₂]₈₁₃—O—Si(CH₃)₃, (CH₃)₃Si—[O—Si(CH₃)₂]₈₁₄—O—Si(CH₃)₃, (CH₃)₃Si—[O—Si(CH₃)₂]₈₁₅—O—Si(CH₃)₃, (CH₃)₃Si—[O—Si(CH₃)₂]⁸¹⁶—O—Si(CH₃)₃, (CH₃)₃Si—[O—Si(CH₃)₂]₈₁₇—O—Si(CH₃)₃, (CH₃)₃Si—[O—Si(CH₃)₂]₈₁₈—O—Si(CH₃)₃, (CH₃)₃Si—[O—Si(CH₃)₂]₈₁₉—O—Si(CH₃)₃, (CH₃)₃Si—[O—Si(CH₃)₂]₈₂₀—O—Si(CH₃)₃, (CH₃)₃Si—[O—Si(CH₃)₂]₈₂₁—O—Si(CH₃)₃, (CH₃)₃Si—[O—Si(CH₃)₂]₈₂₂—O—Si(CH₃)₃, (CH₃)₃Si—[O—Si(CH₃)₂]₈₂₃—O—Si(CH₃)₃, (CH₃)₃Si—[O—Si(CH₃)₂]₈₂₄—O—Si(CH₃)₃, (CH₃)₃Si—[O—Si(CH₃)₂]₈₂₅—O—Si(CH₃)₃, (CH₃)₃Si—[O—Si(CH₃)₂]₈₂₆—O—Si(CH₃)₃, (CH₃)₃Si—[O—Si(CH₃)₂]₈₂₇—O—Si(CH₃)₃, (CH₃)₃Si—[O—Si(CH₃)₂]₈₂₈—O—Si(CH₃)₃, (CH₃)₃Si—[O—Si(CH₃)₂]₈₂₉—O—Si(CH₃)₃, (CH₃)₃Si—[O—Si(CH₃)₂]₈₃₀—O—Si(CH₃)₃, (CH₃)₃Si—[O—Si(CH₃)₂]₈₃₁—O—Si(CH₃)₃, (CH₃)₃Si—[O—Si(CH₃)₂]₈₃₂—O—Si(CH₃)₃, (CH₃)₃Si—[O—Si(CH₃)₂]₈₃₃—O—Si(CH₃)₃, (CH₃)₃Si—[O—Si(CH₃)₂]₈₃₄—O—Si(CH₃)₃, (CH₃)₃Si—[O—Si(CH₃)₂]₈₃₅—O—Si(CH₃)₃, (CH₃)₃Si—[O—Si(CH₃)₂]₈₃₆—O—Si(CH₃)₃, (CH₃)₃Si—[O—Si(CH₃)₂]₈₃₇—O—Si(CH₃)₃, (CH₃)₃Si—[O—Si(CH₃)₂]₈₃₈—O—Si(CH₃)₃, (CH₃)₃Si—[O—Si(CH₃)₂]₈₃₉—O—Si(CH₃)₃, (CH₃)₃Si—[O—Si(CH₃)₂]₈₄₀—O—Si(CH₃)₃, (CH₃)₃Si—[O—Si(CH₃)₂]₈₄₁—O—Si(CH₃)₃, (CH₃)₃Si—[O—Si(CH₃)₂]₈₄₂—O—Si(CH₃)₃, (CH₃)₃Si—[O—Si(CH₃)₂]₈₄₃—O—Si(CH₃)₃, (CH₃)₃Si—[O—Si(CH₃)₂]₈₄₄—O—Si(CH₃)₃, (CH₃)₃Si—[O—Si(CH₃)₂]₈₄₅—O—Si(CH₃)₃, (CH₃)₃Si—[O—Si(CH₃)₂]₈₄₆—O—Si(CH₃)₃, (CH₃)₃Si—[O—Si(CH₃)₂]₈₄₇—O—Si(CH₃)₃, (CH₃)₃Si—[O—Si(CH₃)₂]₈₄₈—O—Si(CH₃)₃, (CH₃)₃Si—[O—Si(CH₃)₂]₈₄₉—O—Si(CH₃)₃, (CH₃)₃Si—[O—Si(CH₃)₂]₈₅₀—O—Si(CH₃)₃, (CH₃)₃Si—[O—Si(CH₃)₂]₈₅₁—O—Si(CH₃)₃, (CH₃)₃Si—[O—Si(CH₃)₂]₈₅₂—O—Si(CH₃)₃, (CH₃)₃Si—[O—Si(CH₃)₂]₈₅₃—O—Si(CH₃)₃, (CH₃)₃Si—[O—Si(CH₃)₂]₈₅₄—O—Si(CH₃)₃, (CH₃)₃Si—[O—Si(CH₃)₂]₈₅₅—O—Si(CH₃)₃, (CH₃)₃Si—[O—Si(CH₃)₂]₈₅₆—O—Si(CH₃)₃, (CH₃)₃Si—[O—Si(CH₃)₂]₈₅₇—O—Si(CH₃)₃, (CH₃)₃Si—[O—Si(CH₃)₂]₈₅₈—O—Si(CH₃)₃, (CH₃)₃Si—[O—Si(CH₃)₂]₈₅₉—O—Si(CH₃)₃, (CH₃)₃Si—[O—Si(CH₃)₂]₈₆₀—O—Si(CH₃)₃, (CH₃)₃Si—[O—Si(CH₃)₂]₈₆₁—O—Si(CH₃)₃, (CH₃)₃Si—[O—Si(CH₃)₂]₈₆₂—O—Si(CH₃)₃, (CH₃)₃Si—[O—Si(CH₃)₂]₈₆₃—O—Si(CH₃)₃, (CH₃)₃Si—[O—Si(CH₃)₂]₈₆₄—O—Si(CH₃)₃, (CH₃)₃Si—[O—Si(CH₃)₂]₈₆₅—O—Si(CH₃)₃, (CH₃)₃Si—[O—Si(CH₃)₂]₈₆₆—O—Si(CH₃)₃, (CH₃)₃Si—[O—Si(CH₃)₂]₈₆₇—O—Si(CH₃)₃, (CH₃)₃Si—[O—Si(CH₃)₂]₈₆₈—O—Si(CH₃)₃, (CH₃)₃Si—[O—Si(CH₃)₂]₈₆₉—O—Si(CH₃)₃, (CH₃)₃Si—[O—Si(CH₃)₂]₈₇₀—O—Si(CH₃)₃, (CH₃)₃Si—[O—Si(CH₃)₂]₈₇₁—O—Si(CH₃)₃, (CH₃)₃Si—[O—Si(CH₃)₂]₈₇₂—O—Si(CH₃)₃, (CH₃)₃Si—[O—Si(CH₃)₂]₈₇₃—O—Si(CH₃)₃, (CH₃)₃Si—[O—Si(CH₃)₂]₈₇₄—O—Si(CH₃)₃, (CH₃)₃Si—[O—Si(CH₃)₂]₈₇₅—O—Si(CH₃)₃, (CH₃)₃Si—[O—Si(CH₃)₂]₈₇₆—O—Si(CH₃)₃, (CH₃)₃Si—[O—Si(CH₃)₂]₈₇₇—O—Si(CH₃)₃, (CH₃)₃Si—[O—Si(CH₃)₂]₈₇₈—O—Si(CH₃)₃, (CH₃)₃Si—[O—Si(CH₃)₂]₈₇₉—O—Si(CH₃)₃, (CH₃)₃Si—[O—Si(CH₃)₂]₈₈₀—O—Si(CH₃)₃, (CH₃)₃Si—[O—Si(CH₃)₂]₈₈₁—O—Si(CH₃)₃, (CH₃)₃Si—[O—Si(CH₃)₂]₈₈₂—O—Si(CH₃)₃, (CH₃)₃Si—[O—Si(CH₃)₂]₈₈₃—O—Si(CH₃)₃, (CH₃)₃Si—[O—Si(CH₃)₂]₈₈₄—O—Si(CH₃)₃, (CH₃)₃Si—[O—Si(CH₃)₂]₈₈₅—O—Si(CH₃)₃, (CH₃)₃Si—[O—Si(CH₃)₂]₈₈₆—O—Si(CH₃)₃, (CH₃)₃Si—[O—Si(CH₃)₂]₈₈₇—O—Si(CH₃)₃, (CH₃)₃Si—[O—Si(CH₃)₂]₈₈₈—O—Si(CH₃)₃, (CH₃)₃Si—[O—Si(CH₃)₂]₈₈₉—O—Si(CH₃)₃, (CH₃)₃Si—[O—Si(CH₃)₂]₈₉₀—O—Si(CH₃)₃, (CH₃)₃Si—[O—Si(CH₃)₂]₈₉₁—O—Si(CH₃)₃, (CH₃)₃Si—[O—Si(CH₃)₂]₈₉₂—O—Si(CH₃)₃, (CH₃)₃Si—[O—Si(CH₃)₂]₈₉₃—O—Si(CH₃)₃, (CH₃)₃Si—[O—Si(CH₃)₂]₈₉₄—O—Si(CH₃)₃, (CH₃)₃Si—[O—Si(CH₃)₂]₈₉₅—O—Si(CH₃)₃, (CH₃)₃Si—[O—Si(CH₃)₂]₈₉₆—O—Si(CH₃)₃, (CH₃)₃Si—[O—Si(CH₃)₂]₈₉₇—O—Si(CH₃)₃, (CH₃)₃Si—[O—Si(CH₃)₂]₈₉₈—O—Si(CH₃)₃, (CH₃)₃Si—[O—Si(CH₃)₂]₈₉₉—O—Si(CH₃)₃, (CH₃)₃Si—[O—Si(CH₃)₂]₉₀₀—O—Si(CH₃)₃, (CH₃)₃Si—[O—Si(CH₃)₂]₉₀₁—O—Si(CH₃)₃, (CH₃)₃Si—[O—Si(CH₃)₂]₉₀₂—O—Si(CH₃)₃, (CH₃)₃Si—[O—Si(CH₃)₂]₉₀₃—O—Si(CH₃)₃, (CH₃)₃Si—[O—Si(CH₃)₂]₉₀₄—O—Si(CH₃)₃, (CH₃)₃Si—[O—Si(CH₃)₂]₉₀₅—O—Si(CH₃)₃, (CH₃)₃Si—[O—Si(CH₃)₂]₉₀₆—O—Si(CH₃)₃, (CH₃)₃Si—[O—Si(CH₃)₂]₉₀₇—O—Si(CH₃)₃, (CH₃)₃Si—[O—Si(CH₃)₂]₉₀₈—O—Si(CH₃)₃, (CH₃)₃Si—[O—Si(CH₃)₂]₉₀₉—O—Si(CH₃)₃, (CH₃)₃Si—[O—Si(CH₃)₂]₉₁₀—O—Si(CH₃)₃, (CH₃)₃Si—[O—Si(CH₃)₂]₉₁₁—O—Si(CH₃)₃, (CH₃)₃Si—[O—Si(CH₃)₂]₉₁₂—O—Si(CH₃)₃, (CH₃)₃Si—[O—Si(CH₃)₂]₉₁₃—O—Si(CH₃)₃, (CH₃)₃Si—[O—Si(CH₃)₂]₉₁₄—O—Si(CH₃)₃, (CH₃)₃Si—[O—Si(CH₃)₂]₉₁₅—O—Si(CH₃)₃, (CH₃)₃Si—[O—Si(CH₃)₂]₉₁₆—O—Si(CH₃)₃, (CH₃)₃Si—[O—Si(CH₃)₂]₉₁₇—O—Si(CH₃)₃, (CH₃)₃Si—[O—Si(CH₃)₂]₉₁₈—O—Si(CH₃)₃, (CH₃)₃Si—[O—Si(CH₃)₂]₉₁₉—O—Si(CH₃)₃, (CH₃)₃Si—[O—Si(CH₃)₂]₉₂₀—O—Si(CH₃)₃, (CH₃)₃Si—[O—Si(CH₃)₂]₉₂₁—O—Si(CH₃)₃, (CH₃)₃Si—[O—Si(CH₃)₂]₉₂₂—O—Si(CH₃)₃, (CH₃)₃Si—[O—Si(CH₃)₂]₉₂₃—O—Si(CH₃)₃, (CH₃)₃Si—[O—Si(CH₃)₂]₉₂₄—O—Si(CH₃)₃, (CH₃)₃Si—[O—Si(CH₃)₂]₉₂₅—O—Si(CH₃)₃, (CH₃)₃Si—[O—Si(CH₃)₂]₉₂₆—O—Si(CH₃)₃, (CH₃)₃Si—[O—Si(CH₃)₂]₉₂₇—O—Si(CH₃)₃, (CH₃)₃Si—[O—Si(CH₃)₂]₉₂₈—O—Si(CH₃)₃, (CH₃)₃Si—[O—Si(CH₃)₂]₉₂₉—O—Si(CH₃)₃, (CH₃)₃Si—[O—Si(CH₃)₂]₉₃₀—O—Si(CH₃)₃, (CH₃)₃Si—[O—Si(CH₃)₂]₉₃₁—O—Si(CH₃)₃, (CH₃)₃Si—[O—Si(CH₃)₂]₉₃₂—O—Si(CH₃)₃, (CH₃)₃Si—[O—Si(CH₃)₂]₉₃₃—O—Si(CH₃)₃, (CH₃)₃Si—[O—Si(CH₃)₂]₉₃₄—O—Si(CH₃)₃, (CH₃)₃Si—[O—Si(CH₃)₂]₉₃₅—O—Si(CH₃)₃, (CH₃)₃Si—[O—Si(CH₃)₂]₉₃₆—O—Si(CH₃)₃, (CH₃)₃Si—[O—Si(CH₃)₂]₉₃₇—O—Si(CH₃)₃, (CH₃)₃Si—[O—Si(CH₃)₂]₉₃₈—O—Si(CH₃)₃, (CH₃)₃Si—[O—Si(CH₃)₂]₉₃₉—O—Si(CH₃)₃, (CH₃)₃Si—[O—Si(CH₃)₂]₉₄₀—O—Si(CH₃)₃, (CH₃)₃Si—[O—Si(CH₃)₂]₉₄₁—O—Si(CH₃)₃, (CH₃)₃Si—[O—Si(CH₃)₂]₉₄₂—O—Si(CH₃)₃, (CH₃)₃Si—[O—Si(CH₃)₂]₉₄₃—O—Si(CH₃)₃, (CH₃)₃Si—[O—Si(CH₃)₂]₉₄₄—O—Si(CH₃)₃, (CH₃)₃Si—[O—Si(CH₃)₂]₉₄₅—O—Si(CH₃)₃, (CH₃)₃Si—[O—Si(CH₃)₂]₉₄₆—O—Si(CH₃)₃, (CH₃)₃Si—[O—Si(CH₃)₂]₉₄₇—O—Si(CH₃)₃, (CH₃)₃Si—[O—Si(CH₃)₂]₉₄₈—O—Si(CH₃)₃, (CH₃)₃Si—[O—Si(CH₃)₂]₉₄₉—O—Si(CH₃)₃, (CH₃)₃Si—[O—Si(CH₃)₂]₉₅₀—O—Si(CH₃)₃, (CH₃)₃Si—[O—Si(CH₃)₂]₉₅₁—O—Si(CH₃)₃, (CH₃)₃Si—[O—Si(CH₃)₂]₉₅₂—O—Si(CH₃)₃, (CH₃)₃Si—[O—Si(CH₃)₂]₉₅₃—O—Si(CH₃)₃, (CH₃)₃Si—[O—Si(CH₃)₂]₉₅₄—O—Si(CH₃)₃, (CH₃)₃Si—[O—Si(CH₃)₂]₉₅₅—O—Si(CH₃)₃, (CH₃)₃Si—[O—Si(CH₃)₂]₉₅₆—O—Si(CH₃)₃, (CH₃)₃Si—[O—Si(CH₃)₂]₉₅₇—O—Si(CH₃)₃, (CH₃)₃Si—[O—Si(CH₃)₂]₉₅₈—O—Si(CH₃)₃, (CH₃)₃Si—[O—Si(CH₃)₂]₉₅₉—O—Si(CH₃)₃, (CH₃)₃Si—[O—Si(CH₃)₂]₉₆₀—O—Si(CH₃)₃, (CH₃)₃Si—[O—Si(CH₃)₂]₉₆₁—O—Si(CH₃)₃, (CH₃)₃Si—[O—Si(CH₃)₂]₉₆₂—O—Si(CH₃)₃, (CH₃)₃Si—[O—Si(CH₃)₂]₉₆₃—O—Si(CH₃)₃, (CH₃)₃Si—[O—Si(CH₃)₂]₉₆₄—O—Si(CH₃)₃, (CH₃)₃Si—[O—Si(CH₃)₂]₉₆₅—O—Si(CH₃)₃, (CH₃)₃Si—[O—Si(CH₃)₂]₉₆₆—O—Si(CH₃)₃, (CH₃)₃Si—[O—Si(CH₃)₂]₉₆₇—O—Si(CH₃)₃, (CH₃)₃Si—[O—Si(CH₃)₂]₉₆₈—O—Si(CH₃)₃, (CH₃)₃Si—[O—Si(CH₃)₂]₉₆₉—O—Si(CH₃)₃, (CH₃)₃Si—[O—Si(CH₃)₂]₉₇₀—O—Si(CH₃)₃, (CH₃)₃Si—[O—Si(CH₃)₂]₉₇₁—O—Si(CH₃)₃, (CH₃)₃Si—[O—Si(CH₃)₂]₉₇₂—O—Si(CH₃)₃, (CH₃)₃Si—[O—Si(CH₃)₂]₉₇₃—O—Si(CH₃)₃, (CH₃)₃Si—[O—Si(CH₃)₂]₉₇₄—O—Si(CH₃)₃, (CH₃)₃Si—[O—Si(CH₃)₂]₉₇₅—O—Si(CH₃)₃, (CH₃)₃Si—[O—Si(CH₃)₂]₉₇₆—O—Si(CH₃)₃, (CH₃)₃Si—[O—Si(CH₃)₂]₉₇₇—O—Si(CH₃)₃, (CH₃)₃Si—[O—Si(CH₃)₂]₉₇₈—O—Si(CH₃)₃, (CH₃)₃Si—[O—Si(CH₃)₂]₉₇₉—O—Si(CH₃)₃, (CH₃)₃Si—[O—Si(CH₃)₂]₉₈₀—O—Si(CH₃)₃, (CH₃)₃Si—[O—Si(CH₃)₂]₉₈₁—O—Si(CH₃)₃, (CH₃)₃Si—[O—Si(CH₃)₂]₉₈₂—O—Si(CH₃)₃, (CH₃)₃Si—[O—Si(CH₃)₂]₉₈₃—O—Si(CH₃)₃, (CH₃)₃Si—[O—Si(CH₃)₂]₉₈₄—O—Si(CH₃)₃, (CH₃)₃Si—[O—Si(CH₃)₂]₉₈₅—O—Si(CH₃)₃, (CH₃)₃Si—[O—Si(CH₃)₂]₉₈₆—O—Si(CH₃)₃, (CH₃)₃Si—[O—Si(CH₃)₂]₉₈₇—O—Si(CH₃)₃, (CH₃)₃Si—[O—Si(CH₃)₂]₉₈₈—O—Si(CH₃)₃, (CH₃)₃Si—[O—Si(CH₃)₂]₉₈₉—O—Si(CH₃)₃, (CH₃)₃Si—[O—Si(CH₃)₂]₉₉₀—O—Si(CH₃)₃, (CH₃)₃Si—[O—Si(CH₃)₂]₉₉₁—O—Si(CH₃)₃, (CH₃)₃Si—[O—Si(CH₃)₂]₉₉₂—O—Si(CH₃)₃, (CH₃)₃Si—[O—Si(CH₃)₂]₉₉₃—O—Si(CH₃)₃, (CH₃)₃Si—[O—Si(CH₃)₂]₉₉₄—O—Si(CH₃)₃, (CH₃)₃Si—[O—Si(CH₃)₂]₉₉₅—O—Si(CH₃)₃, (CH₃)₃Si—[O—Si(CH₃)₂]₉₉₆—O—Si(CH₃)₃, (CH₃)₃Si—[O—Si(CH₃)₂]₉₉₇—O—Si(CH₃)₃, (CH₃)₃Si—[O—Si(CH₃)₂]₉₉₈—O—Si(CH₃)₃, (CH₃)₃Si—[O—Si(CH₃)₂]₉₉₉—O—Si(CH₃)₃, (CH₃)₃Si—[O—Si(CH₃)₂]₁₀₀₀—O—Si(CH₃)₃, (CH₃)₃Si—[O—Si(CH₃)₂]₁₀₀₁—O—Si(CH₃)₃, (CH₃)₃Si—[O—Si(CH₃)₂]₁₀₀₂—O—Si(CH₃)₃, (CH₃)₃Si—[O—Si(CH₃)₂]₁₀₀₃—O—Si(CH₃)₃, (CH₃)₃Si—[O—Si(CH₃)₂]₁₀₀₄—O—Si(CH₃)₃, (CH₃)₃Si—[O—Si(CH₃)₂]₁₀₀₅—O—Si(CH₃)₃, (CH₃)₃Si—[O—Si(CH₃)₂]₁₀₀₆—O—Si(CH₃)₃, (CH₃)₃Si—[O—Si(CH₃)₂]₁₀₀₇—O—Si(CH₃)₃, (CH₃)₃Si—[O—Si(CH₃)₂]₁₀₀₈—O—Si(CH₃)₃, (CH₃)₃Si—[O—Si(CH₃)₂]₁₀₀₉—O—Si(CH₃)₃, (CH₃)₃Si—[O—Si(CH₃)₂]₁₀₁₀—O—Si(CH₃)₃, (CH₃)₃Si—[O—Si(CH₃)₂]₁₀₁₁—O—Si(CH₃)₃, (CH₃)₃Si—[O—Si(CH₃)₂]₁₀₁₂—O—Si(CH₃)₃, (CH₃)₃Si—[O—Si(CH₃)₂]₁₀₁₃—O—Si(CH₃)₃, (CH₃)₃Si—[O—Si(CH₃)₂]₁₀₁₄—O—Si(CH₃)₃, (CH₃)₃Si—[O—Si(CH₃)₂]₁₀₁₅—O—Si(CH₃)₃, (CH₃)₃Si—[O—Si(CH₃)₂]₁₀₁₆—O—Si(CH₃)₃, (CH₃)₃Si—[O—Si(CH₃)₂]₁₀₁₇—O—Si(CH₃)₃, (CH₃)₃Si—[O—Si(CH₃)₂]₁₀₁₈—O—Si(CH₃)₃, (CH₃)₃Si—[O—Si(CH₃)₂]₁₀₁₉—O—Si(CH₃)₃, (CH₃)₃Si—[O—Si(CH₃)₂]₁₀₂₀—O—Si(CH₃)₃, (CH₃)₃Si—[O—Si(CH₃)₂]₁₀₂₁—O—Si(CH₃)₃, (CH₃)₃Si—[O—Si(CH₃)₂]₁₀₂₂—O—Si(CH₃)₃, (CH₃)₃Si—[O—Si(CH₃)₂]₁₀₂₃—O—Si(CH₃)₃, (CH₃)₃Si—[O—Si(CH₃)₂]₁₀₂₄—O—Si(CH₃)₃, (CH₃)₃Si—[O—Si(CH₃)₂]₁₀₂₅—O—Si(CH₃)₃, (CH₃)₃Si—[O—Si(CH₃)₂]₁₀₂₆—O—Si(CH₃)₃, (CH₃)₃Si—[O—Si(CH₃)₂]₁₀₂₇—O—Si(CH₃)₃, (CH₃)₃Si—[O—Si(CH₃)₂]₁₀₂₈—O—Si(CH₃)₃, (CH₃)₃Si—[O—Si(CH₃)₂]₁₀₂₉—O—Si(CH₃)₃, (CH₃)₃Si—[O—Si(CH₃)₂]₁₀₃₀—O—Si(CH₃)₃, (CH₃)₃Si—[O—Si(CH₃)₂]₁₀₃₁—O—Si(CH₃)₃, (CH₃)₃Si—[O—Si(CH₃)₂]₁₀₃₂—O—Si(CH₃)₃, (CH₃)₃Si—[O—Si(CH₃)₂]₁₀₃₃—O—Si(CH₃)₃, (CH₃)₃Si—[O—Si(CH₃)₂]₁₀₃₄—O—Si(CH₃)₃, (CH₃)₃Si—[O—Si(CH₃)₂]₁₀₃₅—O—Si(CH₃)₃, (CH₃)₃Si—[O—Si(CH₃)₂]₁₀₃₆—O—Si(CH₃)₃, (CH₃)₃Si—[O—Si(CH₃)₂]₁₀₃₇—O—Si(CH₃)₃, (CH₃)₃Si—[O—Si(CH₃)₂]₁₀₃₈—O—Si(CH₃)₃, (CH₃)₃Si—[O—Si(CH₃)₂]₁₀₃₉—O—Si(CH₃)₃, (CH₃)₃Si—[O—Si(CH₃)₂]₁₀₄₀—O—Si(CH₃)₃, (CH₃)₃Si—[O—Si(CH₃)₂]₁₀₄₁—O—Si(CH₃)₃, (CH₃)₃Si—[O—Si(CH₃)₂]₁₀₄₂—O—Si(CH₃)₃, (CH₃)₃Si—[O—Si(CH₃)₂]₁₀₄₃—O—Si(CH₃)₃, (CH₃)₃Si—[O—Si(CH₃)₂]₁₀₄₄—O—Si(CH₃)₃, (CH₃)₃Si—[O—Si(CH₃)₂]₁₀₄₅—O—Si(CH₃)₃, (CH₃)₃Si—[O—Si(CH₃)₂]₁₀₄₆—O—Si(CH₃)₃, (CH₃)₃Si—[O—Si(CH₃)₂]₁₀₄₇—O—Si(CH₃)₃, (CH₃)₃Si—[O—Si(CH₃)₂]₁₀₄₈—O—Si(CH₃)₃, (CH₃)₃Si—[O—Si(CH₃)₂]₁₀₄₉—O—Si(CH₃)₃, (CH₃)₃Si—[O—Si(CH₃)₂]₁₀₅₀—O—Si(CH₃)₃, (CH₃)₃Si—[O—Si(CH₃)₂]₁₀₅₁—O—Si(CH₃)₃, (CH₃)₃Si—[O—Si(CH₃)₂]₁₀₅₂—O—Si(CH₃)₃, (CH₃)₃Si—[O—Si(CH₃)₂]₁₀₅₃—O—Si(CH₃)₃, (CH₃)₃Si—[O—Si(CH₃)₂]₁₀₅₄—O—Si(CH₃)₃, (CH₃)₃Si—[O—Si(CH₃)₂]₁₀₅₅—O—Si(CH₃)₃, (CH₃)₃Si—[O—Si(CH₃)₂]₁₀₅₆—O—Si(CH₃)₃, (CH₃)₃Si—[O—Si(CH₃)₂]₁₀₅₇—O—Si(CH₃)₃, (CH₃)₃Si—[O—Si(CH₃)₂]₁₀₅₈—O—Si(CH₃)₃, (CH₃)₃Si—[O—Si(CH₃)₂]₁₀₅₉—O—Si(CH₃)₃, (CH₃)₃Si—[O—Si(CH₃)₂]₁₀₆₀—O—Si(CH₃)₃, (CH₃)₃Si—[O—Si(CH₃)₂]₁₀₆₁—O—Si(CH₃)₃, (CH₃)₃Si—[O—Si(CH₃)₂]₁₀₆₂—O—Si(CH₃)₃, (CH₃)₃Si—[O—Si(CH₃)₂]₁₀₆₃—O—Si(CH₃)₃, (CH₃)₃Si—[O—Si(CH₃)₂]₁₀₆₄—O—Si(CH₃)₃, (CH₃)₃Si—[O—Si(CH₃)₂]₁₀₆₅—O—Si(CH₃)₃, (CH₃)₃Si—[O—Si(CH₃)₂]₁₀₆₆—O—Si(CH₃)₃, (CH₃)₃Si—[O—Si(CH₃)₂]₁₀₆₇—O—Si(CH₃)₃, (CH₃)₃Si—[O—Si(CH₃)₂]₁₀₆₈—O—Si(CH₃)₃, (CH₃)₃Si—[O—Si(CH₃)₂]₁₀₆₉—O—Si(CH₃)₃, (CH₃)₃Si—[O—Si(CH₃)₂]₁₀₇₀—O—Si(CH₃)₃, (CH₃)₃Si—[O—Si(CH₃)₂]₁₀₇₁—O—Si(CH₃)₃, (CH₃)₃Si—[O—Si(CH₃)₂]₁₀₇₂—O—Si(CH₃)₃, (CH₃)₃Si—[O—Si(CH₃)₂]₁₀₇₃—O—Si(CH₃)₃, (CH₃)₃Si—[O—Si(CH₃)₂]₁₀₇₄—O—Si(CH₃)₃, (CH₃)₃Si—[O—Si(CH₃)₂]₁₀₇₅—O—Si(CH₃)₃, (CH₃)₃Si—[O—Si(CH₃)₂]₁₀₇₆—O—Si(CH₃)₃, (CH₃)₃Si—[O—Si(CH₃)₂]₁₀₇₇—O—Si(CH₃)₃, (CH₃)₃Si—[O—Si(CH₃)₂]₁₀₇₈—O—Si(CH₃)₃, (CH₃)₃Si—[O—Si(CH₃)₂]₁₀₇₉—O—Si(CH₃)₃, (CH₃)₃Si—[O—Si(CH₃)₂]₁₀₈₀—O—Si(CH₃)₃, (CH₃)₃Si—[O—Si(CH₃)₂]₁₀₈₁—O—Si(CH₃)₃, (CH₃)₃Si—[O—Si(CH₃)₂]₁₀₈₂—O—Si(CH₃)₃, (CH₃)₃Si—[O—Si(CH₃)₂]₁₀₈₃—O—Si(CH₃)₃, (CH₃)₃Si—[O—Si(CH₃)₂]₁₀₈₄—O—Si(CH₃)₃, (CH₃)₃Si—[O—Si(CH₃)₂]₁₀₈₅—O—Si(CH₃)₃, (CH₃)₃Si—[O—Si(CH₃)₂]₁₀₈₆—O—Si(CH₃)₃, (CH₃)₃Si—[O—Si(CH₃)₂]₁₀₈₇—O—Si(CH₃)₃, (CH₃)₃Si—[O—Si(CH₃)₂]₁₀₈₈—O—Si(CH₃)₃, (CH₃)₃Si—[O—Si(CH₃)₂]₁₀₈₉—O—Si(CH₃)₃, (CH₃)₃Si—[O—Si(CH₃)₂]₁₀₉₀—O—Si(CH₃)₃, (CH₃)₃Si—[O—Si(CH₃)₂]₁₀₉₁—O—Si(CH₃)₃, (CH₃)₃Si—[O—Si(CH₃)₂]₁₀₉₂—O—Si(CH₃)₃, (CH₃)₃Si—[O—Si(CH₃)₂]₁₀₉₃—O—Si(CH₃)₃, (CH₃)₃Si—[O—Si(CH₃)₂]₁₀₉₄—O—Si(CH₃)₃, (CH₃)₃Si—[O—Si(CH₃)₂]₁₀₉₅—O—Si(CH₃)₃, (CH₃)₃Si—[O—Si(CH₃)₂]₁₀₉₆—O—Si(CH₃)₃, (CH₃)₃Si—[O—Si(CH₃)₂]₁₀₉₇—O—Si(CH₃)₃, (CH₃)₃Si—[O—Si(CH₃)₂]₁₀₉₈—O—Si(CH₃)₃, (CH₃)₃Si—[O—Si(CH₃)₂]₁₀₉₉—O—Si(CH₃)₃, (CH₃)₃Si—[O—Si(CH₃)₂]₁₁₀₀—O—Si(CH₃)₃, (CH₃)₃Si—[O—Si(CH₃)₂]—O—Si(CH₃)₃

and mixtures of these substances.

In addition to, or in place of the silicones of the formula Si-I, the agents according to the invention can comprise at least one silicone of the formula Si-II

(CH₃)₃Si—[O—Si(CH₃)₂]ₓ—[O—(H₃C)Si((CH₂)ₖ(CH₂CH₂O)ₘ(CH₂CH₂CH₂O)ₙ)]ₓ—O—Si(CH₃)₃ (Si-II),

in which
k stands for values of 1 to 20, preferably 2 to 10, and especially 2, 3, 4, 5, 6;
m stands for values of 0 to 100, preferably 2 to 40, especially preferably 5 to 35 and particularly 8, 9, 10, 11, 12, 13, 14, 15, 16, 17, 18, 19, 20;
n stands for values of 0 to 100, preferably 0 to 50, especially preferably 0 to 35, and particularly for 0, 8, 9, 10, 11, 12, 13, 14, 15, 16, 17, 18, 19 or 20;
x and y, independently of each other, stand for a number from 0 to 5,000, preferably from 10 to 2,500, further preferably from 50 to 1,500 and particularly 100 to 1000,
   provided that (m+n) is not equal to zero.

In these silicones, a methyl group of some dimethylsiloxane repeating units is replaced by an ethoxylated and/or propoxylated alkylene group, so that these silicones are water-soluble. According to the INCI nomenclature, silicones having the formula Si-II are called Dimethicone copolyols.

Hair treatment agents preferred according to the invention are accordingly distinguished by comprising, based on its weight, 0.01 to 5% by weight, preferably 0.025 to 2.5% by weight, especially preferably 0.05 to 1% by weight, further preferably 0.075 to 0.75% by weight, and particularly 0.1 to 0.25% by weight of at least one aminofunctional silicone of the formula (Si-II)

R′ₐG₃₋ₐ-Si(OSiG₂)ₙ-(OSiG_{b}R′_{2-b})ₘ—O—SiG₃₋ₐ-R′ₐ (Si-II),

in which
G is —H, a phenyl group, —OH, —O—CH₃, —CH₃, —CH₂CH₃, —CH₂CH₂—CH₃, —CH(CH₃)₂, —CH(CH₃)CH₂CH₃, or —C(CH₃)₃;
a stands for a number between 0 and 3, particularly 0,
b stands for a number between 0 and 1, particularly 1,
m and n are numbers for which the sum (m+n) is between 1 and 2,000, preferably between 50 and 150, with n preferably having values of 0 to 1999 and particularly from 49 to 149, and m preferably having values of 1 to 2,000, particularly 1 to 10,
R′ is a monovalent group selected from
   —N(R″)—CH₂—CH₂—N(R″)₂
   —N(R″)₂
   —N⁺(R″)₃A⁻
   —N⁺H(R″)₂A⁻
   —N⁺H₂(R″)A⁻
   —N(R″)—CH₂—CH₂—N⁺R″H₂A⁻,
in which each R″ stands for same or different groups from the group —H, -phenyl, -benzyl-, the C₁₋₂₀ alkyl group, preferably —CH₃, —CH₂CH₃, —CH₂CH₂—CH₃, —CH(CH₃)₂, —CH₂CH₂CH₂CH₃, —CH₂—CH(CH₃)₂, —CH(CH₃)CH₂CH₃, or —C(CH₃)₃, and A represents an anion that is preferably selected from chloride, bromide, iodide or methosulfate.

Agents preferred according to the invention comprise Dimethicone polyols, i.e., silicones of the formula Si-II, in narrow quantitative ranges. Here, cosmetic agents according to the invention are preferred which comprise, based on its weight, 0.02 to 8.5% by weight, preferably 0.1 to 7.5% by weight, especially preferably 0.25 to 5% by weight, further preferably 0.25 to 4% by weight, and particularly 0.3 to 2.5% by weight of at least one silicone of formula Si-II.

The silicones of formula Si-II can also have degrees of oligomerization or polymerization between 0 and 5,000, with preferred degrees of oligomerization or polymerization being in the range of 10 to 2,500, further preferably 50 to 1,500, and particularly 100 to 1,000. Of course, the molecular weights of the Dimethicone polyols likewise vary, of course, with the degree of oligomerization or polymerization.

According to the invention, Dimethicone polyols comprising the agents have the polymerization degree x (for the unsubstituted dimethylsiloxane unit) or y (for the substituted dimethylsiloxane unit). Although “total” degrees of polymerization (sum of x+y) of up to 10,000 are theoretically possible, agents preferred according to the invention are those for which the sum x+y is in the range of 10 to 2,500, further preferably from 50 to 1,500 and particularly from 100 to 1,000. The representation in the formula Si-II is schematic. It does not mean that the substituted and unsubstituted dimethylsiloxane units must be in blocks. Rather, the —[—SI(CH₃)₂]ₓ— units and the —[O—(H₃C)Si((CH₂)ₖ(CH₂CH₂O)ₘ(CH₂CH₂CH₂O)ₙ)]_{y} units can even be “randomized”; that is, randomly distributed through the molecule.

The viscosity of the Dimethicone polyol used according to the invention is likewise nearly independent of the temperature and varies with the molecular weight, with high-molecular-weight Dimethicone polyols having higher viscosities than those with lower molecular weight. Here, cosmetic agents according to the invention are preferred that contain, as the silicone of formula Si-II a Dimethicone polyol having a viscosity of 1 to 10,000 cSt, preferably 10 to 5,000 cSt, especially preferably 50 to 2,500 cSt, further preferably 100 to 1,000 cSt and particularly 300 to 500 cSt (measured at 20° C.).

The degree of ethoxylation, m, according to the invention is 0 to 100, preferably 2 to 50, especially preferably 5 to 35 and particularly 8, 9, 10, 11, 12, 13, 14, 15, 16, 17, 18, 19 or 20. The degree of propoxylation, n, stands for values of 0 to 100, preferably 0 to 50, especially preferably 0 to 35, and particularly for 0, 8, 9, 10, 11, 12, 13, 14, 15, 16, 17, 18, 19 or 20. Here, too, the representation of the formula Si-II does not mean that the ethylene oxide units must necessarily be bound to the alkylene group linked to the Si atom. Rather, the EO and PO units are interchangeable, so that a —[O—(H₃C)Si((CH₂)ₖ(CH₂CH₂CH₂O)ₘ(CH₂CH₂O)ₙ)]_{y} unit is also possible. Here, again, the sequence EOₘPOₙ does not necessarily mean that the EO and PO units must occur in blocks. Rather, the EO and PO units can also be “randomized”, that is, be randomly distributed through the molecule.

The alkylene group in formula Si-II is preferably a linear alkylene group in which k stands for values of 1 to 20, preferably 2 to 10, and particularly for 2, 3, 4, 5, 6. Thus the groupings

—CH₂—, —CH₂—CH₂—, —H₂—CH₂—CH₂—, —CH₂—CH₂—CH₂—CH₂—, —CH₂—CH₂—CH₂—CH₂—CH₂—, —CH₂—CH₂—CH₂—CH₂—CH₂—CH₂—CH₂—CH₂—CH₂—CH₂—.
are preferred.

The silicones of formula Si-I and those of formula Si-II are preferably used in certain relative weight ratios. Here, cosmetic agents according to the invention are preferred in which the weight ratio of the silicones of formula Si-I comprising the agent to those of formula Si-II comprising the agent is 10:1 to 1:10, preferably 8:1 to 1:8, especially preferably 7:1 to 1:6, further preferably 4:1 to 1:3 and particularly 3:1 to 1:2.

In addition to, or alternatively to, the silicones of formula (Si-I) and/or (Si—II), the agent according to the invention can also comprise at least one amino-functional silicone, that is, a silicone having at least one (optionally substituted amino group.

Such silicones can be described, for example, by the formula

M(RₐQ_{b}SiO_{(4-a-b)/2)x}(R_{c}SiO_{(4-c)/2)y}M

where R in the formula above is a hydrocarbon or a hydrocarbon group having 1 to about 6 carbon atoms, Q is a polar group having the general formula —R¹HZ, in which R¹ is a bifunctional connecting group bound to hydrogen and the group Z, composed of carbon and hydrogen atoms, carbon, hydrogen and oxygen atoms, or carbon, hydrogen and nitrogen atoms, and Z is an amino-functional organic group containing at least one amino-functional group; “a” takes on values in the range of about 0 to about 2; “b” takes on values in the range of about 1 to about 3; “a”+“b” is less than or equal to 3; and “c” is a number in the range of about 1 to about 3; and x is a number in the range of 1 to about 2,000, preferably from about 3 to about 50, and most preferably from about 3 to about 25; and y is a number in the range of about 20 to about 10,000, preferably from about 125 to about 10,000, and most preferably about 150 to about 1,000; and M is a suitable silicone end-group such as is known at the state of the art, preferably trimethylsiloxy. Non-limiting examples of the groups represented by are include alkyl groups such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl and the like; phenyl groups, benzyl groups, halohydrocarbon groups such as 3-chloropropyl, 4-bromobutyl, 3,3,3-trifluoropropyl, chlorocyclohexyl, bromophenyl, chlorophenyl and the like, as well as sulfur-containing groups such as mercaptoethyl, mercaptopropyl, mercaptohexyl, mercaptophenyl and the like. R is preferably an alkyl group containing 1 to about 6 carbon atoms, and it is most preferable for R to be methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄—C₆H₄—C₆H₄—CH₂—C₆H₄— and —(CH₂)₃C(O)SCH₂CH₂—.

Z is an amino-functional organic group containing at least one functional amino group. One possible formula for Z is NH(CH₂)_{z}NH₂, in which z is one or more. Another possible formula for Z is —NH(CH₂)_{z}(CH₂)_{zz}NH—, in which both z and zz are independently 1 or more, with this structure including diamino ring structures such as piperazinyl. Z is most preferably a —NHCH₂CH₂NH₂ group. Another possible formula for Z is —NH(CH₂)_{z}(CH₂)_{zz}NX₂ or —NX₂, in which each X of the X₂ is independently selected from the group consisting of hydrogen and alkyl groups with 1 to 12 carbon atoms and zz is 0.

Q is most preferably a polar amino-functional group of the formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b}SiO_{(4-a-b)/2} units to the R_{c}SiO_{(4-c)/2} units is in the range of about 1:2 to 1:65, preferably about 1:5 to about 1:65 and most preferably about 1:15 to about 1:20. If one or more silicones of the formula above are used, then the various variable substituents in the formula above can be different for the different silicone components occurring in the silicone mixture.

Preferred agents according to the invention are distinguished by comprising an aminofunctional silicone of formula (I)

R′ₐG₃₋ₐ-Si(OSiG₂)ₙ-(OSiG_{b}R′_{2-b})ₘ—O—SiG₃₋ₐ-R′ₐ (I).

in which
G is —H, a phenyl group, —OH, —O—CH₃, —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, or —C(CH₃)₃;
a stands for a number between 0 and 3, preferably 0;
b stands for a number between 0 and 1, preferably 1;
m and n are numbers having a sum (m+n) between 1 and 2,000, preferably between 50 and 150, in which n preferably has values of 0 to 1,999 and particularly of 49 to 149 and m preferably has values of 1 to 2,000, particularly 1 to 10;
R′ is a monofunctional group selected from
   —N(R″)—CH₂—CH₂—N(R″)₂
   —N(R″)₂
   —N⁺(R″)₃A⁻
   —N⁺H(R″)₂A⁻
   —N⁺H₂(R″)A⁻
   —N(R″)—CH₂—CH₂—N⁺R″H₂A⁻,
   in which each R″ stands for the same or different groups from the group —H, phenyl, benzyl, the C₁₋₂₀-alkyl group, preferably met-alkyl group, preferably —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, or —C(CH₃)₃ and A represents an anion preferably selected from chloride. bromide, iodide or methosulfate.

Hair treatment agents especially preferred according to the invention are distinguished by comprising, based on its weight, 0.01 to 5% by weight, preferably 0.025 to 2.5% by weight, especially preferably 0.05 to 1% by weight, further preferably 0.075 to 0.75% by weight and particularly 0.1 to 0.25% by weight of at least one amino-functional silicone of the formula (Si-III)

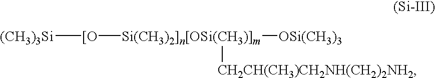

in which m and n are numbers having a sum (m+n) between 1 and 2,000, preferably between 50 and 150, with n preferably having values from 0 to 1,999 and especially from 49 to 149 and m preferably having values of 1 to 2,000, especially from 1 to 10.

These silicones are called trimethylsilylamodimethicones according to the INCI declaration.

Those hair treatment agents according to the invention are also specially preferred that are distinguished by comprising, based on its weight, 0.01 to 5% by weight, preferably 0.025 to 2.5% by weight, especially preferably 0.05 to 1% by weight, further preferably 0.075 to 0.75% by weight and particularly 0.1 to 0.25% by weight of at least one amino-functional silicone of the formula (Si-IV)

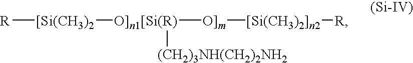

in which R stands for —OH, —O—CH₃ or a —CH₃ group, and m, n1 and n2 are numbers for which the sum (m+n1+n2) is between 1 and 2,000, preferably between 50 and 150, with the sum (n1+n2) preferably taking on values of 0 to 1,999 and particularly from 49 to 149, and m preferably taking on values of 1 to 2,000, particularly from 1 to 10.

These silicones are called Amodimethicones according to the INCI declaration.

Independently of which amino-functional silicones are used, those hair treatment agents are preferred according to the invention for which the amino-functional silicone has an amine value greater than 0.25 meq/g, preferably greater than 0.3 meq/g and particularly greater than 0.4 meq/g. Here the amine value is the milliequivalents of amine per gram of the amino-functional silicone. It can be determined by titration, and is also reported in the units of mg KOH/g.

It has proven to be advantageous to incorporate both the essential components A and B of the agent according to the invention—copolymer(s) and silicone(s)—into the agent according to the invention as a compound. It is particularly preferable to prepare a ready-made emulsion comprising the two essential components A and B. Corresponding hair treatment agents comprising the copolymer A and the silicone B in the form of an emulsion which comprise, based on the weight of the emulsion,
a) 1 to 20% by weight, preferably 2.5 to 15% by weight and particularly 5 to 10% by weight copolymer(s) A
b) 10 to 70% by weight, preferably 20 to 60% by weight and particularly 25 to 50% by weight silicone(s) B
   are preferred according to the invention.

The emulsion may also contain emulsifiers. That is preferred. Preferred emulsifiers are from the groups of nonionic and/or anionic surfactants, with anionic surfactants having proven particularly good in the emulsions to be used preferably. Here preferred hair treatment agents according to the invention are preferred in which the emulsion comprises 0.1 to 10% by weight, preferably 0.25 to 7.5% by weight, and particularly 1 to 5% by weight of preferably anionic surfactants, especially preferably fatty alcohol ether sulfates of the formula

H₃C—(CH₂)ₙ—(OCH₂CH₂)ₖ—OSO₃⁻M⁺

in which n stands for values of 5 to 21, preferably from 7 to 19, especially preferably 9 to 17, and particularly 11 to 13, k stands for values of 1, 2, 3, 4, 5, 6, 7, 8, 9 or 10, preferably for 6, 7 or 8, and particularly for 7, and M represents a cation of the group Na⁺, K⁺, NH₄⁺, ½ Mg²⁺, or ½ Zn²⁺, preferably Na⁺.

A fatty alcohol ether sulfate of the formula

H₃C—(CH₂)₁₁—(OCH₂CH₂)₇—OSO₃⁻Na⁺

(INCI designation: Laureth-7) is used with particular preference in the emulsion. A particularly preferred emulsion for use according to the invention comprises, based on the weight of the emulsion,
0.1 to 10% by weight, preferably 1 to 5% by weight Laureth-7,
10 to 60% by weight, preferably 25 to 50 by weight silicone, preferably a Dimethicone,
1 to 20% by weight, preferably 5 to 10% by weight of a copolymer of the formula (Ic)
with the remainder water or an aqueous solution of other additives.

Especially preferred hair treatment agents according to the invention comprise the emulsion in proportions of 0.05 to 10% by weight, preferably 0.1 to 5% by weight and particularly 0.2 to 1% by weight, based in each case on the hair treatment agent.

As the third essential ingredient, the agents according to the invention comprise a selected care substance. Those are described in the following.

The agents according to the invention can comprise, as the care substance, a substance from the group of monomers, oligomers, and polymers of amino acids, N—(C₂-C₂₄)-acylamino acids, esters and/or the physiologically acceptable salts of those substances.

Amino acids that can preferably be used according to the invention are derived from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cyss), L-carnitine, L-citrulline, L-theanine, 3′,4′-dihydroxy-L-phenylalanine (L-DOPA), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, and L-ornithine. Both the individual amino acids and mixtures can be used.

Agents preferred according to the invention comprise on or more amino acids within narrow quantitative ranges. Here hair treatment agents according to the invention are distinguished by comprising, as care substance C, based on its weight, 0.01 to 5% by weight, preferably 0.02 to 2.5% by weight, especially preferably 0.05 to 1.5% by weight, further preferably 0.075 to 1% by weight and particularly 0.1 to 0.25% by weight amino acid(s), preferably from the group of glycine and/or alanine and/or valine and/or lysine and/or lysine and/or leucine and/or threonine.

Instead of, or in addition to, the care substances named above, the agents according to the invention can also contain at least one care substance from the group of vitamins, provitamins and vitamin precursors of the groups A, B, C, E, H and K and esters of the substances mentioned above.

The group of substances designated as Vitamin A includes retinol (Vitamin A₁) as well as 3,4-didehydroretinol (Vitamin A₂). β-carotene is the provitamin of retinol. Substances that can be considered as Vitamin A components include, for example, Vitamin A acid and its esters, Vitamin A aldehyde and Vitamin A alcohol, as well as its esters, such as the palmitate and the acetate. The agents according to the invention preferably comprise the Vitamin A components in proportions of 0.05-1%, based on the complete preparation.

The Vitamin B group, or the Vitamin B complex, includes, among others
Vitamin B, (thiamine)
Vitamin B₂ (riboflavin)
Vitamin B₃. The compounds nicotinic acid and nicotinic acid amide (niacinamide) are often given this designation. The nicotinic acid amide is preferred according to the invention. The agents used according to the invention preferably contain it in proportions of 0.05 to 1% by weight, based on the complete agent.
Vitamin B₅ (pantothenic acid, panthenol and pantolactone). Of this group, panthenol and/or pantolactone are used preferably. Panthenol derivatives that can be used according to the invention are, in particular, the esters and ethers of panthenol, as well as cationically derivatized panthenols. Examples of individual members of the group include panthenol triacetate, panthenol monoethyl ether and its monoacetate, and the cationic panthenol derivatives disclosed in WO 92/13829. The vitamins of the B₅ type named are preferably used in the agents according to the invention at proportions of 0.05 to 10% by weight, based on the complete agent. Proportions of 0.1-5% by weight are especially preferred.
Vitamin B₆ (pyridoxine, pyridoxamine and pyridoxal).

Vitamin C (ascorbic acid). Vitamin C is used in the agents according to the invention preferably in proportions of 0.1 to 3% by weight, based on the complete agent. Its use as the palmitic acid ester, the glucoside or the phosphate can be preferred. Use in combination with tocopherols can likewise be preferred.

Vitamin E (tocopherols, especially α-tocopherol). Tocopherol and its derivatives, which include in particular the esters such as the acetate, nicotinate, phosphate and succinate, are used in the agents according to the invention, preferably in proportions of 0.05 to 1% by weight, based on the complete agent.

Vitamin F. The concept of “Vitamin F” is usually understood to be the essential fatty acids, especially linoleic acid, linolenic acid and arachidonic acid.

Vitamin H. The compound (3aS,4S,6aR)-2-oxohexahydrothienol-[3,4-d]-imidazole-4-valeric acid is called Vitamin H; but the trivial name ‘biotin’ has become generally accepted. The agents according to the invention preferably comprise biotin at proportions of 0.0001 to 1.0% by weight, especially proportions of 0.001 to 0.01% by weight.

Summarizing, those hair treatment agents according to the invention are preferred that comprise, as care substance C, based on its weight, 0.1 to 5% by weight, preferably 0.2 to 4% by weight, especially preferably 0.25 to 3.5% by weight, further preferably 0.5 to 3% by weight, and particularly 0.5 to 2.5% by weight Vitamins and/or provitamins and/or vitamin precursors, preferably classified in the groups A, B, C, D, E, F and H, with the preferred agent comprising panthenol ((±)-2,4-dihydroxy-N-(3-hydroxypropyl)-3,3-dimethylbutyramide, Provitamin B₅) and/or pantothenic acid (Vitamin B₃, Vitamin B₅), and/or niacin, niacinamide or nicotinamide (Vitamin B₃) and/or L-ascorbic acid (Vitamin C) and/or thiamin (Vitamin B₁) and/or riboflavin (Vitamin B₂, Vitamin G) and/or Biotin (Vitamin B₇, Vitamin H) and/or folic acid (Vitamin B₉, Vitamin Bc or Vitamin M) and/or Vitamin B₆ and/or Vitamin B₁₂.

Instead of, or in addition to, the care substances named above, the agent according to the invention can also comprise a care substance from the group of α-hydroxycarboxylic acids, α-ketocarboxylic acids, β-hydroxycarboxylic acids and their esters, lactones or salt forms.

Instead of, or in addition to, the care substances named above, the agent according to the invention can also comprise at least one care substance from the group of ubiquinone and ubiquinol and their derivatives.

Agents are specially preferred that comprise, based on their weight, 0.0001 to 5% of at least one bioquinone of the formula (Ch-I)

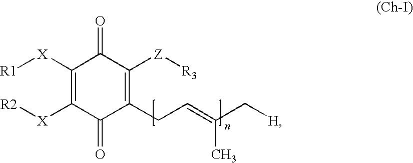

in which
X, Y, Z independently of each other stand for —O— or —NH— or NR⁴— or a chemical bond
R¹, R², R³ independently of each other, stand for a hydrogen atom or for an optionally substituted aryl group or an optionally substituted (C₁-C₆)-alkyl group or a hydroxyalkyl group or a polyhydroxyalkyl group or an optionally substituted (C₁-C₆)-alkylene group or a (C₁-C₆) acyl group, with preferred groups selected, independently of each other, from —H, —CH₃, —CH₂CH₃, —(CH₂)₂CH₂, —CH(CH₃)₂, —(CH₂)₃CH₃, —CH(CH₃)CH₂CH₃, —CH₂—CH(CH₃)₂, or —C(CH₃)₃
R⁴ stands for —CH₃, —CH₂CH₃, —(CH₂)₂CH₂, —CH(CH₃)₂, —(CH₂)₃CH₃, —CH(CH₃)CH₂CH₃, —CH₂—CH(CH₃)₂, or —C(CH₃)₃
n stands for values of 1 to 20, preferably 2 to 15 and particularly for 5, 6, 7, 8, 9, or 10.

Examples of preferred compounds of formula (Ch-I) according to the invention include

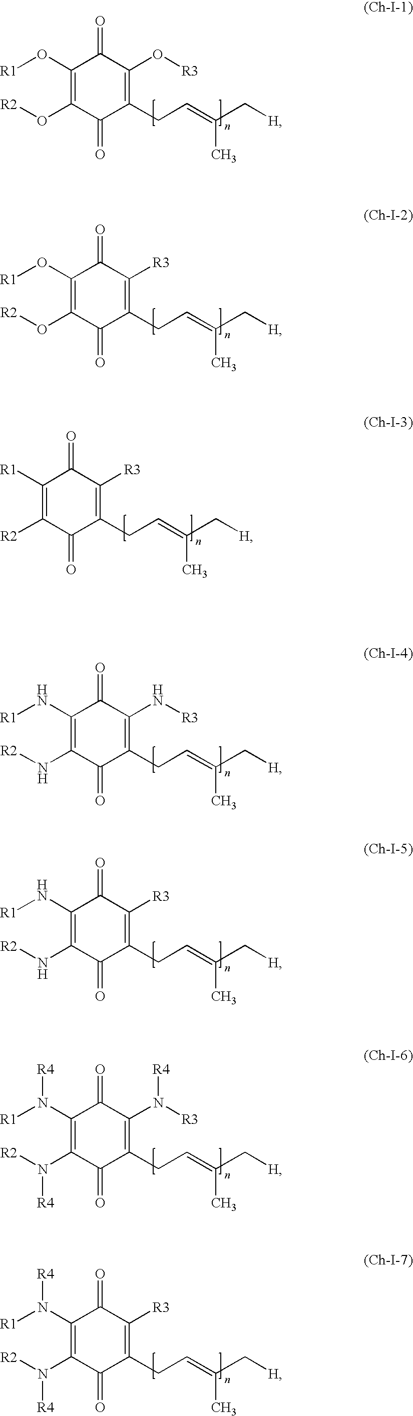

in which
R¹, R², R³ independently of each other, stand for —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃
R⁴ stands for —CH₃ or —CH₂CH₃ or —(CH₂)₂CH₂ or —CH(CH₃)₂
n stands for values of 1 to 20, preferably 2 to 15 and particularly for 5, 6, 7, 8, 9, 10.

Particularly preferred agents according to the invention are distinguished by comprising 0.0002 to 4% by weight, preferably 0.0005 to 3% by weight, especially preferably 0.001 to 2% by weight, further preferably 0.0015 to 1 and particularly 0.002 to 0.5% by weight of at least one ubiquinone of formula (Ch-Ia)

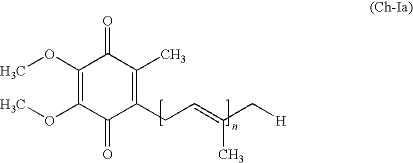

in which n stands for values of 1 to 20, preferably 2 to 15 and particularly for 5, 6, 7, 8, 9, or 10, with particularly preferred agents comprising Coenzyme Q10 of the formula

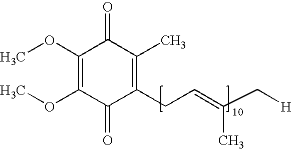

In addition to, or alternately to, the specially preferred, the agents according to the invention can also comprise plastoquinone. Here preferred agents according to the invention are distinguished by comprising 0.0002 to 4% by weight, preferably 0.0005 to 3% by weight, especially preferably 0.001 to 2% by weight, further preferably 0.0015 to 1 and particularly 0.002 to 0.5% by weight of at least one plastoquinone of formula (Ch-1b)

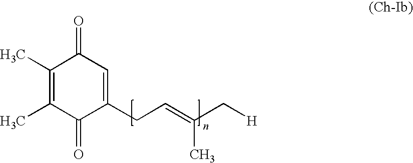

in which n stands for values of 1 to 20, preferably 2 to 15 and particularly for 5, 6, 7, 8, 9, or 10, with particularly preferred agents comprising plastoquinone PQ-9 of the formula

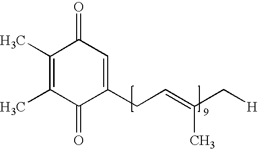

Summarizing, those hair treatment agents according to the invention are preferred that comprise as care substance C, based on its weight, 0.0001 to 1% by weight, preferably 0.001 to 0.5% by weight, and especially preferably 0.005 to 0.1% by weight of at least one ubiquinone and/or at least one ubiquinol and/or at least one derivative of these substances, with preferred agents comprising a ubiquinone of the formula (Ubi)

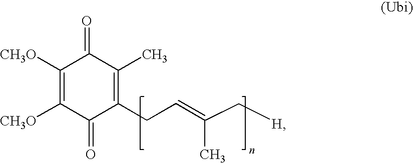

in which n stands for the values 6, 7, 8, 9 or 10, especially preferably for 10 (Coenzyme Q10).

Instead of, or in addition to, the care substances named above, the agents according to the invention can also comprise at least one care substance from the group of purine and/or purine derivatives.

Purine (7H-imidazol-[4,5-d]pyrimidine) does not occur free in nature, but it is the basis of the purines. Purines, in turn, are a group of important compounds that are widely distributed in nature and that participate in human, animal, plant and microbial metabolic processes. They are derived from the basic structure by substitution with OH, NH₂, or SH in the 2-, 6- and 8-positions and/or with CH₃ in the 1-, 3-, and 7-positions. Purine can, for instance, be synthesized from aminoacetonitrile and formamide. Purine and purine derivatives are often isolated from natural substances, but are also accessible synthetically by many routes.

Preferred agents according to the invention comprise purine and/or purine derivatives in narrow weight ranges. Here agents preferred according to the invention are distinguished in comprising, based on its weight, 0.001 to 2.5% by weight, preferably 0.0025 to 1% by weight, especially preferably 0.005 to 0.5% by weight, and particularly 0.01 to 0.1% by weight purine and/or purine derivatives.

Among purine and purine derivatives, some members are specially preferred according to the invention. Agents preferred according to the invention are distinguished in comprising purines and/or purine derivatives having the formula (Pur-1)

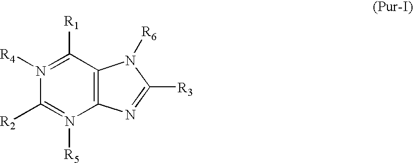

in which the groups R¹, R², and R³, independently of each other, are selected from —H, —OH, —NH₂, and —SH, and the groups R⁴, R⁵ and R⁶ are selected, independently of each other from —H, —CH₃, and —CH₂CH₃, with the following compounds preferred:

purine (R¹═R²═R³═R⁴═R⁵═R⁶═H)

adenine (R¹═NH₂, R²═R³═R⁴═R⁵═R⁶═H)

guanine (R¹═NH₂, R²═R³═R⁴═R⁵═R⁶═H)

uric acid (R¹═R²═R³═OH, R⁴═R⁵═R⁶═H)

hypoxanthine (R¹═OH, R²═R³═R⁴═R⁵═R⁶═H)

purinethiol (R¹═SH, R²═NH₂, R³═R⁴═R⁵═R⁶═H)

6-thioguanine (R¹═SH, R²═NH₂, R³═R⁴═R⁵═R⁶═H)

xanthine (R¹═R²═OH, R³═R⁴═R⁵═R⁶═H)

caffeine (R¹═R²═OH, R³H, R⁴═R⁵═R⁶═CH₃)

theobromine (R¹═R²═OH, R³═R⁴═H, R⁵═R⁶═H)

theophylline (R¹═R²═OH, R³═H, R⁴═CH₃, R⁵═CH₃, R⁶═H)

The nature and amount of the purine derivative can vary, depending on the intended application of the cosmetic agent. Caffeine, in particular, has proven to be good in hair cosmetic formulations. It can be used in shampoos, for example, preferably in proportions of 0.005 to 0.25% by weight, further preferably 0.01 to 0.1% by weight and particularly 0.01 to 0.05% by weight (based on the shampoo in each case).

Summarizing, hair treatment agents preferred according to the invention are distinguished by comprising as care substance C, based on its weight, 0.001 to 2.5% by weight, preferably 0.0025 to 1% by weight, especially preferably 0.005 to 0.5% by weight, and particularly 0.01 to 0.1% by weight purines and/or purine derivatives, preferred agents comprise purine and/or purine derivatives of the formula (Pur-I)

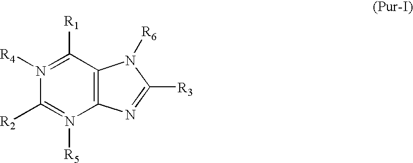

in which the groups R¹, R², and R³, independently of each other, are selected from —H, —OH, NH₂, —SH and the groups R⁴, R⁵, and R⁶, independently of each other, are selected from —H, —CH₃ and —CH₂CH₃, with the following compounds preferred:

purine (R¹═R²═R³═R⁴═R⁵═R⁶═H)

adenine (R¹═NH₂, R²═R³═R⁴═R⁵═R⁶═H)

guanine (R¹═NH₂, R²═R³═R⁴═R⁵═R⁶═H)

uric acid (R¹═R²═R³═OH, R⁴═R⁵═R⁶═H)

hypoxanthine (R¹═OH, R²═R³═R⁴═R⁵═R⁶═H)

6-purinethiol (R¹═SH, R²═R³═R⁴═R⁵═R⁶═H)

6-thioguanine (R¹═SH, R²═NH₂, R³═R⁴═R⁵═R⁶═H)

xanthine (R¹═R²═OH, R³═R⁴═R⁵═R⁶═H)

caffeine (R¹═R²═OH, R³═H, R⁴═R⁵═R⁶═CH₃)

theobromine (R¹═R²═OH, R³═R⁴═H, R⁵═R⁶═H)

theophylline (R¹═R²═OH, R³═H, R⁴═CH₃, R⁵═CH₃, R⁶═H)

Instead of, or in addition to the care substances named above, the agents according to the invention can also comprise at least one care substance from: the group of carbohydrates, selected from monosaccharides, disaccharides and/or oligosaccharides.

Here, hair treatment agents are preferred according to the invention that comprise, as care substance C, and based on its weight, 0.01 to 5% by weight, preferably 0.05 to 4.5% by weight, especially preferably 0.1 to 4% by weight, further preferably 0.5 to 3.5% by weight and particularly 0.75 to 2.5% by weight carbohydrate(s), with preferred carbohydrates selected from

monosaccharides, particularly

D-ribose and/or

D-xylose and/or

L-arabinose and/or

D-glucose and/or

D-mannose and/or

D-galactose and/or

D-fructose and/or

sorbose and/or

L-fucose and/or

L-rhamnose

disaccharides, particularly

sucrose and/or

maltose and/or

lactose and/or

trehalose and/or

cellobiose and/or

gentiobiose and/or

isomaltose.

Instead of, or in addition to, the care substances named above, the agents according to the invention can also contain at least one care substance from the group of hydantoin and/or hydantoin derivatives.

It is especially preferable according to the invention to use hydantoin derivatives, with 5-ureidohydantoin particularly preferred. Independently of whether hydantoin or hydantoin derivative(s) are used, it is preferred to use proportions of 0.02 to 2.5% by weight, preferably 0.05 to 1.5% by weight, further preferably 0.075 to 1% by weight, and particularly 0.1 to 0.25% by weight, based in each case on the complete agent.

Summarizing, those agents are preferred according to the invention that comprise 0.02 to 2.5% by weight, preferably 0.05 to 1.5% by weight, further preferably 0.075 to 1% by weight, and particularly 0.1 to 0.25% by weight of hydantoin and/or hydantoin derivative(s), preferably 5-ureidohydantoin (allantoin)

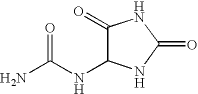

Instead of, or in addition to, the care substances named above, the agents according to the invention can also contain at least one care substance from the group of 2-furanone and/or 2-furanone derivatives.

Here, hair treatment agents are preferred according to the invention that comprise, as care substance C, based on its weight, 0.01 to 15% by weight, preferably 0.025 to 12.5% by weight, especially preferably 0.05 to 10% by weight, further preferably 0.1 to 7.5% by weight and particularly 0.5 to 5% by weight of at least one 2-furanone derivative of formula (Fur-I) and/or formula (Fur-II).

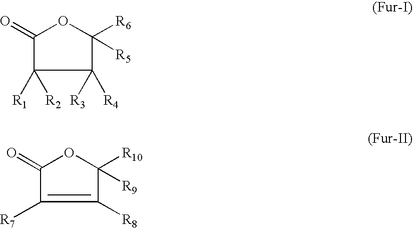

in which the groups R¹ to R¹⁰, independently of each other, stand for:
hydrogen, —OH, a methyl, methoxy, aminomethyl or hydroxymethyl group
a (C₂-C₄) singly or doubly unsaturated, linear or branched hydrocarbon group
a (C₂-C₄) saturated or singly or doubly unsaturated, linear or branched mono, di or trihydroxyhydrocarbon group
a (C₂-C₄) saturated or singly or doubly unsaturated, linear or branched mono, di or triaminohydrocarbon group
a group OR¹¹, in which R¹¹ is a (C₂-C₄) singly or doubly unsaturated, linear or branched hydrocarbon group, a (C₂-C₄) saturated or singly or doubly unsaturated, linear or branched mono, di or trihydroxyhydrocarbon group
a group NR¹²R¹³ in which R¹² and R¹³ each, independently of each other, stand for hydrogen, a methyl, a (C₂-C₄) singly or doubly unsaturated, linear or branched hydrocarbon group or a (C₂-C₄) saturated or singly or doubly unsaturated, linear or branched mono, di or trihydroxyhydrocarbon group
a group —COOR¹⁴, in which R¹⁴ stands for hydrogen, a methyl, a (C₂-C₄) singly or doubly unsaturated, linear or branched hydrocarbon group or a (C₂-C₄) saturated or singly or doubly unsaturated, linear or branched mono, di or trihydroxyhydrocarbon group, or a (C₂-C₄) saturated or singly or doubly unsaturated, linear or branched mono, di or triaminohydrocarbon group
a group —CONR¹⁵R¹⁶ in which R¹⁵ and R¹⁶ each stand for hydrogen, a methyl, a (C₂-C₄) singly or doubly unsaturated, linear or branched hydrocarbon group or a (C₂-C₄) saturated or singly or doubly unsaturated, linear or branched mono, di or trihydroxyhydrocarbon group, or a (C₂-C₄) saturated or singly or doubly unsaturated, linear or branched mono, di or triaminohydrocarbon group
a group —COR¹⁶ in which R¹⁶ stands for a methyl, a (C₂-C₄) singly or doubly unsaturated, linear or branched hydrocarbon group or a (C₂-C₄) saturated or singly or doubly unsaturated, linear or branched mono, di or trihydroxyhydrocarbon group, or a (C₂-C₄) saturated or singly or doubly unsaturated, linear or branched mono, di or triaminohydrocarbon group
a group —OCOR¹⁷ in which R¹⁷ stands for a methyl, a (C₂-C₃₀) singly or doubly unsaturated, linear or branched hydrocarbon group or a (C₂-C₃₀) saturated or singly or doubly unsaturated, linear or branched mono, di, tri or poly-hydroxyhydrocarbon group, or a (C₂-C₃₀) saturated or singly or doubly unsaturated, linear or branched mono, di, tri or poly-aminohydrocarbon group,
   provided that in case R⁷ and R⁸ simultaneously stand for —OH and R⁹ or R¹⁰ stands for hydrogen, the remaining group R⁹ or R¹⁰ does not stand for a dihydroxyethyl group.

2-furanones are known compounds. Their preparation and properties are described, for example, in “Römpp's Lexikon der Chemie, Interactive CD-Rom Version 2.0, under the keyword “dihydro-3-hydroxy-4,4-dimethyl-2(3H)-furanone”, as well as in “Ullmann's Encylopedia, Sixth Edition, 1999, Electronic release”, sections 2.4, 2.7, 3.2, 3.4, 4.3, 6, 11 and 15 and in the literature cited there. Reference is expressly made to those chapters and to the literature cited there. The compounds of formulas (I) and (II) are used as intermediates in natural products synthesis and in production of medications and vitamins. The active substance according to formulas (I) and (II) can be prepared, for instance, by reaction of primary alcohols with acrylic acids. Compounds of formula (I) are also produced by reactions starting with hydroxypivaldehyde. Carbonylations of alkynes also lead to substituted 2-furanones of formulas (I) or (II). Finally, compounds of formula (I) or (II) can be obtained by intramolecular esterification of the corresponding hydroxycarboxylic acids. For example, the following compounds are obtained by one of the synthetic paths indicated above: 2,5-dihydroxy-5-methoxy-2-furanone, tetrahydro-5-oxo-2-furanecarboxylic acid, dihydro-3-hydroxy-4,4-dimethyl-2(3H)-furanone, or 3,4-dimethyl-5-pentylidenedihydro-2(5H)-furanone or 4-hydroxy-2,5-dimethyl-2(2H)-furanone. The 2-furanones according to the invention include, of course, all the possible stereoisomers as well as their mixtures. The 2-furanones according to the invention do not have such a negative effect on the odor of the cosmetic agent as to require perfuming of the agent.

Preferred compounds of the formula (Fur-I) and/or (Fur-2) can be compounds in which the substituents R¹, R², and R⁷, independently of each other, stand for:
hydrogen, —OH, a methyl, methoxy, aminomethyl or hydroxymethyl group
a (C₂-C₄) saturated or singly or doubly unsaturated, linear or branched mono, di or trihydroxyhydrocarbon group
a (C₂-C₄) saturated or singly or doubly unsaturated, linear or branched mono, di or triaminohydrocarbon group
a group OR¹¹, in which R¹¹ is a (C₂-C₄) singly or doubly unsaturated, linear or branched hydrocarbon group, a (C₂-C₄) saturated or singly or doubly unsaturated, linear or branched mono, di or trihydroxyhydrocarbon group
a group NR¹²R¹³ in which R¹² and R¹³ each, independently of each other, stand for hydrogen, a methyl, a (C₂-C₄) singly or doubly unsaturated, linear or branched hydrocarbon group or a (C₂-C₄) saturated or singly or doubly unsaturated, linear or branched mono, di or trihydroxyhydrocarbon group
a group —COOR¹⁴, in which R¹⁴ stands for hydrogen, a methyl, a (C₂-C₄) singly or doubly unsaturated, linear or branched hydrocarbon group or a (C₂-C₄) saturated or singly or doubly unsaturated, linear or branched mono, di or trihydroxyhydrocarbon group, or a (C₂-C₄) saturated or singly or doubly unsaturated, linear or branched mono, di or triaminohydrocarbon group
a group —COR¹⁶ in which R¹⁶ stands for a methyl, a (C₂-C₄) singly or doubly unsaturated, linear or branched hydrocarbon group or a (C₂-C₄) saturated or singly or doubly unsaturated, linear or branched mono, di or trihydroxyhydrocarbon group, or a (C₂-C₄) saturated or singly or doubly unsaturated, linear or branched mono, di or triaminohydrocarbon group
a group —OCOR¹⁷ in which R¹⁷ stands for a methyl, a (C₂-C₃₀) singly or doubly unsaturated, linear or branched hydrocarbon group or a (C₂-C₃₀) saturated or singly or doubly unsaturated, linear or branched mono, di, tri or poly-hydroxyhydrocarbon group, or a (C₂-C₃₀) saturated or singly or doubly unsaturated, linear or branched mono, di, tri or poly-aminohydrocarbon group.

In a further embodiment of the teaching of the invention, it is found that in the compounds of formula (Fur-I) or (Fur-II) it is preferable for the groups R³, R⁴ and R⁸, independently of each other, to stand for:
hydrogen, —OH, a methyl, methoxy, aminomethyl or hydroxymethyl group
a (C₂-C₄) singly or doubly unsaturated, linear or branched hydrocarbon group
a (C₂-C₄) saturated or singly or doubly unsaturated, linear or branched mono, di or trihydroxyhydrocarbon group
a (C₂-C₄) saturated or singly or doubly unsaturated, linear or branched mono, di or triaminohydrocarbon group

It can further be preferred for the groups R⁵, R⁶, R⁹ and R¹⁰ in the active ingredient of formula (I) and/or formula (II) according to the invention to stand for:
hydrogen, an —OH, a methyl, methoxy, aminomethyl or hydroxymethyl group
a (C₂-C₄) singly or doubly unsaturated, linear or branched hydrocarbon group
a (C₂-C₄) saturated or singly or doubly unsaturated, linear or branched mono, di or trihydroxyhydrocarbon group
a (C₂-C₄) saturated or singly or doubly unsaturated, linear or branched mono, di or triaminohydrocarbon group.

A compound of the formula (Fur-I) is used in a specially preferred embodiment of the teaching according to the invention. In this case it can be preferred for the groups R1 and R2 in a compound of formula (I) to stand independently for:
hydrogen, an —OH, a methyl, methoxy, aminomethyl or hydroxymethyl group
a (C₂-C₄) saturated or singly or doubly unsaturated, linear or branched mono, di or trihydroxyhydrocarbon group
a group OR¹¹, in which R¹¹ is a (C₂-C₄) singly or doubly unsaturated, linear or branched hydrocarbon group, or a (C₂-C₄) saturated or singly or doubly unsaturated, linear or branched mono, di or trihydroxyhydrocarbon group
a group —COOR¹⁴, in which R¹⁴ stands for hydrogen, a methyl, a (C₂-C₄) singly or doubly unsaturated, linear or branched hydrocarbon group or a (C₂-C₄) saturated or singly or doubly unsaturated, linear or branched mono, di or trihydroxyhydrocarbon group,
a group —COR¹⁶, in which R¹⁶ stands for a methyl, a (C₂-C₄) saturated or singly or doubly unsaturated, linear or branched hydrocarbon group, a (C₂-C₄) saturated or singly or doubly unsaturated, linear or branched mono, di, or tri-hydroxyhydrocarbon group,
a group —OCOR¹⁷ in which R¹⁷ stands for a methyl, a (C₂-C₃₀) singly or doubly unsaturated, linear or branched hydrocarbon group or a (C₂-C₃₀) saturated or singly or doubly unsaturated, linear or branched mono, di, tri or poly-hydroxyhydrocarbon group,

In this specially preferred embodiment of the teaching according to the invention it can be further preferable for the groups R³ and R⁴ in the compounds according to formula (Fur-1), independently of each other, to stand for:
hydrogen, an —OH, a methyl, methoxy, aminomethyl or hydroxymethyl group
a (C₂-C₄) saturated or singly or doubly unsaturated, linear or branched mono, di or trihydroxyhydrocarbon group
a group OR¹¹, in which R¹¹ is a (C₂-C₄) singly or doubly unsaturated, linear or branched hydrocarbon group, or a (C₂-C₄) saturated or singly or doubly unsaturated, linear or branched mono, di or trihydroxyhydrocarbon group
a group —COOR¹⁴, in which R¹⁴ stands for hydrogen, a methyl, a (C₂-C₄) singly or doubly unsaturated, linear or branched hydrocarbon group or a (C₂-C₄) saturated or singly or doubly unsaturated, linear or branched mono, di or trihydroxyhydrocarbon group,
a group —OCOR¹⁷ in which R¹⁷ stands for a methyl, a (C₂-C₃₀) singly or doubly unsaturated, linear or branched hydrocarbon group or a (C₂-C₃₀) saturated or singly or doubly unsaturated, linear or branched mono, di, tri or poly-hydroxyhydrocarbon group,

In this preferred embodiment it can further be advantageous for the groups R5 and R6 in the compounds according to formula (Fur-I) to stand, independently of each other, for:

hydrogen, an —OH, a methyl, methoxy, aminomethyl or hydroxymethyl group

a (C₂-C₄) saturated or singly or doubly unsaturated, linear or branched mono, di or trihydroxyhydrocarbon group

a group OR¹¹, in which R¹¹ is a (C₂-C₄) singly or doubly unsaturated, linear or branched hydrocarbon group, or a (C₂-C₄) saturated or singly or doubly unsaturated, linear or branched mono, di or trihydroxy-hydrocarbon group

In a specially preferred embodiment of the teaching of the invention,
(R)-(−)-4-hydroxymethyl-γ-buttyrolactone and/or
D,L-4-hydroxymethyl-γ-butyrolactone and/or
(S)-(+)-4-hydroxymethyl-γ-butyrolactone and/or
R-(−)-2-hydroxy-3,3-dimethyl-γ-butyrolactone and/or
D, L-2-hydroxy-3,3-dimethyl-γ-butyrolactone and/or
S-(+)-2-hydroxy-3,3-dimethyl-γ-butyrolactone and/or
4-hydroxy-2,5-dimethyl-3(2H)-furanone and/or
tetrahydro-5-oxo-2-furanecarboxylic acid and/or
tetrahydro-5-oxo-2-furanecarboxylic acid, sodium salt, and/or
tetrahydro-5-oxo-2-furanecarboxylic acid, potassium salt, and/or
2,5-dihydro-5-methoxy-3-furanone and/or
dihydro-3-hydroxy-4,4-dimethyl-2(3H)-furanone
   is used as the compound corresponding to formula (Fur-I). In a very specially preferred embodiment of the teaching according to the invention, dihydro-3-hydroxy-4,4-dimethyl-2(3H)-furanone is used as the compound corresponding to the formula (Fur-I).

In further preferred hair treatment agents according to the invention, 0.01 to 15% by weight, preferably 0.025 to 12.5% by weight, especially preferably 0.05 to 10% by weight, further preferably 0.1 to 7.5% by weight and particularly 0.5 to 5% by weight taurine (2-aminoethanesulfoic acid) is used as care substance C, on the basis of the weight of the hair treatment agent.

Independently of the choice of the copolymer (or copolymers) A, the choice of the silicone (or silicones) B and the care substance (or care substances) C, those hair treatment agents according to the invention are preferred that comprise ingredient A (copolymer) and B (silicone) in the weight ratio A:B of 1:10 to 10:1, preferably 1.8 to 7:1, especially preferably 1:7 to 2:1 and particularly 1:5 to 1:2.

Likewise, those hair treatment agents that comprise the ingredients A (copolymer) and C (care substance) in the weight ratio A:C of 1:1000 to 1000:1, preferably 1:500 to 100:1, especially preferably 1:250 to 75:1, further preferably 1:100 to 50:1 and particularly 1:20 to 5:1 are preferred.

It is also preferable to use also bisabolol and/or bisabolol oxides in the agents according to the invention. Here, hair treatment agents that comprise an additional 0.001 to 5% by weight, preferably 0.01 to 4% by weight, especially preferably 0.02 to 2.5% by weight and particularly 0.1 to 1.5% by weight bisabolol and/or oxides of bisabolol, preferably (−)-alpha-bisabolol

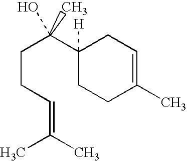

are preferred.

The cosmetic agents according to the invention can further comprise all the active substances, additives, and aids known for such preparations. In many cases the agent comprises at least one surfactant, of which not only anionic but also zwitterionic, ampholytic, nonionic and cationic surfactants are suitable in principle. But in many cases it has proved to be advantageous to select the surfactants from anionic, zwitterionic, or nonionic surfactants. Selected surfactants were described above as components of the emulsion. The description of the other surfactants follows here:

The agents according to the invention preferably comprise at least one emulsifier or surfactant. Surface-active substances are called surfactants or emulsifiers, depending on their application, and are selected from anionic, cationic, zwitterionic, ampholytic and nonionic surfactants and emulsifiers.

All the anionic surface-active substances suitable for use on the human body are suitable as anionic surfactants and emulsifiers for the compositions according to the invention. They are characterized by an anionic group such as a carboxylate, sulfate, sulfonate or phosphate group that makes them water-soluble and a lipophilic alkyl group with about 8 to 30 C atoms. The molecule can also contain glycol or polyglycol ether groups, ester groups, ether groups and amide groups, as well as hydroxyl groups. Examples of suitable anionic surfactants and emulsifiers, each in the form of their sodium, potassium and ammonium salts and of the mono, di, and tri-alkanolammonium salts with 2 to 4 C atoms in the alkanol group are
linear and branched fatty acids with 8 to 30 C atoms (soaps)
ether carboxylic acids of the formula R—O—(CH₂CH₂O)ₓ—CH₂COOH. in which R is a linear alkyl group with 8 to 30 C atoms and x=0 or 1 to 16,
acyl sarcosides with 8 to 24 C atoms in the acyl group
acyl taurides with 8 to 24 C atoms in the acyl group
acyl isethionates with 8 to 24 C atoms in the acyl group
linear alkane sulfonates with 8 to 24 C atoms
linear alpha-olefin sulfonates with 8 to 24 C atoms
alpha-sulfofatty acid methyl esters of fatty acids with 8 to 30 C atoms
acyl glutamates of the formula (T-I):

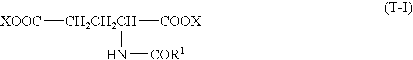

in which the R¹CO stands for a linear or branched acyl group with 6 to 22 C atoms and 0, 1, 2, or 3 double bonds, and X stands for hydrogen, an alkali or alkaline earth metal, ammonium, alkylammonium, alkanolammonium or glucammonium, such as acylglutamates derived from fatty acids having 6 to 22, preferably 12 to 18 carbon atoms, such as C_{12/14}-coco fatty acid or C_{12/18}-coco fatty acid, lauric acid, myristic acid, palmitic acid and/or stearic acid, especially sodium N-cocoyl-L-glutamate and sodium N-stearoyl-L-glutamate esters of a hydroxy-substituted di- or tri-carboxylic acid of the general formula (T-II),

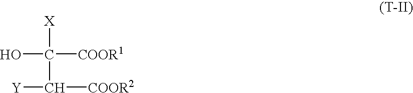

in which X is H or a —CH₂COOR group, Y is H or —OH, provided that Y is H if X is —CH₂COOR, R, R¹ and R², independently of each other, are a hydrogen atom, an alkali or alkaline earth metal cation, an ammonium group, the cation of an ammonium-organic base or a group Z which is selected from a polyhydroxylated organic compound selected from the group of etherified (C₆-C₁₈) alkyl-polysaccharides with 1 to 6 monomeric saccharide units and/or the etherified (C₆-C₁₆) hydroxyalkyl polyols with 2 to 16 hydroxyl groups, provided that at least one of the groups R, R¹ and R³ is a Z group
esters of sulfosuccinic acid or of sulfosuccinates of the general formula (T-III):

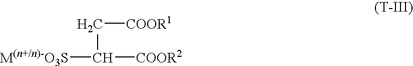

in which the M^{(n+/n)}, for n=1, is a hydrogen atom, an alkali metal cation, an ammonium group or the cation of an ammonium-organic base; and for n=2, is an alkaline earth metal cation and R¹ and R², independently of each other, are a hydrogen atom, an alkali or alkaline earth metal cation, an ammonium group, the cation of an ammonium-organic base or a group Z which is derived from a polyhydroxylated organic compound selected from the group of etherified (C₆-C₁₆) alkyl-polysaccharides with 1 to 6 monomeric saccharide units and/or the etherified (C₆-C₁₆) hydroxyalkyl polyols with 2 to 16 hydroxyl groups, provided that at least one of the groups R¹ or R² is a group Z.
sulfosuccinic acid monoalkyl and dialkyl esters with 8 to 24 atoms in the alkyl group and sulfosuccinic acid monoalkyl polyoxyethyl esters with 8 to 24 C atoms in the alkyl group and 1 to 6 ethoxy groups
alkyl sulfates and alkyl polyglycol ether sulfates of the formula (R—(O—CH₂—CH₂)ₓ—OSO₃H, in which R is preferably a linear alkyl group with 8 to 30 C atoms and x=0 or 1-12,
esters of tartaric acid and citric acid with alcohols which are addition products of about 2-15 molecules of ethylene oxide and/or propylene oxide to C₈₋₂₂ fatty alcohols,
alkyl and/or alkenyl ether phosphates,
sulfated fatty acid alkylene glycol esters,
monoglyceride sulfates and monoglyceride ether sulfates.

Preferred anionic surfactants and emulsifiers are acyl glutamates, acyl isethionates, acyl sarcosinates and acyl taurates, each with a linear or branched acyl group having 6 to 22 carbon atoms and 0, 1, 2, or 3 double bonds, which in specially preferred embodiments are selected from an octanoyl, decanoyl, lauroyl, myristoyl, palmitoyl and stearoyl group, esters of tartaric acid, citric acid or succinic acid, or the salts of these acids with alkylated glucose, especially the products having the INCI designation of Disodium Coco-glucoside citrate, Sodium Coco-glycoside tartrate and Disodium Coco-glucoside sulfosuccinate, alkyl polyglycol ether sulfates and ether carboxylic acids with 8 to 18 C atoms in the alkyl group and up to 12 ethoxy groups in the molecule, sulfosuccinic acid monoalkyl and dialkyl esters with 8 to 18 C atoms in the alkyl group and sulfosuccinic acid monoalkyl polyoxyethyl esters with 8 to 18 C atoms in the alkyl group and 1 to 6 ethoxy groups.

Those surface-active compounds that have at least one quaternary ammonium group and at least one —COO(⁻) or —SO₃(⁻) in the molecule are called zwitterionic surfactants and emulsifiers. Especially suitable zwitterionic surfactants and emulsifiers are the so-called betaines such as N-alkyl-N,N-dimethylammonium glycinates, such as cocoalkyl dimethylammonium glycinate, N-acyl-aminopropyl-N,N-dimethylammonium glycinate, such as cocoacyl aminopropyl dimethylammonium glycinate, and 2-alkyl-3-carboxymethyl-3-hydroxyethylimidazolines, each with 8 to 18 C atoms in the alkyl or acyl group, and cocoacylaminoethyl hydroxyethyl carboxymethyl glycinate. One preferred zwitterionic surfactant is the fatty acid amide derivative known by the INCI designation of Cocamidopropyl betaine.

The term ‘ampholytic surfactants and emulsifers’ is understood to mean those surface-active compounds that have, aside from a C₈-C₂₄ alkyl or acyl group, at least one free amino group and at least one —COOH or —SO₃H group, and which are able to form internal salts. Examples of suitable ampholytic surfactants include N-alkylglycines, N-alkylaminopropionic acids, N-alkylaminobutyric acids, N-alkylimino dipropionic acids, N-hydroxyethyl-N-alkylamidopropyl glycines, N-alkyltaurines, N-alkylsarcosines, 2-alkylaminopropionic acids and alkylaminoacetic acids, each with about 8 to 24 C atoms in the alkyl group. Especially preferred ampholytic surfactants are N-cocoalkylaminopropionate, cocoacylaminoethylaminopropionate, and C₁₂-C₁₈-acylsarcosine.

Nonionic surfactants and emulsifiers contain, as hydrophilic groups, for example, a polyol group, a polyalkyleneglycol ether group or a combination of polyol and polyglycolether groups. Examples of such compounds include
addition products of 2 to 50 moles of ethylene oxide and/or 0 to 5 moles of propylene oxide to linear and branched fatty acids with 8 to 30 C atoms, to fatty acids with 8 to 30 C atoms and to alkylphenols with 8 to 15 C atoms in the alkyl group,
C₁₂-C₃₀ fatty acid monoesters and diesters of addition products of 1 to 30 moles of ethylene oxide to polyols with 3 to 6 carbon atoms, especially to glycerin,
addition products of 5 to 60 moles of ethylene oxide to castor oil and to hydrogenated castor oil,
polyol fatty acid (partial) esters, such as Hydagen® (Cognis) or Sovermol® types (Cognis), especially of saturated C₈₋₃₀ fatty acids,
alkoxylated triglycerides,
alkoxylated fatty acid alkyl esters,
amine oxides,
fatty acid alkanolamides, fatty acid N-alkyl glucamides and fatty amines, as well as their ethylene oxide or polyglycerol addition products,
sorbitan fatty acid esters and addition products of ethylene oxide to sorbitan fatty acid esters, such as polysorbates,
sugar fatty acid esters and methylglucoside fatty acid esters, as well as their ethylene oxide or polyglycerol addition products,
alkyl polyglycosides corresponding to the general formula RO-(Z)ₓ, where R stands for alkyl, Z for sugar, and x for the number of sugar units,

Those alkyl polyglycosides are preferred in which R comprises
essentially C₈ and C₁₀ alkyl groups
essentially C₁₂ and C₁₄ alkyl groups
essentially C₈ to C₁₆ alkyl groups or
essentially C₁₂ to C₁₆ alkyl groups or
essentially C₁₆ to C₁₈ alkyl groups.

Any desired monosaccharide or oligosaccharide can be used as the sugar component Z. Usually, sugars with 5 or 6 carbon atoms and the corresponding oligosaccharides are used. Examples of such sugars include glucose, fructose, galactose, arabinose, ribose, xylose, lyxose, allose, altrose, mannose, gulose, idose, talose and sucrose. Glucose, fructose, galactose, arabinose and sucrose are preferred sugar components. Glucose is especially preferred.

The alkyl polyglycosides that can be used according to the invention comprise, on the average, 1.1 to 5 sugar units. Alkyl polyglycosides with x values of 1.1 to 2.0 are preferred nonionic surfactants. Alkyl glycosides in which x is 1.1 to 1.8 are very specially preferred.
mixtures of alkyl-(oligo)-glucosides and fatty alcohols, e.g., Montanov®68,
sterols, e.g., ergosterol, stigmasterol, sitosterol and mycosterol,
phospholipids, e.g., lecithins or phosphatidyl cholines,
polyglycerol and polyglycerol derivatives such as polyglycerol-poly-12-hydroxystearate (Dehymuls® PGPH) or triglycerol diisostearate (Lameform® TGI,
   alkoxylated polydialkylsiloxanes (INCI designation: Dimethicone polyol).

The alkyl polyglycosides, optionally mixed with fatty alcohols, alkoxylated polydialkylsiloxanes, products of alkylene oxide addition to saturated linear fatty alcohols and fatty acids, each having 2 to 30 moles of ethylene oxide per mole of fatty alcohol or fatty acid.

Cationic surfactants of the quaternary ammonium type, the esterquats and amidoamines are also usable according to the invention. Preferred quaternary ammonium compounds include ammonium halides, especially chlorides and bromides, such as alkyl-trimethylammonium chloride, dialkyldimethylammonium chloride and trialkylmethylammonium chloride. The long alkyl chains of these surfactants preferably have 10 to 18 carbon atoms, such as in cetyltrimethylammonium chloride, stearyltrimethylammonium chloride, distearyidimethylammonium chloride, lauryldimethylammonium chloride, lauryldimethylbenzylammonium chloride and tricetylmethylammonium chloride. The imidazolium compounds known by their INCI designations as Quaternium-27 and Quaternium-83 are other preferred cationic surfactants.

Those agents are quite specially preferred according to the invention which also comprise fatty alcohol(s) and/or fatty alcohol alkoxylate(s), preferably C₁₂₋₂₂-fatty alcohol(s) and/or C₁₂₋₂₂-fatty alcohol ethoxylate(s) with 10 to 30 ethylene oxide units. C₁₆₋₁₈-fatty alcohol(s) and/or C₁₆₋₁₈-fatty alcohol ethoxylates with 12 to 20 ethylene oxide units are specially preferred, preferably in proportions of 5 to 20% by weight, preferably 7.5 to 17.5% by weight, and particularly 10 to 15% by weight, based in each case on the weight of the agent.

Summarizing, hair treatment agents according to the invention preferably comprise also amphoteric surfactant(s), preferably from the groups of the
N-alkylglycines,
N-alkylpropionic acids,
N-alkylaminobutyric acids,
N-alkyliminodipropionic acids,
N-hydroxyethyl-N-alkylamidopropylglycines,
N-alkyltaurines,
N-alkylsarcosines,
2-alkylaminopropionic acids with about 8 to 24 C atoms in the alkyl group
alkylaminoacetic acids with about 8 to 24 C atoms in the alkyl group,
N-cocoalkylaminopropionate,
C₁₂-C₁₈-acylsarcosine,
N-alkyl-N,N-dimethylammonium glycinate, such as cocoalkyl-dimethylammonium glycinate,
N-acylaminopropyl-N,N-dimethylammonium glycinate, such as cocoacyl-aminopropyldimethylammonium glycinate,
2-alkyl-3-carboxymethyl-3-hydroxyethylimidazoline with 8 to 18 C atoms in the alkyl or acyl groups,
cocoacylaminoethyl-hydroxyethyl-carboxymethylglycinate,
the compounds known by the INCI designation Cocamidopropyl betaine,
the compounds known by the INCI designation Disodium cocoamphodiacetate,
   with preferred agents comprising the amphoteric surfactant(s) in proportions of 1 to 15% by weight, preferably 2.5 to 12% by weight, and especially 5 to 10% by weight, based in each case on the total agent.

As other optional components, the agents according to the invention can comprise 0.01 to 10% by weight of at least one polymer from the group of cationic and/or amphoteric polymers.

Cationic or amphoteric polymers are understood to be polymers that have, in the main chain and/or side chain a group that can be “temporarily” or “permanently” cationic. Those polymers that have a cationic group independently of the pH of the agent are called “permanently cationic” according to the invention. As a rule, these are polymers comprising a quaternary nitrogen atom, in the form of an ammonium group, for example. Quaternary ammonium groups are preferred cationic groups. Those polymers in which the quaternary ammonium group is bound through a C₁₋₄ hydrocarbon group to a main polymer chain built up of acrylic acid, methacrylic acid or their derivates have proved to be particularly suitable.

In addition to, or in place of, the cationic polymers, the agent according to the invention can also contain amphoteric polymers. These also have at least one negatively charged group in the molecule, and are also called zwitterionic polymers.

The polymer or polymers are preferably used within narrow quantitative ranges. Thus agents are preferred according to the invention that comprise, based on its weight, 0.05 to 7.5% by weight, preferably 0.1 to 5% by weight, especially preferably 0.2 to 3.5% by weight, and particularly 0.25 to 2.5% by weight amphoteric polymer(s).

Independently of whether or not the agents comprise amphoteric polymers, other preferred agents according to the invention are distinguished by comprising, based on its weight, 0.05 to 7.5% by weight, preferably 0.1 to 5% by weight, especially preferably 0.2 to 3.5% by weight, and particularly 0.25 to 2.5% by weight cationic polymer(s).

Cationic polymers preferred for use according to the invention are described below:

Homopolymers of the general formula (G1-I),

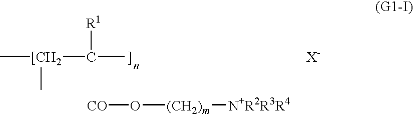

in which R¹ is —H or —CH3, R², R³, and R⁴, independently of each other, are C₁₋₄ alkyl, alkenyl, or hydroxyalkyl groups, m is 1, 2, 3, or 4, n is a natural number, and X— is a physiologically acceptable organic or inorganic anion, and copolymers consisting essentially of the monomer units introduced in formula (G1-I) and nonionic monomer units are specially preferred cationic polymers. Of these polymers, those are preferred according to the invention for which at least one of the following conditions is true:
R¹ stands for a methyl group
R², R³, and R⁴ stand for methyl groups
m has the value of 2.

Examples of physiologically acceptable counterions X— include halide ions, sulfate ions, phosphate ions, methosulfate ions and organic ions such as lactate, citrate, tartrate and acetate ions. Halide ions are preferred, especially chloride.

One specially suitable homopolymer is the, optionally crosslinked, poly(methacryloyloxyethyltrimethylammonium chloride) with the INCI designation Polyquaternium-37. Such products are commercially available, for instance, under the names Rheocare® CTH, (Cosmetic Rheologies), and Synthalen® CR (Ethnichem). The crosslinking can if desired be accomplished using multiply olefinically unsaturated compounds such as divinylbenzene, tetraallyloxyethane, methylene bisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylene bisacrylamide is a preferred crosslinking agent.

The homopolymer is preferably used in the form of a nonaqueous polymer dispersion that should have a polymer concentration not less than 30% by weight. Such polymer dispersions are commercially available under the names Salcare® SC 95 (ca. 50% polymer; other components: mineral oil (INCI designation: Mineral oil) and tridcecyl-polyoxypropylene-polyoxyethylene ether (INCI designation: PPG-1-Trideceth-6)) and Salcare® 960 (ca. 50% polymer, other components: mixture of diesters of propylene glycol with a mixture of caprylic and capric acid (INCI designation: Propylene glycol dicaprylate/dicaprate) and tridecyl-polyoxypropylene-polyoxyethylene ether (INCI designation: PPG-1-Trideceth-6).

Copolymers with monomer units according to formula (G1-I) comprise, as the nonionic monomer units, preferably acrylamide, methacrylamide, acrylic acid C₁₋₄-alkyl esters and methacrylic acid C₁₋₄-alkyl esters. Of these nonionic monomers, acrylamide is specially preferred. These copolymers can also be crosslinked, as in the case of the homopolymers described above. One copolymer preferred according to the invention is the crosslinked acrylamide-methacryloyloxyethyltrimethylammonium chloride copolymer. Such copolymers in which the monomers occur in a weight ratio of about 20:80 are commercially available as the approximately 50% nonaqueous polymer dispersion under the designation Salcare® SC 92.

Examples of other preferred cationic polymers are
quaternized cellulose derivatives, such as those commercially available under the designations Celquat® and Polymer JR®. The compounds Celquat® H 100, Celquat® L 200 and Polymer JR® 400 are preferred quaternized cellulose derivatives
quaternized alkyl polyglycosides as described in German Patent 44 13 686,
cationized honey, such as the commercial product Honeyquat® 50,
cationized guar derivatives, particularly such as the products marketed under the trade names Cosmedia® Guar and Jaguar®,
polymeric dimethyldiallylammonium salts and their copolymers with ethers and amides of acrylic acid and methacrylic acid. The products commercially available under the tradenames Merquat® 100 (poly(dimethyldiallylammonium chloride)) and Merquat® 550 (dimethyldiallylammonium chloride-acrylamide copolymer) are examples of such cationic polymers.
copolymers of vinylpyrrolidone with quaternized derivatives of dialkyl-aminoalkylacrylates and dialkylaminoalkylmethacrylates. such as the vinylpyrrolidone-dimethylaminoethylmethacrylate copolymers quaternized with diethyl sulfate. Such compounds are commercially available under the tradenames Gafquat® 734 and Gafquat® 755.
vinylpyrrolidone-vinylimidazolium methochloride copolymers, such as those offered under the tradenames Luviquat® FC 370, FC 550, FC 905 and HM 552,
quaternized polyvinyl alcohol,
and the polymers with quaternary nitrogen atoms in the main polymer chain, known by the tradenames Polyquaternium 2, Polyquaternium 17, Polyquaternium 18 and Polyquaternium 27.

The polymers known by the tradename Polyquaternium 24 (commercial product, e.g., Quatrisoft® LM 200) can equally well be used as cationic polymers. Likewise usable according to the invention are the copolymers of vinylpyrrolidone, such as those available as the commercial products Copolymer 845 (Manufacturer: ISP), Gaffix® VC 713 (Manufacturer: ISP), Gafquat® ASCP 1011, Gafquat® HS 110 Luviquat® 8155 and Luviquat® MS 370.

Cationic protein hydrolyzates can also be used as cationic polymers. Preferred agents comprise one or more cationic protein hydrolyzates from the group of cocodimonium hydroxypropyl hydrolyzed collagen, cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed soy protein, cocodimonium hydroxypropyl hydrolyzed wheat protein, hydroxypropyl hydrolyzed wheat protein, hydroxypropyl arginine lauryl/myristyl ether HCl, hydroxypropyltrimonium gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratin, hydroxypropyltrimonium hydrolyzed rice bran protein, hydroxypropyltrimonium hydrolyzed soy protein, hydroxypropyltrimonium hydrolyzed vegetable protein, hydroxypropyltrimonium hydrolyzed wheat protein, hydroxypropyltrimonium hydrolyzed what protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, laurdimonium hydroxypropyl hydrolyzed casein, laurdimonium hydroxypropyl hydrolyzed collagen, laurdimonium hydroxypropyl hydrolyzed keratin, laurdimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratin, steardimonium hydroxypropyl hydrolyzed rice protein, steardimonium hydroxypropyl hydrolyzed soy protein, steardimonium hydroxypropyl hydrolyzed vegetable protein, steardimonium hydroxypropyl hydrolyzed wheat protein, steardimonium hydroxypropyl hydrolyzed collagen, Quaternium-76-hydrolyzed collagen, Quaternium-79-hydrolyzed collagen, Quaternium-79-hydrolyzed keratin, Quaternium-79-hydrolyzed milk protein, Quaternium-79-hydrolyzed soy protein, Quaternium-79-hydrolyzed wheat protein.

Summarizing, those hair treatment agents are preferred according to the invention that comprise, based on its weight, 0.05 to 7.5% by weight, preferably 0.1 to 5% by weight, especially preferably 0.2 to 3.5% by weight and particularly 0.25 to 2.5% by weight cationic polymer(s), with the preferred cationic polymer(s) selected from:
a. poly(methacryloyloxyethyltrimethylammonium chloride) (INCI: Polyquaternium-37 and/or
b. quaternized cellulose derivatives (INCI: Polyquaternium 10) and/or
c. cationic alkyl polyglycosides and/or
d. cationized honey and/or
e. cationic guar derivatives and/or
f. polymeric dimethyldiallylammonium salts and their copolymers with esters and amides of acrylic acid and methacrylic acid and/or
g. copolymers of vinylpyrrolidone with quaternized derivatives of dialkylaminoalkyl acrylate and dialkylaminoalkyl methacrylate and/or
h. vinylpyrrolidone-vinylimidazolium methochloride copolymers and/or
i. quaternized polyvinyl alcohol and/or
j. Polyquaternium 2 and/or
k. Polyquaternium-7 and/or
l. Polyquaternium 17 and/or
m. Polyquaternium 18 and/or
n. Polyquaternium 24 and/or
o. Polyquaternium 27.

The agents according to the invention can contain amphoteric polymers instead of or in addition to the cationic polymers. Amphoteric polymers that are preferably usable in the framework of the present invention are made up essentially of

A) monomers with quaternary ammonium groups of the general formula (Z-I).

R¹—CH═CR²—CO-Z-(CₙH₂ₙ)—N⁽⁺⁾R³R⁴R⁵A⁽⁻⁾ (Z-I)

in which R¹ and R², independently of each other, stand for hydrogen or a methyl group and R³, R⁴, and R⁵, independently of each other, stand for alkyl groups with 1 to 4 carbon atoms, Z is a NH group or an oxygen atom, n is an integer from 2 to 5 and A⁽⁻⁾ is the anion of an organic or inorganic acid
and
B) monomeric carboxylic acids of the general formula (Z-II),

R⁶—CH═CR⁷—COOH (z-II)

in which R⁶ and R⁷, independently of each other, are hydrogen or methyl groups.

Examples of suitable starting monomers include dimethylaminoethylacrylamide, dimethylaminoethylmethacrylamide, dimethylaminopropylacrylamide, dimethylaminopropylmethacrylamide and diethylaminoethylacrylamide, if Z means a NH group, or dimethylaminoethyl acrylate, dimethylaminoethyl methacrylate and dimethylaminoethyl acrylate, if Z is an oxygen atom.

The monomers containing a tertiary amino group are then quaternized in the known manner, with methyl chloride, dimethyl sulfate or diethyl sulfate especially suitable. The quaternization reaction can be carried out in aqueous solution or in the solvent.

Those monomers of the formula (Z-I) that are derivatives of acrylamide or methacrylamide are advantageous. Those monomers containing halide, methoxysulfate or ethoxysulfate ions as the counterions are further preferred. Those monomers of the formula (Z-I) in which R³, R⁴, and R⁵ are methyl groups are likewise preferred.

Acrylamidopropyl-trimethylammonium chloride is a quite specially preferred monomer of formula (Z-I).

Suitable monomeric carboxylic acids of formula (Z-II) include acrylic acid, methacrylic acid, crotonic acid and 2-methylcrotonic acid. Acrylic acid or methacrylic acid, especially acrylic acid, are used preferably.

The zwitterionic polymers usable according to the invention are produced from monomers of formulas (Z-I) and (Z-II) by polymerization processes that are themselves known. The polymerization can be done in either aqueous or aqueous-alcoholic solution. Alcohols with 1 to 4 carbon atoms are used as the alcohols, preferably isopropanol. They act simultaneously as polymerization controllers. However, other components may also be added to the monomer solution as controllers, such as formic acid, or mercaptans such as thioethanol and thioglycolic acid. The polymerization is initiated with radical-forming substances. Redox systems and/or thermally decomposing radical formers of the azo compound type, such as azoisobutronitrile, azo-bis-(cyanopentanoic acid) or azo-bis-(amidinopropane) dihydrochloride can be used for this purpose. Suitable redox systems are, for example, combinations of hydrogen peroxide, potassium or ammonium peroxodisulfate and tertiary-butyl hydroperoxide with sodium sulfite, sodium dithionite or hydroxylamine hydrochloride as reducing components.

The polymerization can be done isothermally or under adiabatic conditions. Depending on the concentration relations, the liberated heat of polymerization may cause to temperature to vary within the course of the reaction from 20 to 200° C., and the reaction must optionally be carried out under the self-generated overpressure. The reaction temperature is preferably between 20 and 100° C.

The pH during the copolymerization can vary over a wide range. It is advantageous to do the polymerization at low pH, but pH values higher than the neutral point are also possible. After the polymerization, a pH between 5 and 10, preferably 6 to 8, is adjusted with an aqueous base such as sodium hydroxide, potassium hydroxide or ammonia. More detailed information on the polymerization conditions can be found from the examples.

Those polymers in which the monomers of formula (Z-I) are in excess over the monomers of formula (Z-II) have proved to be especially effective. Thus it is preferred according to the invention to use those polymers that comprise monomers of formulas (Z-I) and (Z-II) in a molar ratio of 60:40 to 95:5, particularly 75:25 to 95:5.

Agents preferred according to the invention are characterized by comprising, in the amphoteric polymer(s), monomers A) and B), with A) and B) selected from

A) monomers with quaternary ammonium groups of the general formula (Z-I),

R¹—CH═CR²—CO-Z-(CₙH₂ₙ)—N⁽⁺⁾R³R⁴R⁵A⁽⁻⁾ (Z-I)
in which the R¹ and R², independently of each other, stand for hydrogen or a methyl group and R³, R⁴, and R⁵, independently of each other, stand for alkyl groups with 1 to 4 carbon atoms, Z is a NH group or an oxygen atom, n is an integer from 2 to 5 and A⁽⁻⁾ is the anion of an organic or inorganic acid
   and
   B) monomeric carboxylic acids of the general formula (Z-II).

R⁶—CH═CR⁷—COOH (Z-II)
in which the R⁶ and R⁷, independently of each other, stand for hydrogen or methyl groups.

It is especially preferable for the amphoteric polymers used in the agents according to the invention to comprise monomers from the group of acrylamides and/or methacrylamides with alkylammonium groups. Acrylic acid and/or methacrylic acid and/or crotonic acid and/or 2-methylcrotonic acid have also proven themselves as additional monomers with anionic groups comprising the polymers.

Summarizing, agents are preferred according to the invention that comprise in the amphoteric polymer(s) at least one of the monomers
trimethylammonium ethylacrylamide and/or
trimethylammonium ethylmethacrylamide and/or
trimethylammonium propylacrylamide and/or
trimethylammonium propylmethacrylamide and/or
trimethylammonium ethylacrylamide and/or
trimethylammonium ethyl acrylate and/or
trimethylammonium ethyl methacrylate and/or
trimethylammonium ethyl acrylate and/or
ethyldimethylammonium ethyl acrylamide and/or
ethyldimethylammonium ethyl methacrylamide and/or
ethyldimethylammonium propyl acrylamide and/or
ethyldimethylammonium propyl methacrylamide and/or
ethyldimethylammonium ethyl acrylamide and/or
ethyldimethylammonium ethyl acrylate and/or
ethyldimethylammonium ethyl methacrylate and/or
ethyldimethylammonium ethyl acrylate
   with at least one of the monomers
acrylic acid and/or
methacrylic acid and/or
crotonic acid and/or
2-methylcrotonic acid.

Amphoteric polymers specially preferred according to the invention are:
copolymers of trimethylammonium ethyl acrylamide with acrylic acid
copolymers of trimethylammonium ethyl acrylamide with methacrylic acid
copolymers of trimethylammonium ethyl acrylamide with crotonic acid
copolymers of trimethylammonium ethyl acrylamide with 2-methyl-crotonic acid
copolymers of trimethylammonium ethyl methacrylamide with acrylic acid
copolymers of trimethylammonium ethyl methacrylamide with methacrylic acid
copolymers of trimethylammonium ethyl methacrylamide with crotonic acid
copolymers of trimethylammonium ethyl methacrylamide with 2-methylcrotonic acid
copolymers of trimethylammonium ethyl methacrylamide with
copolymers of trimethylammonium propyl acrylamide with acrylic acid
copolymers of trimethylammonium propyl acrylamide with methacrylic acid
copolymers of trimethylammonium propyl acrylamide with crotonic acid
copolymers of trimethylammonium propyl acrylamide with 2-methylcrotonic acid
copolymers of trimethylammonium propyl methacrylamide with acrylic acid
copolymers of trimethylammonium propyl methacrylamide with methacrylic acid
copolymers of trimethylammonium propyl methacrylamide with crotonic acid
copolymers of trimethylammonium propyl methacrylamide with 2-methylcrotonic acid
copolymers of trimethylammonium ethyl acrylamide with acrylic acid
copolymers of trimethylammonium ethyl acrylamide with methacrylic acid
copolymers of trimethylammonium ethyl acrylamide with crotonic acid
copolymers of trimethylammonium ethyl acrylamide with 2-methylcrotonic acid
copolymers of trimethylammonium ethyl acrylate with acrylic acid
copolymers of trimethylammonium ethyl acrylate with methacrylic acid
copolymers of trimethylammonium ethyl acrylate with crotonic acid
copolymers of trimethylammonium ethyl acrylate with 2-methylcrotonic acid
copolymers of trimethylammonium ethyl methacrylate with acrylic acid
copolymers of trimethylammonium ethyl methacrylate with methacrylic acid
copolymers of trimethylammonium ethyl methacrylate with crotonic acid
copolymers of trimethylammonium ethyl methacrylate with 2-methylcrotonic acid
copolymers of trimethylammonium ethyl acrylate with acrylic acid
copolymers of trimethylammonium ethyl acrylate with methacrylic acid
copolymers of trimethylammonium ethyl acrylate with crotonic acid
copolymers of trimethylammonium ethyl acrylate with 2-methylcrotonic acid
copolymers of ethyldimethylammonium ethyl acrylamide with acrylic acid
copolymers of ethyldimethylammonium ethyl acrylamide with methacrylic acid
copolymers of ethyldimethylammonium ethyl acrylamide with crotonic acid
copolymers of ethyldimethylammonium ethyl acrylamide with 2-methylcrotonic acid
copolymers of ethyldimethylammonium ethyl methacrylamide with acrylic acid
copolymers of ethyldimethylammonium ethyl methacrylamide with methacrylic acid
copolymers of ethyldimethylammonium ethyl methacrylamide with crotonic acid
copolymers of ethyldimethylammonium ethyl methacrylamide with 2-methylcrotonic acid
copolymers of ethyldimethylammonium propyl acrylamide with acrylic acid
copolymers of ethyldimethylammonium propyl acrylamide with methacrylic acid
copolymers of ethyldimethylammonium propyl acrylamide with crotonic acid
copolymers of ethyldimethylammonium propyl acrylamide with 2-methylcrotonic acid
copolymers of ethyldimethylammonium propyl methacrylamide with acrylic acid
copolymers of ethyldimethylammonium propyl methacrylamide with methacrylic acid
copolymers of ethyldimethylammonium propyl methacrylamide with crotonic acid
copolymers of ethyldimethylammonium propyl methacrylamide with 2-methylcrotonic acid
copolymers of ethyldimethylammonium ethyl acrylamide with acrylic acid
copolymers of ethyldimethylammonium ethyl acrylamide with methacrylic acid
copolymers of ethyldimethylammonium ethyl acrylamide with crotonic acid
copolymers of ethyldimethylammonium ethyl acrylamide with 2-methylcrotonic acid
copolymers of ethyldimethylammonium ethyl acrylate with acrylic acid
copolymers of ethyldimethylammonium ethyl acrylate with methacrylic acid
copolymers of ethyldimethylammonium ethyl acrylate with crotonic acid
copolymers of ethyldimethylammonium ethyl acrylate with 2-methylcrotonic acid
copolymers of ethyldimethylammonium ethyl methacrylate with acrylic acid
copolymers of ethyldimethylammonium ethyl methacrylate with methacrylic acid
copolymers of ethyldimethylammonium ethyl methacrylate with crotonic acid
copolymers of ethyldimethylammonium ethyl methacrylate with 2-methylcrotonic acid
copolymers of ethyldimethylammonium ethyl acrylate with acrylic acid
copolymers of ethyldimethylammonium ethyl acrylate with methacrylic acid
copolymers of ethyldimethylammonium ethyl acrylate with crotonic acid
copolymers of ethyldimethylammonium ethyl acrylate with 2-methylcrotonic acid

The cosmetic agents according to the invention can further comprise all the active substances, additives and aids known for such preparations. In many cases the agents contain at least one surfactant, with not only anionic but also zwitterionic, ampholytic, nonionic and cationic surfactants suitable in principle. But in many cases is has proven to be advantageous to select the surfactants from anionic, zwitterionic or nonionic surfactants. These surfactants are described in detail above.

In a further preferred embodiment the agents according to the invention can comprise emulsifiers (F). Emulsifiers cause development of water-stable or oil-stable adsorption layers at the phase interfaces. Those protect the dispersed droplets from coalescence and so stabilize the emulsion. Emulsifiers, like surfactants, are made up of a hydrophobic part of the molecule and a hydrophilic part of the molecule. Hydrophilic emulsifiers preferably form O/W emulsions and hydrophobic emulsifiers preferably form W/O emulsions. An emulsion is understood to mean a droplet-like distribution (dispersion) of one liquid in another liquid, with use of energy to provide stabilizing interphase boundaries by means of surfactants. The selection of these emulsifying surfactants or emulsifiers is determined according to the substances to be dispersed and the other phase, as well as the fineness of the emulsion.

Examples of emulsifiers usable according to the invention include addition products of 4 to 30 moles of ethylene oxide and 0 to 5 moles of propylene oxide to linear fatty alcohols with 8 to 22 C atoms, to fatty acids with 12 to 22 C atoms, and to alkylphenols with 8 to 15 C atoms in the alkyl group,
C₁₂-C₂₂-fatty acid monoesters and diesters of products of addition of 1 to 30 moles of ethylene oxide to polyols with 3 to 6 carbon atoms, especially to glycerol,
products of addition of ethylene oxide and polyglycerol to methylglucoside fatty acid esters, fatty acid alkalolamides and fatty acid glucamides,
C₈-C₂₂-alkylkmonoglycosides and C₁₂-C₂₂-alkyloligoglycosides and their ethoxylated analogs, with degrees of oligomerization of 1.1 to 5 preferred, especially 1.2 to 2.0, and with glucose preferred as the sugar component,
mixtures of alkyl-(oligo)-glucosides and fatty alcohols, such as the commercially available product Montanov® 68,
products of addition of 5 to 60 moles of ethylene oxide to castor oil and hydrogenated castor oil,
partial esters of polyols having 3-6 carbon atoms with saturated fatty acids having 8 to 22 C atoms,
sterols. Sterols are understood to be a group of steroids having a hydroxyl group at carbon atom 3 of the steroid structure. They are isolated both from animal tissues (zoosterols) and from plant fats (phytosterols). Cholesterol and lanosterol are examples of zoosterols. Ergosterol, stigmasterol and sitosterol are examples of suitable physosterols. Sterols, called mycosterols, are also isolated from molds and yeasts.
phospholipids. These are understood to be primarily the glucose phospholipids, which are obtained, for example, as lecithins or phosphatidylcholines from egg yolks, for instance, or from plant seeds (such as soybeans).
fatty acid esters of sugars and sugar alcohols, such as sorbitol,
polyglycerol and polyglycerol derivatives such as polyglycerol-poly-12-hydroxystearate (commercial product Dehymuls® PGPH),
linear and branched fatty acids with 8 to 30 C atoms and their sodium, potassium, ammonium, calcium, magnesium and zinc salts.

The agents according to the invention preferably contain the emulsifiers in proportions of 0.1 to 25% by weight, especially 0.5-15% by weight, based on the complete agent.

The compositions according to the invention can preferably comprise at least one nonionic emulsifier with a HLB [hydrophile-lipophile balance] value of 8 to 18. Nonionic emulsifiers with a HLB value of 10-15 can be especially preferable according to the invention.

It has further been found advantageous for the agents according to the invention to comprise other polymers (G) in addition to the polymer(s) from the group of cationic and/or amphoteric polymers. In a preferred embodiment, therefore, other polymers are added to the agents according to the invention. Both anionic and nonionic polymers have proved to be effective.

The anionic polymers (G2) are anionic polymers with carboxylate and/or sulfonate groups. Examples of anionic monomers of which such polymers may consist include acrylic acid, methacrylic acid, crotonic acid, maleic acid anhydride and 2-acrylamido-2-methylpropanesulfonic acid. The acidic groups can occur partially or entirely as the sodium, potassium, ammonium, monoethanolammonium or triethanolammonium salts. Preferred monomers are 2-acrylamido-2-methylpropanesulfonic acid and acrylic acid.

Those anionic polymers have proved very specially effective which comprise, alone or as a comonomer, 2-acrylamido-2-methylpropanesulfonic acid, in which the sulfonic acid group can be partially or entirely as the sodium, potassium, ammonium, monoethanolammonium or triethanolammonium salt.

The homopolymer of 2-acrylamido-2-methylpropanesulfonic acid is particularly preferred. It is commercially available, for instance, under the tradename Rheothik® 11-80.

In this embodiment it can be preferred to use copolymers of at least one anionic monomer and at least one nonionic monomer. Refer to the substances listed above for the anionic monomers. Preferred nonionic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers and especially copolymers of polyacrylamide with monomers containing sulfonic acid groups. One particularly preferred anionic copolymer comprises 70 to 55 mole-% acrylamide and 30 to 45 mole-% 2-acrylamido-2-methylpropane-sulfonic acid, in which the sulfonic acid groups occur partially or completely as sodium, ammonium, monoethanolammonium or triethanolammonium salts. This copolymer can also be crosslinked, preferably using polyolefinically unsaturated compounds such as tetraallyloxyethane, allylsucrose, allylpentaerythritol and methylene-bis-acrylamide as crosslinking agents. The commercial produce Sepigel® 305 from the company SEPPIC comprises such a polymer. Use of this product, which contains a mixture of hydrocarbons (C₁₃-C₁₄)-isoparaffins) and a nonionic emulsifier (Laureth-7) along with the polymer component, has proven particularly advantageous within the structure of the teaching of the invention.

Sodium acryloyl-dimethyl taurate copolymers marketed as mixed products with isohexadecane and Polysorbate-80 under the tradename of Simulgel® 600 have also proven especially effective according to the invention.

Crosslinked and uncrosslinked polyacrylic acids are likewise preferred anionic homopolymers. Allyl ethers of pentaerythritol, of sucrose, and of propylene can be preferred crosslinking agents. Such compounds are commercially available, for example, under the tradename Carbopol®.

Copolymers of maleic anhydride and methyl vinyl ether, especially those with crosslinks, are likewise color-retaining polymers. A maleic anhydride -methyl vinyl ether copolymer crosslinked with 1,9-decadiene is commercially available under the name of Stabileze® QM.

In a further embodiment, the agents according to the invention can contain nonionic polymers (G4).

### Examples of suitable nonionic polymers are

vinylpyrrolidone/vinyl ester copolymers. such as are marketed under the tradenames Luviskol® (BASF). Luviskol® VA 64 and Luviskol® VA 73, both vinylpyrrolidone/vinyl acetate copolymers, are likewise preferred nonionic polymers.
cellulose ethers, such as hydroxypropylcellulose, hydroxyethylcellulose and methylhydroxypropylcellulose, such as are marketed under the tradenames Culminal® and Benecel® (AQUALON) and Natrosol® types (Hercules).
starches and their derivatives, especially starch ethers, such as Structure® XL (National Starch), a multifunctional salt-tolerant starch;
shellac
polyvinylpyrrolidones, such as are marketed under the tradename of Luviskol® (BASF),
siloxanes. These siloxanes can be water-soluble as well as water-insoluble. Both volatile and non-volatile siloxanes are suitable. Non-volatile siloxanes are understood to be those compounds having boiling points above 200° C. at standard pressure. Preferred siloxanes are polydialkylsiloxanes, polyalkyl-arylsiloxanes, such as polyphenylmethylsiloxane, ethoxylated polydialkylsiloxane and polydialkylsiloxanes containing amine and/or hydroxy groups.
glycosidically substituted silicones.

It is also possible, according to the invention, for the preparations to comprise several, especially two, different polymers of the same charge and/or both an ionic and an amphoteric and/or non-ionic polymer.

The further polymers (G) are preferably used in the agents according to the invention at proportions of 0.05 to 10% by weight, based on the total agent. Proportions of 0.1 to 5, and especially of 0.1 to 3% by weight. Proportions of 0.1 to 5, and especially of 0.1 to 3% by weight, are especially preferred.

Furthermore, in a preferred embodiment of the invention, an agent according to the invention can also comprise UV filters (I). The UV filters to be used according to the invention are not subject to general limitations with respect to their structure and their physical properties. Instead, all the UV filters usable in the cosmetic field that have absorption maxima in the UVA (315400 nm), in the UVB (280-315 nm) or in the UVC (<280 nm) are suitable. UV filters having an absorption maximum in the UVB range, especially in the range from about 280 to about 300 nm, are especially preferred.

The UV filters to be used according to the invention can, for example, be selected from substituted benzophenones, p-aminobenzoic acid esters, diphenylacrylic acid esters, cinnamic acid esters, salicylic acid esters, benzimidazoles and o-aminobenzoic acid esters.

Examples of UV filters usable according to the invention include 4-aminobenzoic acid, N,N,N-trimethyl-4-(2-oxoborn-3-ylidenemethyl)methyl sulfate, 3,3,5-trimethylcyclohexyl salicylate (Homosalate), 2-hydroxy-4-methoxybenzophenone (Benzophenone-3; Uvinuyl® M 40, Uvasorb® MET, Neo Heliopan® BB, Eusolex® 4360), 2-phenylbenzimidazole-5-sulfonic acid and its potassium, sodium and triethanolamine salts (phenylbenzimidazole sulfonic acid; Parson® HS; Neo Heliopan® Hydro), 3,3′-(1,4-phenylenedimethylene)-bis-(7,7-dimethyl-2-oxo-bicyclo-[2.2.1]hept-1-yl-methanesulfonic acid) and its salts, 1-(4-tertiary-butylphenyl)-3-(4-methoxyphenyl)-propane-1,3-dione (butyl methoxydibenzoylmethane; Parsol® 1789, Eusolex® 9020), α-(2-oxoborn-3-yliden)toluene-4-sulfonic acid and its salts, ethoxylated 4-aminobenzoic acid ethyl ester (PEG-25 PABA; Uvinul® P 25), 4-dimethylaminobenzoic acid 2-ethylhexyl ester (Octyl Dimethyl PABA; Uvasorb® DMA; Escalol® 507, Eusolex® 6007), salicylic acid 2-ethylhexyl ester (octyl salicylate; Escalol® 587, Neo Heliopan® OS, Uvinul® 18), 4-methoxycinnamic acid isopentyl ester (isoamyl p-methoxycinnamate; Neo Heliopan® E 1000), 4-methoxycinnamic acid 2-ethylhexyl ester (octyl methoxycinnamate; Parsol® MCX, Escalol® 557, Neo Heliopan® AV), 2-hydroxy-4-methoxybenzophenone-5-sulfonic acid and its sodium salt (Benzophenone-4; Uvinul® MS; Uvasorb® S 5), 3-(4′-methylbenzylidene)-D,L-camphor (4-methylbenzylidene camphor; Parsol® 5000, Eusolex® 6300), 3-benzylidenecamphor, 4-isopropylbenzyl salicylate, 2,4,6-trianilino-(p-carbo-2′-ethylhexyl-1′-oxy)-1,3,5-triazine, 3-imidazol-4-yl-acrylic acid and its ethyl ester, polymers of N-{(2 and 4)-2-oxoborn-3-ylidenemethyl]benzyl}acrylamide, 2,4-dihydroxybenzophenone (Benzophenone-1; Uvasorb® 20 H, Uvinul® 400), 1,1′-diphenylacrylonitrile 2-ethylhexyl ester (Octocrylene; Eusolex® OCR, Neo Heliopan® Type 303, Uvinul® N 539 SG), o-aminobenzoic acid menthyl ester (menthyl anthranilate; Neo Heliopan® MA), 2,2′,4,4′-tetrahydroxybenzo-phenone (Benzophenone-2-Uvinul® D-50), 2,2′-dihydroxy-4,4′-dimethoxybenzophenone (Benzophenone-6), 2,2′-dihydroxy-4,4′-dimethoxybenzophenone-5-sulfonic acid sodium salt and 2-cyano-3,3-diphenylacrylic acid 2-ethylhexyl ester. 4-aminobenzoic acid, N,N,N-trimethyl-4-(2-oxoborn-3-ylidenemethyl)aniline methyl sulfate, 3,3,5-trimethylcyclohexyl salicylate, 2-hydroxy-4-methoxybenzophenone, 2-phenylbenzimidazole-5-sulfonic acid and its potassium, sodium and triethanolamine salts, 3,3′-(1,4-phenylenedimethylene)-bis-(7,7-dimethyl-2-oxo-bicyclo-[2.2.1]hept-1-yl-methanesulfonic acid) and its salts, 1-(4-tertiary-butylphenyl)-3-(4-methoxyphenyl)-propane-1,3-dione, α-(2-oxoborn-3-yliden)toluene-4-sulfonic acid and its salts, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxy-benzophenone-5-sulfonic acid and its sodium salt, 3-(4′-methylbenzylidene)-D,L-camphor, 3-benzylidenecamphor, 4-isopropylbenzyl salicylate, 2,4,6-trianilino-(p-carbo-2′-ethylhexyl-1′-oxy)-1,3,5-triazine, 3-imidazol-4-yl-acrylic acid and its ethyl ester, and polymers of N-{(2 and 4)-2-oxoborn-3-ylidenemethyl]benzyl}acrylamide are preferred. 2-hydroxy-4-methoxybenzophenone, 2-phenylbenzimidazol-5-sulfonic acid and its potassium, sodium and triethanolamine salts, 1-(4-tertiary-butylphenyl)-3-(4-methoxyphenyl)-propane-1,3-dione, 4-methoxycinnamic acid 2-ethylhexyl ester and 3-(4′-methylbenzylidene)-D,L-camphor are quite specially preferred.

Those UV filters for which the molar absorptivity at the absorbance maximum is greater than 15,000, preferably greater than 20,000, are preferred.

It has further been found that in the case of structurally similar UV filters, the water-insoluble compound often has the greater effect, within the teaching of the invention, than those water-soluble compounds that differ from it by one or more additional ionic groups. In the invention, those UV filters that are not soluble to more than 1% by weight, and especially not more than 0.1% by weight, at 20° C. are considered water-insoluble. Furthermore these compounds should be soluble to at least 0.1% by weight, preferably at least 1% by weight, at room temperature in the usual cosmetic oil components. Thus it can be preferred according to the invention to use water-insoluble UV filters.

According to a further embodiment of the invention, those UV filters are preferred that have a cationic group, especially a quaternary ammonium group.

These UV filters have the general structure U-Q.

The U part of the structure stands for a group that absorbs UV rays. This group can in principle be derived from the UV filters named above that can be used in the cosmetic field, in which a group, normally a hydrogen atom, of the UV filter is replaced by a quaternary group Q, especially with a quaternary amine function.

Examples of compounds from which the U part of the structure can be derived include:
substituted benzophenones,
p-aminobenzoic acid esters,
diphenylacrylic acid esters,
cinnamic acid esters,
salicylic acid esters,
benzimidazoles and
o-aminobenzoic acid esters.

Structural parts U that are derived from cinnamic acid amide or from N,N-dimethylaminobenzoic acid amide are preferred according to the invention.

The U parts of the structure can in principle be selected so that the absorbance maximum of the UV filter can be in both the UVA range (315-400 nm) and the UVB range (280-315 nm) or in the UV range (<280 nm). UV filters with an absorbance maximum in the UVB range, especially in the range of about 280 to about 300 nm, are specially preferred.

Further, the U part of the structure, also as it depends on the Q part of the structure, is preferably selected so that the molar absorptivity of the UV filter at the absorbance maximum is greater than 15,000, preferably greater than 20,000

The Q part of the structure preferably has a quaternary ammonium group as the cationic group. This quaternary ammonium group can in principle be bonded directly to the U part of the structure, so that the U part of the structure is one of the four substituents of the positively charged nitrogen atom. However, it is preferred for one of the four substituents on the positively charged nitrogen atom to be a group, particularly an alkylene group with 2 to 6 carbon atoms, which acts as the link between the structural part U and the positively charged nitrogen atom.

It is advantageous for the group Q to have the general structure —(CH₂)ₓN+R¹R²R³X⁻ in which x stands for an integer from 1 to 4, R¹ and R², independently of each other, stand for C₁₋₄-alkyl groups, R³ stands for a C₁₋₂₂-alkyl group or a benzyl group and X— stands for a physiologically acceptable anion. In this general structure, it is preferable for x to stand for the number 3, R¹ and R² each for a methyl group, and R³ either for a methyl group or for a saturated or unsaturated, linear or branched hydrocarbon chain with 8 to 22, especially 10 to 18, carbon atoms.

Halide, especially chloride, bromide and fluoride, sulfate ions and phosphate ions, as well as organic ions such as lactate, citrate, acetate, tartrate, methosulfate and tosylate are examples of physiologically acceptable anions.

Two preferred UV filters with cationic groups are the commercially available compounds cinnamidopropyl-trimethylammonium chloride (Incroquat® UV-283) and dodecyl-dimethylaminobenzamidopropyl-dimethylammonium tosylate (Escalol® HP 610).

Obviously, the teaching according to the invention also includes use of a combination of multiple UV filters. In this embodiment, the combination of at least one water-insoluble UV filter with at least one UV filter having a cationic group is preferred.

The agents according to the invention usually comprise the UV filters (I) in proportions of 0.1-5% by weight, based on the complete agent. Proportions of 0.4-2.5% by weight are preferred.

The agents according to the invention can also contain a 2-pyrrolidinone-5-carboxylic acid and derivatives of it (J). The sodium, potassium, calcium, magnesium or ammonium salts are preferred, with the ammonium ion bearing one to three C₁ to C₄ alkyl groups along with the hydrogen. The sodium salt is quite specially preferred. The proportions used in the agents according to the invention are preferably 0.05 to 10% by weight, based on the complete agent, especially preferably 0.1 to 5 and particularly 1 to 3% by weight.

Finally, the agents according to the invention can also comprise plant extracts (L).

These extracts are usually prepared by extracting the entire plant. In individual cases, though, it may also be preferable to make the extracts exclusively from flowers and/or leaves of the plant.

With respect to the plant extracts that can be used according to the invention, reference is made particularly to the extracts listed beginning on page 44 of the Third Edition of the Guidelines for Declaration of Ingredients of Cosmetic Agents, published by the Industrieverband Körperpflege- und Waschmittel e. V. (IKW), Frankfurt [Association of the Body Care and Detergent Industry, a registered association].

Extracts preferred according to the invention are particularly the extracts of green tea, oak bark, stinging nettle, witch hazel, hops, henna, chamomile, burdock, horsetail, hawthorn, linden oil, almond, aloe vera, pine needles, horse chestnuts, sandalwood, juniper, coconut, mango, apricot, lemon, wheat, kiwi, melons, orange, grapefruit, sage, rosemary, birch, mallow, lady's smock, creeping thyme, yarrow, thyme, melissa, restharrow, coltsfoot, ladyfinger, meristem, ginseng and ginger.

The extracts that are preferred are those from green tea, oak bark, stinging nettle, witch hazel, hops, chamomile, burdock, horsetail, linden oil, almond, aloe Vera, coconut, mango, apricot, lemon, wheat, kiwi, melon, orange, grapefruit, sage, rosemary, birch, lady's smock, creeping thyme, yarrow, restharrow, meristem, ginseng and ginger.

The extracts from green tea, almond, aloe vera, coconut, mango, apricot, lemon, wheat, kiwi and melon are quite specially preferred for use according to the invention.

Water, alcohols and mixtures of them can be used as extractants to prepare the plant extracts named. The preferred alcohols include the lower alcohols such as ethanol and isopropanol, but especially multifunctional alcohols such as ethylene glycol and propylene glycol, either as the sole extractant or mixed with water. Plant extracts based on water/propylene glycol in the ratio of 1:10 to 10:1 have proved particularly suitable.

The plant extracts can be used according to the invention both in the pure form and in a diluted form. To the extent that they are used in a diluted form, the usually comprise about 2-80% by weight of the active substance, with the extractant or extractant mixture used to obtain them as the solvent.

It can further be preferred to use mixtures of multiple, particularly of two, different plant extracts in the agents according to the invention.

It can also prove advantageous for the agents according to the invention to comprise penetrants and/or swelling agents (M). Those include, for instance, urea and urea derivatives, guanidine and its derivatives, arginine and its derivatives, water glass, imidazole and its derivatives, histidine and its derivatives, benzyl alcohol, glycerol, glycol and glycol ethers, propylene glycol and propylene glycol ethers, such as propylene glycol monoethyl ether, carbonates, bicarbonates, diols and triols, and especially 1,2-diols and 1,3-diols such as 1,2-propanediol, 1,2-pentanediol, 1,2-hexanediol, 1,2-dodecanediol, 1,3-propanediol, 1,6-hexanediol, 1,5-pentanediol and 1,4-butanediol.

It can be advantageous in the sense of the invention for additional short-chain carboxylic acids (N) to support the complex of active substances (A). In the sense of the invention, short-chain carboxylic acids and their derivatives are understood to be carboxylic acids which can be saturated or unsaturated and/or linear or branched or cyclic and/or aromatic and/or heterocyclic, and have a molecular weight less than 750. Saturated or unsaturated, linear or branched carboxylic acids with chain lengths of 1 to 16 C atoms in the chain are preferred in the sense of the invention. Those with a chain length of 1 to 12 C atoms in the chain are quite specially preferred.

In the sense of the invention, the short-chain carboxylic acids can have one, two, three or more carboxyl groups. Carboxylic acids with multiple carboxyl groups are preferred in the sense of the invention, especially dicarboxylic and tricarboxylic acids. The carboxyl groups can occur partially or completely as esters, acid anhydrides, lactones, amides, imidic acids, lactams, lactims, dicarboximides, carbohydrazides, hydrazones, hydroxams, hydroximes, amidines, amidoximes, nitriles, phosphonic or phosphate esters. The carboxylic acids used according to the invention can obviously be substituted along the carbon chain or the ring skeleton. The substituents of the carboxylic acids used according to the invention include C₁-C₈-alkyl, C₂-C₈-alkenyl, aryl, aralkyl and aralkenyl, hydroxymethyl, C₂-C₈ hydroxyalkyl, C₂-C₈-hydroxyalkenyl, aminomethyl, C₂-C₈-aminoalkyl, cyano, formyl, oxo, thioxo, hydroxy, mercapto, amino, carboxyl or imino groups. Preferred substituents are C₁-C₈-alkyl, hydroxymethyl, hydroxy, amino, and carboxy groups. Substituents in the − position are specially preferred. Quite specially preferred substituents are hydroxy, alkoxy and amino groups, in which the amino function can optionally be further substituted with alkyl, aryl, aralkyl and/or alkenyl groups. Also, the phosphonic and phosphate esters are likewise preferred carboxylic acid derivatives.

Examples of carboxylic acids according to the invention include formic acid, acetic acid, propionic acid, butyric acid, isobutyric acid, valeric acid, isovaleric acid, pivalic acid, oxalic acid, malonic acid, succinic acid, glutaric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid citraconic acid, mesaconic acid, camphoric acid, benzoic acid, ortho, meta, or para-phthalic acid, naphthoic acid, toluic acid, hydrotropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolcarboxylic acid, 1,2,4,6,7-naphthalene-pentaacetic acid, malonaldehydic acid, 4-hydroxyphthalamidic acid, 1-pyrazolcarboxylic acid, gallic acid or propanetricarboxylic acid, a dicarboxylic acid selected from the group made up of compounds having the general formula (N-I),

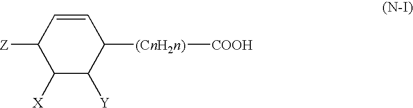

in which Z stands for a linear or branched alkyl or alkenyl group with 4 to 12 carbon atoms, n for a number from 4 to 12, while one of the two groups X and Y stands for a COOH group while the other stands for hydrogen, or for a methyl or ethyl group, dicarboxylic acids of the general formula (N-I)ⱼ which also bear a further 1 to 3 methyl or ethyl substituents on the cyclohexene ring, and dicarboxylic acids that arise formally from the dicarboxylic acids of formula (N-I) by addition of a molecule of water to the double bond in the cyclohexene ring.

Dicarboxylic acids of the formula (N-I) are known in the literature.

The dicarboxylic acids of formula (N-I) can be prepared, for example, by reaction of multiply unsaturated dicarboxylic acids with unsaturated monocarboxylic acids in the manner of a Diels-Alder cyclization. One usually begins with a multiply unsaturated fatty acid as the dicarboxylic acid component. Linoleic acid, which can be obtained from natural fats and oils, is preferred. Acrylic acid, in particular, as well as, for example, methacrylic acid and crotonic acid, is preferred as the monocarboxylic acid component. Diels-Alder reactions usually produce mixtures of isomers, with one component in excess. These isomer mixtures can be used according to the invention as well as the pure compounds.

Aside from the preferred dicarboxylic acids according to formula (N-I), those dicarboxylic acids that differ from the compounds according to formula (N-I) by having 1 to 3 methyl or ethyl substituents on the cyclohexyl ring, or which are formed from those compounds by addition of a molecule of water to the double bond of the cyclohexene ring.

The dicarboxylic acid (mixture) produced by reacting linoleic acid with acrylic acid has proved to be especially effective according to the invention. It is a mixture of 5- and 6-carboxy-4-hexyl-2-cyclohexen-1-octanoic acids. Such compounds are commercially available as Westvaco Diacid® 1550 and Westvaco Diacid® 15695 (Manufacturer: Westvaco).

Aside from the short-chain carboxylic acids themselves that are listed above as used according to the invention, their physiologically acceptable salts can also be used according to the invention. Examples of such salts are the alkali, alkaline earth, and zinc salts, as well as ammonium salts, which are understood in the concept of the present invention to include the mono, di, and trimethyl, ethyl, and hydroxyethyl ammonium salts. In the concept of the invention, though, it can be very specially preferred to use neutralized acids with amino acids that have alkaline reactions, such as arginine, lysine, ornithine and histidine. It can further be preferable, because of formulation, to select the carboxylic acids from the water-soluble representatives, especially the water-soluble salts.

It is further preferred according to the invention to use hydroxycarboxylic acids and, here again, particularly the dihydroxy, trihydroxy, and polyhydroxy carboxylic acids together with the active substance (A). Here it has been found that, aside from the hydroxycarboxylic acids, the hydroxycarboxylic acid esters, and the mixtures of hydroxycarboxylic acids and their esters, as well as polymeric hydroxycarboxylic acids and their esters can be quite particularly preferred. Preferred hydroxycarboxylic acid esters are, for instance, full esters of glycolic acid, lactic acid, malic acid, tartaric acid or citric acid. Other basically suitable hydroxycarboxylic acid esters are esters of β-hydroxypropionic acid, tartronic acid, D-gluconic acid, sugar acids. mucic acid or glucuronic acid. Primary, linear or branched aliphatic alcohols with 8-22 C atoms, such as fatty alcohols or synthetic fatty alcohols, are suitable alcohol components of these esters. The esters of C₁₂-C₁₅ fatty alcohols are especially preferred. Esters of this type are commercially available, for example, under the tradenames Cosmacol® from EniChem, Augusta Industriale. Specially preferred polyhydroxy-polycarboxyacids are polylactic acid and polytartaric acids, and their esters.

The agents according to the invention can also be formulated as hair-coloring agents, for example, oxidative hair colorants or as the so-called color shampoos. Color shampoos usually comprise one or more direct dyes. However, those dyes can also be used in coloring agents for nuancing. Direct dyes are usually nitrophenylenediamines, nitroaminophenols, azo dyes, anthraquinones or indophenols. Preferred direct dyes are known compounds under the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1 and Acid Black 2, as well as 1,2-diamino-2-nitrobenzene, 2-amino-4-nitrophenol, 1,4-bis(β-hydroxyethyl)-amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitro-diphenylamine-2′-carboxylic acid, 6-nitro-1,2,3,4-tetrahydroquinoxaline, 2-hydroxy-1,4-naphthoquinone, picraminic acid and its salts, 2-amino-6-chloro-4-nitrophenol, 4-ethylamino-3-nitrobenzoic acid and 2-chloro-6-ethylamino-1-hydroxy-4-nitrobenzene.

Oxidative colorants or color shampoos preferred according to the invention are distinguished by comprising at least one direct dye selected from nitrophenylenediamines, nitroaminophenols, azo dyes, anthraquinones or indophenols, preferably from the group of dyes known by the following international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1 and Acid Black 2, as well as 1,2-diamino-2-nitrobenzene, 2-amino-4-nitrophenol, 1,4-bis(β-hydroxyethyl)-amino-2-nitrobenzene, 3-nitro-4-(β-hydroxyethyl)aminophenol, 2-(2-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitro-diphenylamine-2′-carboxylic acid, 6-nitro-1,2,3,4-tetrahydroquinoxaline, 2-hydroxy-1,4-naphthoquinone, picraminic acid and its salts, 2-amino-6-chloro-4-nitrophenol, 4-ethylamino-3-nitrobenzoic acid and 2-chloro-6-ethylamino-1-hydroxy-4-nitrobenzene.

The agents according to the invention can also comprise a direct cationic dye. Those that are preferred are
(a) cationic triphenylmethane dyes, such as Basic Blue 7, Basic Blue 26, Basic Violet 2 and Basic Violet 14.
(b) aromatic systems substituted with a quaternary nitrogen group, such as Basic Yellow 57, Basic Red 76, Basic Blue 99, Basic Brown 16 and Basic Brown 17, as well as
(c) direct dyes containing a heterocycle, having at least one quaternary nitrogen atom, such as those named in claims 6 to 11 of EP-2-998 908, to which explicit reference is made here.

The following compounds in particular are preferred direct cationic dyes of group (c):

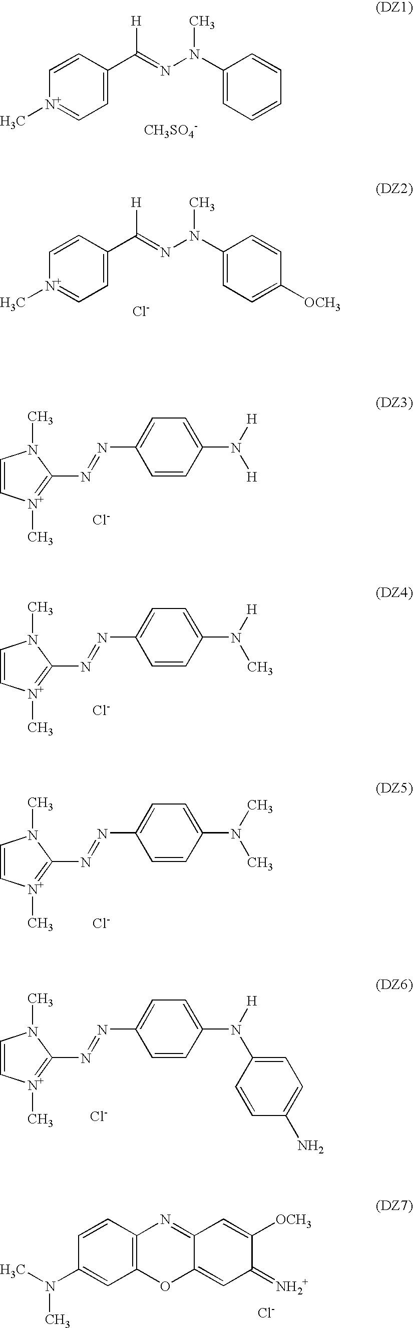

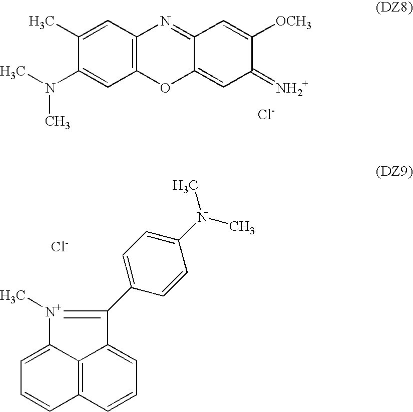

The compounds with formulas (DZ1), (DZ3) and DZ5), which are also known as Basic Yellow 87, Basic Orange 31 and Basic Red 51, are quite specially referred direct cationic dyes of group (c).

The direct cationic dyes marketed under the tradename Arianor® are likewise quite specially preferred direct cationic dyes according to the invention.

The agents according to this embodiment of the invention preferably comprise the direct dyes in proportions of 0.01 to 20% by weight, based on the total coloring agent or color shampoo.

The preparations according to the invention can also comprise naturally occurring colorants, such as Henna red, Henna neutral, Henna black, chamomile flowers, sandalwood, black tea, buckthorn bark, sage, logwood, madder root, catechu, sedre and alkanet root.

The agents according to the invention can be made up as agents for coloring and/or agents for lightening keratinic fibers. In this application, “keratinic or keratin-containing fibers” are understood to be fur, wool, feathers, and especially human hair.

It is also possible according to the invention to prepare a coloring agent (called an “oxidative dye”) or just a bleaching agent (also called a lightening agent). Obviously, products that lighten and color at the same time can also be prepared according to the invention.

Oxidative colorants according to the invention comprise at least one coupler and at least one developer component. Coupler and developer components are also called oxidative dye precursors. In addition, oxidative colorants according to the invention can also comprise direct dyes as nuancers.

The developer components are usually primary aromatic amines with another free or substituted hydroxy or amino group in the ortho or para position, diaminopyridine derivatives, heterocyclic hydrazones, 4-aminopyrazolone derivatives, and 2,4,5,6-tetraminopyrimidine and its derivatives.

Examples of special representatives include p-phenylendiamine, p-toluoylenediamine, 2,4,5,6-tetraminopyrimidine, p-aminophenol, N,N-bis-(2-hydroxyethyl)-p-phenylendiamine, 2-(2,5-diaminophenyl)-ethanol, 2-(2,5-diaminophenoxy)ethanol, 1-phenyl-3-carboxyamido-4-aminopyrazol-5-one: 4-amino-3-methylphenol, 2-aminomethyl-4-aminophenol, 2-hydroxymethyl-4-aminophenol, 2-hydroxy-4,5,6-triaminopyrimidine, 2,4-dihydroxy-5,6-diaminopyrimidine and 2,5,6-triamino-4-hydroxypyridine. As a rule, m-phenylenediamine derivatives, naphthols, resorcinol and resorcinol derivatives, pyrazolone, m-aminophenols and substituted pyridine derivatives are used as coupler components. Particularly suitable coupler substances include α-naphthol, 1,5-, 2,7- and 1,7-dihydroxynaphthalene, 5-amino-2-methylphenol, m-aminophenol, resorcinol, resorcinol monomethyl ether, m-phenylendiamine, 2,4-diaminophenoxyethanol, 2-amino-4-(2-hydroxyethylamino)-anisole (Lehmann's Blue), 1-phenyl-3-methyl-pyrazol-5-one, 2,4-dichloro-3-aminophenol, 1,3-bis-(2,4-diaminophenoxy)-propane, 2-chlororesorcinol, 4-chlororesorcinol, 2-chloro-6-methyl-3-aminophenol, 2-methylresorcinol, 5-methylresorcinol, 3-amino-6-methoxy-2-methylaminopyridine and 3,5-diamino-2,6-dimethoxypyridine.

The present invention is not subject to any limitations about the other dye precursors usable in the colorants according to the invention. The colorants according to the invention can also be used as dye precursors.
oxidative dye precursors of the developer and/or coupler type and
precursors of dyes analogous to the natural ones, such as indole derivatives and indoline derivatives.
   as well as mixtures of members of these groups.

In a first preferred embodiment the colorant also comprises at least one developer component. The developer components are usually primary aromatic amines with another free or substituted hydroxy or amino group in the ortho or para position, diaminopyridine derivatives, heterocyclic hydrazones, 4-aminopyrazolone derivatives, and 2,4,5,6-tetraminopyrimidine and its derivatives.

It can be preferable according to the invention to use a p-phenylendiamine derivative or one of its physiologically acceptable salts as the developer component. Para-phenylenediamine derivatives of the formula (EI) are specially preferred

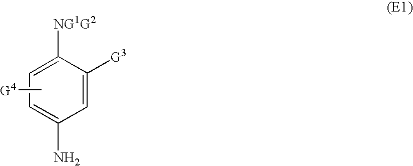

in which
G¹ stands for a hydrogen atom, a C₁ to C₄ alkyl group, a C₁ to C₄ monohydroxyalkyl group, a C₂-C₄ polyhydroxyalkyl group, a (C₁ to C₄)-alkoxy-(C₁ to C₄)-alkyl group, a 4′-aminophenyl group or a C₁ to C₄ alkyl group substituted by a nitrogen-containing group, a phenyl group, or a 4′-aminophenyl group;
G² stands for a hydrogen atom, a C₁ to C₄ alkyl group, a C₁ to C₄ monohydroxyalkyl group, a C₂-C₄ polyhydroxyalkyl group, a (C₁ to C₄)-alkoxy-(C₁ to C₄)-alkyl group, a 4′-aminophenyl group or a C₁ to C₄ alkyl group substituted by a nitrogen-containing group;
G³ stands for a hydrogen atom, a halogen atom such as a chlorine, bromine, iodine or fluorine atom, C₁ to C₄ alkyl group, a C₁ to C₄ monohydroxyalkyl group, a C₂-C₄ polyhydroxyalkyl group, a C₁ to C₄ hydroxyalkyl group, a C₁ to C₄ acetylaminoalkoxy group, a C₁ to C₄ mesylaminoalkoxy group or a C₁ to C₄ carbamoylaminoalkoxy group;
G⁴ stands for a hydrogen atom, a halogen atom, or a C₁ to C₄ alkyl group or
if G³ and G⁴ are in ortho positions with respect to each other, they can jointly form a bridging α,Ω-alkylendioxy group such as an ethylenedioxy group.

The methyl, ethyl, propyl, isopropyl and butyl groups are examples of C₁ to C₄ alkyl groups named as substituents in the compounds according to the invention. Ethyl and methyl are preferred alkyl groups. The methoxy and ethoxy groups are examples of C₁ to C₄ alkoxy groups preferred according to the invention. Other preferred examples of C₁ to C₄ alkoxy groups include hydroxymethyl, 2-hydroxyethyl, 3-hydroxypropyl and the 4-hydroxybutyl group. A 2-hydroxyethyl group is particularly preferred. The 1,2-dihydroxyethyl group is a particularly preferred C₂ to C₄ polyhydroxyalkyl group. Fluoride, chloride or bromide atoms are examples of halogen atoms according to the invention. Chloride atoms are quite specially preferred. The other concepts used are derived according to the invention from the definitions given here. Examples of nitrogen-containing groups of formula (E1) include especially the amino group, C₁-C₄ monoalkylamino groups, C₁ to C₄ dialkylamino groups, C₁ to C₄ trialkylammonium groups, C₁ to C₄ monohydroxyalkylamino groups, imidazolinium and ammonium.

Especially preferred p-phenylenediamines of formula (E1) are selected from p-phenylendiamine, p-toluoylenediamine, 2-chloro-p-phenylendiamine, 2,3-dimethyl-p-phenylendiamine, 2,6-dimethyl-p-phenylendiamine, 2,6-diethyl-p-phenylendiamine, 2,5-dimethyl-p-phenylendiamine, N,N-dimethyl-p-phenylendiamine, N,N-diethyl-p-phenylendiamine, N,N-dipropyl-p-phenylendiamine, 4-amino-3-methyl-(N,N-diethyl)-aniline, N,N-bis-β-hydroxyethyl)-p-phenylendiamine, 4-N,N-bis-(β-hydroxyethyl)amino-2-methylaniline, 4-N,N-bis-(β-hydroxyethyl)amino-2-chloroaniline, 2-(β-hydroxyethyl)-p-phenylendiamine, 2-(α,β-dihydroxyethyl)-p-phenylendiamine, 2-fluoro-p-phenylendiamine, 2-isopropyl-p-phenylendiamine, N-(β-hydroxypropyl)-p-phenylendiamine, 2-hydroxymethyl-p-phenylendiamine, N,N-dimethyl-3-methyl-p-phenylendiamine, N-ethyl, N-β-hydroxyethyl)-p-phenylendiamine, N-(β,γ-dihydroxypropyl)-p-phenylene-diamine, N-(4′-aminophenyl)-p-phenylendiamine, N-phenyl-p-phenylendiamine, 2-(β-hydroxyethoxy)-p-phenylendiamine, 2-(β-acetylaminoethoxy)-p-phenylenediamine, N-(β-methoxyethyl)-p-phenylendiamine and 5,8-diaminobenzo-1,5-dioxane as well as their physiologically acceptable salts.

Quite specially preferred p-phenylendiamine derivatives of the formula (D1) according to the invention are p-phenylendiamine, p-toluoylenediamine, 2-(β-hydroxyethyl)-p-phenylendiamine, 2-(α,β-dihydroxyethyl)-p-phenylendiamine and N,N-bis-(β-hydroxyethyl)-p-phenylendiamine. It can further be preferred according to the invention to use as the developer components compounds containing at least two aromatic nuclei, which are substituted with amino and/or hydroxy groups.

Of the binuclear developer components that can be used in the colorants according to the invention, one can mention in particular the compounds that correspond to the following formula (E2), and their physiologically acceptable salts:

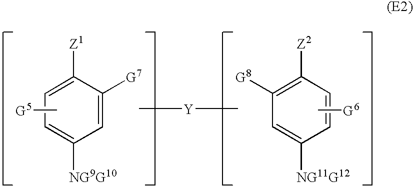

in which:
Z1 and Z2, independently of each other, stand for a hydroxyl or NH₂ group, optionally substituted by a C₁ to C₄ alkyl group, by a C₁ to C₄ hydroxyalkyl group, and/or by a bridge Y or which is optionally part of a bridging ring system,
The bridge Y stands for an alkylene group with 1 to 14 carbon atoms, such as a linear or branched alkylene chain or an alkylene ring, which may be interrupted or terminated by one or more nitrogen-containing groups and/or one or more heteroatoms such as oxygen, sulfur or nitrogen atoms and can perhaps be substituted by one or more hydroxyl or C₁ to C₈ alkoxyl groups, or a direct bond,
G5 and G6 stand, independently of each other, for a hydrogen or halogen atom, a C₁ to C₄ alkyl group, a C₁ to C₄ monohydroxyalkyl group, a C₂ to C₄ polyhydroxyalkyl group, a C₁ to C₄ aminoalkyl group, or a direct bond to the bridge Y,
G7, G8, G9, G10, G11 and G12, independently of each other, stand for a hydrogen atom, a direct bond to the bridge Y, or a C₁ to C₄ alkyl group,
   provided that
the compounds of formula (E2) have only one Y bridge per molecule, and
the compounds of formula (E2) contain at least one amino group that bears at least one hydrogen atom.

The substituents used in formula (E2) are, according to the invention, defined analogously to those presented above.

Preferred binuclear developer components of formula (E2) are, particularly: N,N′-bis-(β-hydroxyethyl)-N,N′-bis-(4-aminophenyl)-1,3-diaminopropan-2-ol,

N,N′-bis-(β-hydroxyethyl)-N,N′-bis-4′-aminophenyl)-ethylenediamine, N,N′-bis-(4-aminophenyl)-tetramethylenediamine, N,N′-bis-(β-hydroxyethyl)-N,N′-bis-(4-aminohenyl)-tetramethylenediamine, N,N′-bis-(4-methylaminophenyl)-tetramethylenediamine, N,N′-diethyl-N,N′-bis-(4′-amino-3′-methylphenyl)-ethylenediamine, bis-(2-hydroxy-5-aminophenyl)-methane, 1,3-bis-(2,5-diaminophenoxy)-propan-2-ol, N,N′-bis-(4′-aminophenyl)-1,4-diazacycloheptane, N,N′-bis(2-hydroxy-5-aminobenzyl)-piperazine, N-(4′-aminophenyl)-p-phenylendiamine and 1,10-bis-(2′,5′-diaminophenyl)-1,4,7,10-tetraoxadecane and their physiologically acceptable salts.

Quite specially preferred binuclear developer components of the formula (E2) are N,N′-bis-(β-hydroxyethyl)-N,N′-bis-(4-aminophenyl)-1,3-diaminopropan-2-ol, bis-(2-hydroxy-5-aminophenyl)-methane, 1,3-bis-(2,5-diaminophenoxy)-propan-2-ol, N,N′-bis-(4′-aminophenyl)-1,4-diazacycloheptane, and 1,10-bis-(2′,5′-diaminophenyl)-1,4,7,10-tetraoxadecane and their physiologically acceptable salts.

It can further be preferred according to the invention to use as developer components a p-aminophenol derivative or one of its physiologically acceptable salts. Para-aminophenol derivatives of formula (E3) are specially preferred

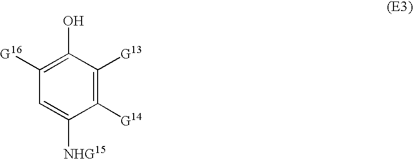

in which
G¹³ stands for a hydrogen atom, a halogen atom, a C₁ to C₄ alkyl group, a C₁ to C₄ monohydroxyalkyl group, a C₂ to C₄ polyhydroxyalkyl group, a (C₁ to C₄)-alkoxy-(C₁ to C₄)-alkyl group, a C: to C₄ aminoalkyl group, a hydroxy-(C₁ to C₄)-alkylamino group, a C₁ to C₄ hydroxyalkoxy group, a C₁ to C₄ hydroxyalkyl-(C₁ to C₄)-aminoalkyl group or a (di-(C₁ to C₄)alkylamino)-(C₁ to C₄) alkyl group, and
G¹⁴ stands for a hydrogen or halogen atom, a C₁ to C₄ alkyl group, a C₁ to C₄ monohydroxyalkyl group, a C₂ to C₄ polyhydroxyalkyl group, a (C₁ to C₄)-alkoxy-(C₁ to C₄)-alkyl group, a C₁ to C₄ aminoalkyl group or a C₁ to C₄ cyanoalkyl group,
G¹⁵ stands for hydrogen, a C₁ to C₄ alkyl group, a C₁ to C₄ monohydroxyalkyl group, a C₂ to C₄ polyhydroxyalkyl group, a phenyl group, or a benzyl group, and
G¹⁶ stands for hydrogen or a halogen atom.

The substituents used in formula (E3) are, according to the invention, defined analogous to those presented above.

Preferred p-aminophenols of formula (E3) are p-aminophenol, N-methyl-p-aminophenol, 4-amino-3-methylphenol, 4-amino-3-fluorophenol, 2-hydroxymethylamino-4-aminophenol, 4-amino-3-hydroxymethylphenol, 4-amino-2(-hydroxyethoxy)-phenol, 4-amino-2-methylphenol, 4-amino-2-hydroxymethylhenol, 4-amino-2-methoxymethylphenol, 4-amino-2-aminomethylphenol, 4-amino-2-(β-hydroxyethylaminomethyl)-phenol, 4-amino-2-(α,β-dihydroxyethyl)-phenol, 4-amino-2-fluorophenol, 4-amino-2-chlorophenol, 4-amino-2,6-dichlorophenol, 4-amino-2-(diethylaminomethyl)-phenol and their physiologically acceptable salts.

Quite specially preferred compounds of formula (E3) are p-aminophenol, 4-amino-3-methylphenol, 4-amino-2-aminomethylphenol, 4-amino-2-(α,β-dihydroxyethyl)-phenol, and 4-amino-2-(diethylaminomethyl)-phenol.

The developer components can further be selected from o-aminophenol and its derivatives, such as 2-amino-4-methylphenol, 2-amino-5-methylphenol or 2-amino-4-chlorophenol.

The developer components can further be selected from heterocyclic developer components such as pyridine, pyrimidine, pyrazole, pyrazole-pyrimidine derivatives and their physiologically acceptable salts.

Preferred pyridine derivatives are in particular the compounds 2,5-diaminopyridine, 2-(4′-methoxyphenyl)amino-3-aminopyridine, 2,3-diamino-6-methoxypyridine, 2-(β-methoxyethyl)amino-3-amino-6-methoxypyridine) and 3,4-diaminopyridine.

Preferred pyrimidine derivatives are in particular the compounds 2,4,5,6-tetraminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine and 2,5,6-triaminopyrimidine.

Preferred pyrazole derivatives are in particular 4,5-diamino-1-methylpyrazaole, 4,5-diamino-1-(β-hydroxyethyl)-pyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert.-butyl-1-methylpyrazole, 4,5-diamino-1-tert.-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(-aminoethyl)amino-1,3-dimethylpyrazole, 3,4,5-triaminopyrazole, 1-methyl-3,4,5-triaminopyrazole, 3,5-diamino-1-methylaminopyrazole and 3,5-diamino-4-(β-hydroxyethyl)amino-1-methylpyrazole.

Particularly preferred pyrazole-pyrimidine derivatives are the derivatives of pyrazol-[1,5-a]-pyrimidine of the following formula (E4) and its tautomeric forms, if there is a tautomeric equilibrium:

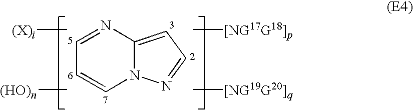

in which:
G17, G18, G19 and G20, independently of each other, stand for a hydrogen atom, a C₁ to C₄ alkyl group, an aryl group, a C₁ to C₄ hydroxyalkyl group, a C₂ to C₄ polyhydroxyalkyl group, a (C₁ to C₄)-alkoxy-(C₁ to C₄)-alkyl group, a C₁ to C₄ aminoalkyl group that can optionally be protected by an acetyl-ureido or a sulfonyl group, a (C₁ to C₄)-alkylamino-(C₁ to C₄)-alkyl group, a di-[(C₁ to C₄)-alkyl]-(C₁ to C₄)aminoalkyl group in which the dialkyl groups can optionally form a carbon cycle or a heterocycle with 5 or 6 chain elements, a C: to C₄ hydroxyalkyl or a di-(C₁ to C₄)-[hydroxyalkyl]-(C₁ to C₄)-aminoalkyl group,
the X groups, independently of each other, stand for a hydrogen atom, a C₁ to C₄ alkyl group, an aryl group, a C₁ to C₄ hydroxyalkyl group, a C₂ to C₄ polyhydroxyalkyl group, a C₁ to C₄ aminoalkyl group, a (C₁ to C₄)-alkylamino-(C₁ to C₄)-alkyl group, a di-[(C₁ to C₄)-alkyl]-(C₁ to C₄)aminoalkyl group in which the dialkyl groups can optionally form a carbon cycle or a heterocycle with 5 or 6 chain elements, a C₁ to C₄ hydroxyalkyl group or a di-(C₁ to C₄)-[hydroxyalkyl]-(C₁ to C₄)-aminoalkyl group, a halogen atom, a carboxylic acid group or a sulfonic acid group,
i has the value 0, 1, 2 or 3,
p has the value 0 or 1
q has the value 0 or 1 and
n has the value 0 or 1,
   provided that
the sum of p+1 is not equal to 0,
if p+2 is equal to 2, n has the value 0 and the groups NG17G18 and NG19G20 occupy the positions (2,3), (5,6), 6,7) or (3,7),
if p+q is equal to 1, n has the value 1 and the groups NG17G18 (or NG19G20) and the group OH occupy the positions (2,3), (5,6), 6,7), 3,5) or (3,7).

The substituents used in formula (E4) are, according to the invention, defined analogously to those presented above.

If the pyrazol-[1,5-a]-pyrimidine of the formula (E4) above contains a hydroxy group at one of positions 2, 5 or 7 of the ring system, there is a tautomeric equilibrium, shown, for example, in the following diagram:

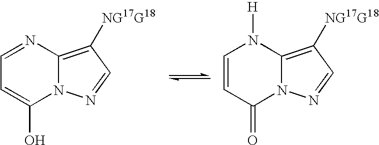

Among the pyrazol-[1,5-a]-pyrimidines of formula (E4) above, one can name in particular:
pyrazol-[1,5-a]-pyrimidin-3,6-diamine;
2,5-dimethyl-pyrazol-[1,5-a]-pyrimidin-3,7-diamine;
pyrazol-[1,5-a]-pyrimidin-3,5-diamine;
2,7-dimethyl-pyrazol-[1,5-a]-pyrimidin-3,5-diamine;
3-aminopyrazol-[1,5-a]-pyrimidin-7-ol;
3-aminopyrazol-[1,5-a]-pyrimidin-5-ol;
2-(3-aminopyrazol-[1,5-a]-pyrimidin-7-ylamino)-ethanol;
2-(7-aminopyrazol-[1,5-a]-pyrimidin-3-ylamino)-ethanol;
2-[(3-aminopyrazol-[1,5-a]-pyrimidin-7-yl)-(2-hydroxyethyl)amino]-ethanol;
2-[(7-aminopyrazol-[1,5-a]pyrimidin-3-yl)-(2-hydroxyethyl)amino]-ethanol;
5,6-dimethylpyrazol-[1,5-a]-pyrimidin-3,7-diamine;
2,6-dimethylpyrazol-[1,5-a]-pyrimidin-3,7-diamine;
3-amino-7-dimethylamino-2,5-dimethylpyrazol-[1,5-a]-pyrimidine;
   as well as their physiologically acceptable salts and their tautomeric form, if there is a tautomeric equilibrium.

The pyrazol-[1,5-a]-pyrimidines of formula (E4) above can be prepared by cyclizing, as described in the literature, starting with an aminopyrazole or with hydrazine.

Oxidative colorants specially preferred according to the invention are characterized by the developer component being selected from 3-methyl-1,2-diaminobenzene, 1-(2′-hydroxyethyl)-2,5-diaminobenzene, 2-(2,5-diaminophenoxy)-ethanol, N,N-bis-(2′-hydroxyethyl)-1,4-diaminobenzene, 3-methyl-4-aminophenol and 2-methyl-4-aminophenol, p-phenylendiamine, 2-(β-hydroxyethyl)-p-phenylendiamine, N,N-bis-(β-hydroxyethyl)-p-phenylendiamine, N,N′-bis-(β-hydroxyethyl)-N,N′-bis(4′-aminophenyl)-1,3-diaminopropan-2-ol, bis-(2-hydroxy-5-aminophenyl)-methane, N,N′-bis-(4′-aminophenyl)-1,4-diazacycloheptane, 1,10-bis-(2′,5′-diaminophenyl)-1,4,7,10-tetraoxadecane, p-aminophenol, 4-amino-3-fluorophenol, 4-amino-2-aminomethylphenol, 4-amino-2-((diethylamino)methyl)phenol, o-aminophenol, 2-amino-4-methylphenol, 2-amino-5-methylphenol, 2-amino-4-chlorophenol, 2,4,5,6-tetraminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine, 2,5,6-triaminopyrimidine, 1-(2′-hydroxy-5′-aminobenzyl)-imidazolidin-3-one, 4,5-diamino-1-(2′-hydroxyethyl)pyrazole and N-(4-amino-3-methylphenyl)-N-[3-(1H-imidazol-1-yl)propyl]amine trihydrochloride.

The colorants according to the invention contain at least one coupler component.

As a rule, m-phenylenediamine derivatives, naphthols, resorcinol and resorcinol derivatives, pyrazolone and m-aminophenol derivatives are used as coupler components. Examples of particularly suitable coupler substances include 1-naphthol, 1,5-, 2,7-, and 1,7-dihydroxynaphthalene, 5-amino-2-methylphenol, m-aminophenol, resorcinol, resorcinol monomethyl ether, m-phenylenediamine, 1-phenyl-3-methyl-pyrazolone-5,2,4-dichloro-3-aminophenol, 1,3-bis-(2′,4′-diaminophenoxy)-propane, 2-chlororesorcinol, 4-chlororesorcinol, 2-chloro-6-methyl-3-aminophenol, 2-amino-3-hydroxypyridine, 3-methylresorcinol, 5-methylresorcinol and 2-methyl-6-chloro-5-aminohenol.

Coupler components preferred according to the invention are
m-aminophenol and its derivatives, such as 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-(diethylamino)phenol, N-cyclopentyl-3-aminophenol, 1,2-dihydroxy-5-(methylamino)benzene, 3-ethylamino-4-methylphenol and 2,4-dichloro-3-aminophenol,
o-aminophenol and its derivatives,
m-diaminobenzene and its derivatives such as 2,4-diaminophenoxyethanol, 1,3-bis-(2′,4′-diaminophenoxy)propane, 1-methoxy-2-amino-4-(2′-hydroxyethylamino)benzene, 1,3-bis-(2′,4,′-diaminophenyl)-propane, 2,6-bis-(2′-hydroxyethylamino)-1-methylbenzene and 1-amino-3-bis-(2′-hydroxyethyl)-aminobenzene,
o-diaminobenzene and its derivatives such as 3,4-diaminobenzoic acid and 2,3-diamino-1-methyl-benzene,
di- or tri-hydroxybenzene derivatives such as resorcinol, resorcinol monomethyl ether, 2-methylresorcinol, 4-methylresorcinol, 2,5-dimethylresorcinol, 2-chlororesorcinol, 4-chlororesorcinol, pyrogallol and 1,2,4-trihydroxybenzene,
pyridine derivatives such as 2,6-dihydroxypyridine, 2-amino-3-hydroxypyridine, 2-amino-5-chloro-3-hydroxypyridine, 2-amino-2-methylamino-6-methoxypyridine, 2,6-dihydroxy-3,4-dimethylpyridine, 2,6-dihydroxy-4-methylpyridine, 2,6-diaminoyridine, 2,3-diamino-6-methoxypyridine and 3,5-diamino-2,6-dimethoxypyridine,
naphthalene derivatives such as 1-naphthol, 2-methyl-1-naphthol, 2-hydroxymethyl-1-naphthol, 2-hydroxyethyl-1-naphthol, 1,5-dihydroxynaphthalene, 1,6-dihydroxynaphthalene, 1,7-dihydroxynaphthalene, 1,8-dihydroxynaphthalene, 2,7-dihydroxynaphthalene and 2,3-dihydroxynaphthalene,
morpholine derivatives such as 6-hydroxybenzomorpholine and 6-amino-benzomorpholine,
quinoxaline derivatives such as 6-methyl-1,2,3,4-tetrahydroquinoxaline,
pyrazol derivatives such as 1-phenyl-3-methylpyrazol-5-one,
indole derivatives such as 4-hydroxyindole, 6-hydroxyindole and 7-hydroxyindole,
pyrimidine derivatives such as 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine, or
methylenedioxybenzene derivatives such as 1-hydroxy-3,4-methylenedioxybenzene, 1-amino-3,4-methylenedioxybenzene and 1-(2′-hydroxyethyl)amino-3,4-methylenedioxybenzene.

Coupler components preferred according to the invention are 1-naphthol, 1,5-, 2,7- and 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 2,5-dimethylresorcinol and 2,6-dihydroxy-3,4-dimethylpyridine.

Those indole and indoline derivatives that have at least one hydroxy or amino group, preferably as a substituent on the six-membered ring, are preferred as precursors of colorants analogous to the natural ones. These groups can bear other substituents, e.g., in the form of etherification or esterification of the hydroxy group or alkylation of the amino group. In a second preferred embodiment, the colorants comprise at least one indole or indoline derivative.

Those derivatives of 5,6-dihydroxyindoline of formula (IIIa)

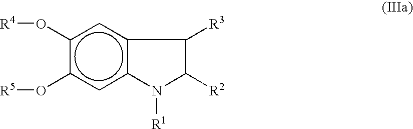

in which, independently of each other,
R¹ stands for hydrogen, a C₁-C₄ alkyl group or a C₁ to C₄ hydroxyalkyl group,
R² stands for hydrogen or a —COOH group, in which the COOH group can also occur as a salt with a physiologically acceptable cation,
R³ stands for hydrogen or for a C₁-C₄ alkyl group,
R⁴ stands for hydrogen, a C₁-C₄ alkyl group or a group —CO—R⁶, in which R⁶ stands for a C₁-C₄ alkyl group, and
R⁵ stands for one of the groups named under R⁴,
   and the physiologically acceptable salts of these compounds with an organic or inorganic acid are particularly well suited as precursors of hair colorants analogous to the natural ones.

Specially preferred derivatives of indoline are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid, as wells as 6-hydroxyindoline, 6-aminoindoline and 4-aminoindoline.

In this group, the following are specially emphasized: N-methyl-5,6-dihydroxyindoline, N-ethyl-5,6-dihydroxyindoline, N-propyl-5,6-dihydroxyindoline, N-butyl-5,6-dihydroxyindoline and particularly 5,6-dihydroxyindoline.

Further derivatives of 5,6-dihydroxyindole of formula (IIIb)

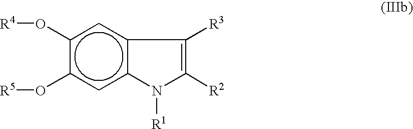

in which, independently of each other,
R¹ stands for hydrogen, a C₁-C₄ alkyl group or a C₁ to C₄ hydroxyalkyl group,
R² stands for hydrogen or a —COOH group, in which the COOH group can also occur as a salt with a physiologically acceptable cation,
R³ stands for hydrogen or for a C₁-C₄ alkyl group,
R⁴ stands for hydrogen, a C₁-C₄ alkyl group or a group —CO—R⁶, in which R⁶ stands for a C₁-C₄ alkyl group, and
R⁵ stands for one of the groups named under R⁴,
and the physiologically acceptable salts of these compounds with an organic or inorganic acid are outstandingly suited as precursors of hair colorants analogous to the natural ones.

Specially preferred derivatives of indole are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid. 6-hydroxyindole, 6-aminoindole and 4-aminoindole.

Within this group, the following must be emphasized: N-methyl-5,6-dihydroxyindole, N-ethyl-5,6-dihydroxyindole, N-propyl-5,6-dihydroxyindole, N-butyl-5,6-dihydroxyindole and particularly 5,6-dihydroxyindole.

Within the process according to the invention, the indoline and indole derivatives can be used both as the free bases and in the form of their physiologically acceptable salts with inorganic or organic acids, such as the hydrochloride, sulfate, and hydrobromide. In these, the indole or indoline derivatives are usually comprised in proportions of 0.05-10% by weight, preferably 0.2-5% by weight.

In a further embodiment it can be preferred according to the invention to use the indoline or indole derivative in hair-coloring agents in combination with at least one amino acid or an oligopeptide. The amino acid is advantageously an α-amino acid. Quite specially preferred α-amino acids are arginine, ornithine, lysine, serine and histidine, particularly arginine.

Oxidative colorants preferred according to the invention are characterized by the coupler component being selected from m-phenylenediamine derivatives, naphthols, resorcinol and resorcinol derivatives, pyrazolones, m-aminophenols and substituted pyridine derivatives, with preferred substances being resorcinol, 3-amino-2-methylamino-6-methoxypyridine, 3-amino-6-methylphenol, 3-amino-2-hydroxypyridine, 1,3-bis-(2,4-diaminophenoxy)propane, 2,7-dihydroxynaphthalene, 2-methylresorcinol, 5-methylresorcinol, 2,5-dimethylresorcinol and 4-chlororesorcinol, comprising 1-naphthol, 1,5-, 2,7- and 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcinol, 2,6-dihydroxy-3,4-dimethylpyridine, 2-({3-[(2-hydroxyethyl)amino-4-methoxy-5-methylphenyl}amino)ethanol, 2-({3-[(2-hydroxyethyl)amino-2-methoxy-5-methylphenyl}amino)ethanol, 3-amino-4-(2-methoxyethoxy)-5-methyl-phenylamine and/or 2-[(3-morpholin-4-ylphenyl)amino]ethanol dihydrochloride.

It is preferable to use the coupler and developer components in a certain mutual ratio. Here, oxidative colorants are preferred which comprise the coupler component(s) in a proportion of 0.01 to 20% by weight, preferably 0.5 to 5% by weight, and the developer component(s) in a proportion of 0.01 to 20% by weight, preferably 0.5 to 5% by weight, both based on the complete oxidative colorant.

It is not necessary that the oxidative colorant precursors or the direct dyes be single compounds. Rather, the hair colorants according to the invention can comprise still other components in subordinate proportions, due to the manufacturing processes for the individual dyes, as long as they do not have a bad effect on the resultant color or do not have to be excluded for other reasons such as toxicology.

With respect to dyes usable as hair coloring and toning agents, explicit reference is made to the monograph by Ch. Zviak, The Science of Hair Care, Chapter 7 (pages 248-250); direct dyes) and Chapter 8, pages 264-267; oxidative colorant precursors), which appears as Volume 7 in the series “Dermatology” (Eds.: Ch. Culnan and H. Maibach), Verlag Marcel Dekker Inc., New York, Basel, 1986, and the “European Inventory of Cosmetic Raw Materials”, issued by the European Community, available in diskette form from the Bundesverband Deutscher Industrie- und Handelsunternehmen für Arzneimittel, Reformwaren und Körperpflegemiottel e. V. [Federal Association of German Industrial and Commercial Companies for Medications, Health Foods and Body Care Agents [registered association], Mannheim.

Thus in another embodiment the agent according to the invention comprises a combination of components
A compounds that comprise a carbonyl group that is reactive with components
B compounds selected from (a) CH-acidic compounds, (b) compounds with primary or secondary amino groups or hydroxy groups, selected from primary or secondary aromatic amines, nitrogen-containing heterocyclic compounds and aromatic hydroxy compounds, (c) amino acids and (d) oligopeptides built up of 2 to 9 amino acids.

Compounds according to the invention having a reactive carbonyl group (also called reactive carbonyl compounds in the following, or component A) have at least one carbonyl group as the reactive group that reacts with compounds of component B, forming a chemical bond that links the two components together. Further, according to the invention, component A also includes those compounds in which the reactive carbonyl group is derivatized or masked, so that the reactivity of the carbon atom of the derivatized or masked carbonyl group for component B always exists. These derivatives are preferably condensation compounds of reactive carbonyl compounds with
a) amines and their derivatives, with formation of imines or oximes as the condensation compound
b) alcohols, forming acetals or ketals as the condensation compound
c) water, forming hydrates as the addition compound.

The A components are preferably selected from the group made up of acetophenone, propiophenone, 2-hydroxyacetophenone, 3-hydroxyacetophenone, 4-hydroxyacetophenone, 2-hydroxypropiophenone, 3-hydroxypropiophenhone, 4-hydroxypropiophenone, 2-hydroxybutyrophenone, 3-hydroxybutyrophenone, 4-hydroxybutyrophenone, 2,4-dihydroxyacetophenone, 2,5-dihydroxyacetophenone, 2,6-dihydroxyacetophenone, 2,3,4-trihydroxy-acetophenone, 3,4,5-trihydroxyacetophenone, 2,4,6-trihydroxyacetophenone, 2,4,6-trimethoxyacetophenone, 3,4,5-trimethoxy-acetophenone, 3,4,5-trimethoxy-acetophenone diethyl ketal, 4-hydroxy-3-methoxy-acetophenone, 3,5-dimethoxy-acetophenone, 4-aminoacetophenone, 4-dimethylamino-acetophenone, 4-morpholinoacetophenone, 4-piperidino-acetophenone, 4-imidazolinoacetophenone, 2-hydroxy-5-bromo-acetophenone, 4-hydroxy-3-nitro-acetophenone, acetophenone-2-carboxylic acid, acetophenone-4-carboxylic acid, benzophenone, 4-hydroxybenzophenone, 2-aminobenzo-phenone, 4,4′-dihydroxy-benzophenone, 2,4-dihydroxybenzophenone, 2,4-dihydroxy-benzophenone, 2,4,4′-trihydroxy-benzophenone, 2,3,4-trihydroxy-benzophenone, 2-hydroxy-1-acetonaphthone, 1-hydroxy-2-acetonaphthone, chromone, chromone-2-carboxylic acid, flavone, 3-hydroxyflavone, 3,5,7-trihydroxyflavone, 4′,5,7-trihydroxyflavone, 5,6,7-trihydroxyflavone, Quercetin, 1-indanone, 9-fluorenone, 2-hydroxyfluorenone, anthrone, 1,8-dihydroxyanthrone, vanillin, coniferyl aldehyde, 2-methoxy-benzaldehyde, 3-methoxybenzaldehyde, 4-methoxy-benzaldehyde, 2-ethoxy-benzaldehyde, 3-ethoxy-benzaldehyde, 4-ethoxy-benzaldehyde, 4-hydroxy-2,3-dimethoxy-benzaldehyde, 4-hydroxy-2,5-dimethoxy-benzaldehyde, 4-hydroxy-2,6-dimethoxy-benzaldehyde, 4-hydroxy-2-methyl-benzaldehyde, 4-hydroxy-3-methyl-benzaldehyde, 4-hydroxy-2,3-dimethyl-benzaldehyde, 4-hydroxy-2,5-dimethyl-benzaldehyde, 4-hydroxy-2,6-dimethyl-benzaldehyde, 4-hydroxy-3,5-dimethoxy-benzaldehyde, 4-hydroxy-3,5-dimethyl-benzaldehyde, 3,5-diethoxy-4-hydroxy-benzaldehyde, 2,6-diethoxy-4-hydroxy-benzaldehyde, 3-hydroxy-4-methoxy-benzaldehyde, 2-hydroxy-4-methoxy-benzaldehyde, 2-ethoxy-4-hydroxy-benzaldehyde, 3-ethoxy-4-hydroxy-benzaldehyde, 4-ethoxy-2-hydroxy-benzaldehyde, 4-ethoxy-3-hydroxy-benzaldehyde, 2,3-dimethoxy-benzaldehyde, 2,4-dimethoxy-benzaldehyde, 2,5-dimethoxy-benzaldehyde, 2,6-dimethoxy-benzaldehyde, 3,4-dimethoxy-benzaldehyde, 3,5-dimethoxy-benzaldehyde, 2,3,4-trimethoxybenzaldehyde, 2,3,5-trimethoxybenzaldehyde, 2,3,6-trimethoxybenzaldehyde, 2,4,6-trimethoxybenzaldehyde, 2-hydroxy-benzaldehyde, 3-hydroxybenzaldehyde, 4-hydroxy-benzaldehyde, 2,3-dihydroxy-benzaldehyde, 2,4-dihydroxy-benzaldehyde, 2,5-dihydroxy-benzaldehyde, 2,6-dihydroxy-benzaldehyde, 3,4-dihydroxy-benzaldehyde, 3,5-dihydroxy-benzaldehyde, 2,3,4-trihydroxybenzaldehyde, 2,3,5-trihydroxybenzaldehyde, 2,3,6-trihydroxybenzaldehyde, 2,4,6-trihydroxybenzaldehyde, 2,4,5-trihydroxybenzaldehyde, 2,5,6-trihydroxybenzaldehyde, 4-hydroxy-2-methoxy-benzaldehyde, 4-dimethylamino-benzaldehyde, 4-diethylamino-benzaldehyde, 4-diethylamino-2-hydroxy-benzaldehyde, 4-pyrrolidino-benzaldehyde, 4-morpholino-benzaldehyde, 2-morpholino-benzaldehyde, 4-piperidino-benzaldehyde, 2-methoxy-1-naphthaldehyde, 4-methoxy-1-naphthaldehyde, 2-hydroxy-1-naphthaldehyde, 2,4-dihydroxy-1-naphthaldehyde, 4-hydroxy-3-methoxy-1-naphthaldehyde, 2-hydroxymethoxy-1-naphthaldehyde, 3-hydroxy-4-methoxy-1-naphthaldehyde, 2,4-dimethoxy-1-naphthaldehyde, 3,4-dimethoxy-1-naphthaldehyde, 4-hydroxy-1-naphthaldehyde, 4-dimethylamino-1-naphthaldehyde, 2-methoxy-cinnamaldehyde, 4-methoxy-cinnamaldehyde, 4-hydroxy-3-methoxy-cinnamaldehyde, 3,5-dimethoxy-4-hydroxy-cinnamaldehyde, 4-dimethylamino-cinnamaldehyde, 2-dimethylamino-benzaldehyde, 2-chloro-4-dimethylamino-benzaldehyde, 4-dimethylamino-2-methyl-benzaldehyde, 4-diethylamino-cinnamaldehyde, 4-dibutylamino-benzaldehyde, 4-diphenylamino-benzaldehyde, 4-dimethylamino-2-methoxy-benzaldehyde, 4-(1-imidazolyl)-benzaldehyde, piperonal, 2,3,6,7-tetrahydro-1H,5H-benzo-[i]-quinolizin-9-carboxaldehyde, 2,3,6,7-tetrahydro-8-hydroxy-1H,5H-benzo-[i]-quinolizin-9-carboxaldehyde, N-ethylcarbazol-3-aldehyde, 2-formylmethylene-1,3,3-trimethylindoline (Fischer's aldehyde or Tribasic aldehyde), 2-indolaldehyde, 3-indolaldehyde, 1-methylindol-3-aldehyde, 2-methylindol-3-aldehyde, 1-acetylindol-3-aldehyde, 3-acetylindole, 1-methyl-3-acetylindole, 2-(1′,3′,3′-trimethyl-2-indolinylidene)-acetaldehyde, 1-methylpyrrol-2-aldehyde, 1-methyl-2-acetylpyrrole, 4-pyridine aldehyde, 2-pyridine aldehyde, 3-pyridine aldehyde, 4-acetylpyridine, 2-acetylpyridine, 3-acetylpyridine, pyridoxal, quinoline-3-aldehyde, quinoline-4-aldehyde, antipyrin-4-aldehyde, furfural, 5-nitrofurfural, 2-thenoyl-trifluoroacetone, chromone-3-aldehyde, 3-(5′-nitro-2′furyl)acrolein, 3-(2′-furyl)-acrolein and imidazol-2-aldehyde, 1,3-diacetylbenzene, 1,4-diacetylbenzene, 1,3,5-triacetylbenzene, 2-benzoyl-acetophenone, 2-(4′-methoxybenzoyl)-acetophenone, 2-(2′-furyl)-acetophenone, 2-(2′-pyridoyl)-acetophenone, and 2-(3′-pyridoyl)-acetophenone, benzylideneacetone, 4-hydroxy-benzylideneacetone, 2-hydroxy-benzylideneacetone, 4-methoxy-benzylideneacetone, 4-hydroxy-3-methoxy-benzylideneacetone, 4-dimethylamino-benzylideneacetone, 3,4-methylendioxy-benzylideneacetone, 4-pyrrolidono-benzylideneacetone, 4-piperidino-benzylideneacetone, 4-morpholino-benzylideneacetone, 4-diethylamino-benzylideneacetone, 3-benzylidene-2,4-pentanedione, 3,(4′-hydroxy-benzylidene)-2,4-pentanedione, 3-(4′-dimethylaminobenzylidene)-2,4-pentanedione, 2-benzylidene-cyclohexanone, 2-(4′-hydroxy-benzylidene)-cyclohexanone, 2-(4′-dimethylaminobenzylidene)cyclohexanone, 2-benzylidene-1,3-cyclohexanedione, 2-(4′-hydroxy-benzylidene)-1,3-cyclohexanedione, 3-(4′-dimethylamino-benzylidene)-1,3-cyclohexanedione, 2-benzylidene-5,5-dimethyl-1,3-cyclohexanedione, 2-(4′-hydroxy-benzylidene)-5,5-dimethyl-1,3-cyclohexanedione, 2-(4′-hydroxy-3-methoxy-benzylidene)-5,5-dimethyl-1,3-cyclohexanedione, 2-(4′-dimethylaminobenzylidene)-5,5-dimethyl-1,3-cyclohexanedione, 2-benzylidene-cyclopentanone, 2′-(4-hydroxy-benzylidene)-cyclopentanone, 2-(4′-dimethylamino-benzylidene)-cyclopentanone, 5-(4-dimethylaminophenyl)penta-2,4-dienal, 5-(4-diethylaminophenyl)penta-2,4-dienal, 5-(4-dimethoxyphenyl)penta-2,4-dienal, 5-(3,4-dimethoxyphenyl)penta-2,4-dienal, 5-(2,4-dimethoxyphenyl)penta-2,4-dienal, 5-(4-piperidinophenyl)penta-2,4-dienal, 5-(4-morpholinopenyl)penta-2,4-dienal, 5-(4-pyrrolidinophenyl)penta-2,4-dienal, 6-(4-dimethylaminophenyl)hexa-3,5-dien-2-one, 6-(4-diethyl-aminophenyl)-hexa-3,5-dien-2-one, 6-(4-methoxyphenyl)hexa-3,5-dien-2-one, 6-(3,4-dimethoxyphenyl)-hexa-3,5-dien-2-one, 6-(2,4-dimethoxyphenyl)-hexa-3,5-dien-2-one, 6-(4-piperidinophenyl)-hexa-3,5-dien-2-one, 6-(4-morpholinophenyl)-hexa-3,5-dien-2-one, 6-(4-pyrrolidonophenyl)-hexa-3,5-dien-2-one, 5-(4-dimethylamino-1-naphthyl)penta-3,5-dienal, 2-nitro-benzaldehyde, 3-nitro-benzaldehyde, 4-nitro-benzaldehyde, 4-methyl-3-nitro-benzaldehyde, 3-hydroxy-4-nitro-benzaldehyde, 4-hydroxy-3-nitro-benzaldehyde, 5-hydroxy-2-nitro-benzaldehyde, 2-hydroxy-5-nitro-benzaldehyde, 2-hydroxy-3-nitro-benzaldehyde, 2-fluoro-3-nitro-benzaldehyde, 3-methoxy-2-nitro-benzaldehyde, 4-chloro-3-nitro-benzaldehyde, 2-chloro-6-nitro-benzaldehyde, 5-chloro-2-nitro-benzaldehyde, 4-chloro-2-nitro-benzaldehyde, 2,4-dinitro-benzaldehyde, 2,6-dinitro-benzaldehyde, 2-hydroxy-3-methoxy-5-nitro-benzaldehyde, 4,5-dimethoxy-2-nitro-benzaldehyde, 6-nitropiperonal, 2-nitropiperonal, 5-nitrovanillin, 2,5-dinitrosalicylaldehyde, 5-bromo-3-nitrosalicylaldehyde, 3-nitro-4-formyl-benzenesulfonic acid, 4-nitro-1-naphthaldehyde, 2-nitrocinnamaldehyde, 3-nitrocinnamaldehyde, 4-nitrocinnamaldehyde, 9-methyl-3-carbazolaldehyde, 9-ethyl-3-carbazolaldehyde, 3-acetylcarbazole, 3,6-diacetyl-9-ethylcarbazole, 3-acetyl-9-methylcarbazole, 1,4-dimethyl-3-carbazolaldehyde, 1,4,9-trimethyl-3-carbazolaldehyde, 4-formyl-1-methylpyridinium, 2-formyl-1-methylpyridinium, 4-formyl-1-ethylpyridinium, 2-formyl-1-ethylpyridinium, 4-formyl-1-benzylpyridinium, 2-formyl-1-benzylpyridinium, 4-formyl-1,2-dimethylpyridinium, 4-formyl-1,3-dimethylpyridinium, 4-formyl-1-methylquinolinium, 2-formyl-1-methyl-quinolinium, 4-acetyl-1-methylpyridinium, 2-acetyl-1-methylpyridinium, 4-acetyl-1-methyl-quinolinium, 5-formyl-1-methyl-quinolinium, 6-formyl-1-methyl-quinolinium, 7-formyl-1-methyl-quinolinium, 8-formyl-1-methyl-quinolinium, 5-formyl-1-ethyl-quinolinium, 6-formyl-1-ethyl-quinolinium, 7-formyl-1 ethyl-quinolinium, 8-formyl-1-ethyl-quinolinium, 5-formyl-1-benzylquinolinium, 6-formyl-1-benzylquinolinium, 7-formyl-1-benzylquinolinium, 8-formyl-1-benzylquinolinium, 5-formyl-1-allyl-quinolinium, 6-formyl-1-allyl-quinolinium, 7-formyl-1-allylquinolinium, and 8-formyl-1-allyl-quinolinium, 5-acetyl-1-methyl-quinolinium, 6-acetyl-1-methyl-quinolinium, 7-acetyl-1-methyl-quinolinium, 8-acetyl-1-methyl-quinolinium, 5-acetyl-1-ethyl-quinolinium, 6-acetyl-1-ethyl-quinolinium, 7-acetyl-1-ethyl-quinolinium, 8-acetyl-1-ethyl-quinolinium, 5-acetyl-1-benzylquinolinium, 6-acetyl-1-benzylquinolinium, 7-acetyl-1-benzylquinolinium, 8-acetyl-1-benzylquinolinium, 5-acetyl-1-allyl-quinolinium, 6-acetyl-1-allyl-quinolinium, 7-acetyl-1-allyl-quinolinium, and 8-acetyl-1-allyl-quinolinium, 9-formyl-10-methylacridinium, 4-(2′-formylvinyl)-1-methylpyridinium, 1,3-dimethyl-2-(4′-formylphenyl)-benzimidazolium, 1,3-dimethyl-2-(4′-formylphenyl)-imidazolium, 2-(4′-formylphenyl)-3-methylbenzothiazolium, 2-(4′-acetylphenyl)-3-methylbenzothiazolium, 2-(4′-formylphenyl)-3-methylbenzothiazolium, 2-(5′-formyl-2′-furyl)-3-methylbenzothiazolium, 2-(5′-formyl-2′-thienyl)-3-methylbenzothiazolium, 2-(3′-formylphenyl)-3-methylbenzothiazolium, 2-(4′-formyl-1-naphthyl)-3-methylbenzothiazolium, 5-chloro-2-(4′-formylphenyl)-3-methylbenzothiazolium, 2-(4′-formylphenyl)-3,5-dimethylbenzothiazolium benzenesulfonate, p-toluenesulfonate, methanesulfonate, perchlorate, sulfate, chloride, bromide, iodide, tetrachlorozincate, methylsulfate, trifluoromethanesulfonate, or tetrafluoroborate, isatin, 1-methyl-isatin, 1-allyl-isatin, 1-hydroxymethyl-isatin, 5-chloro-isatin, 5-methoxy-isatin, 5-nitro-isatin, 6-nitro-isatin, 5-sulfo-isatin, 5-carboxy-isatin, quinisatin, 1-methylquinisatin and arbitrary mixtures of the compounds named above.

Benzaldehyde, cinnamaldehyde and naphthaldehyde and their derivatives, especially with one or more hydroxy, alkoxy or amino substituents are quite specially preferred as the reactive carbonyl compound in the agents according to the invention. Compounds according to formula (Ca-1) are also preferred,

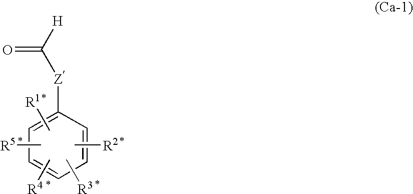

in which

R¹*, R²*, and R³*, independently of each other, stand for a hydrogen atom, a halogen atom, a C₁-C₆-alkyl group, a hydroxyl group, a C₁-C₆-alkoxy group, a C₁-C₆-dialkylamino group, a di(C₂-C₆)-hydroxyalkylamino group, a di(C₁-C₆)-alkoxy-C₁-C₆-alkyl)amino group, a C₁-C₆-hydroxyalkoxy group, a sulfonyl group, a carboxyl group, a sulfonic acid group, a sulfonamido group, a sulfonamide group, a carbamoyl group, a C₂-C₆-acyl group or a nitro group,

Z′ stands for a direct bond or a vinylene group,

R⁴* and R⁵* stand for a hydrogen atom, or, together with the rest of the molecule, form a 5-membered or 6-membered aromatic or aliphatic ring.

The derivatives of benzaldehyde, naphthaldehyde or cinnamaldehyde as the reactive carbonyl compound according to component C are especially preferably selected from certain aldehydes. Here, according to the invention, agents are preferred that contain, in addition, at least one reactive carbonyl compound selected from the group consisting of 4-hydroxy-3-methoxy-benzaldehyde, 3,5-dimethoxy-4-hydroxy-benzaldehyde, 4-hydroxy-1-naphthaldehyde, 4-hydroxy-2-methoxy-benzaldehyde, 3,4-dihydroxy-5-methoxy-benzaldehyde, 3,4,5-trihydroxybenzaldehyde, 3,5-dibromo-4-hydroxy-benzaldehyde, 4-hydroxy-3-nitro-benzaldehyde, 3-bromo-4-hydroxy-benzaldehyde, 4-hydroxy-3-methyl-benzaldehyde, 3,5-dimethyl-4-hydroxy-benzaldehyde, 5-bromo-4-hydroxy-3-methoxy-benzaldehyde, 4-diethylamino-2-hydroxy-benzaldehyde, 4-dimethylamino-2-methoxy-benzaldehyde, coniferyl aldehyde, 2-methoxy-benzaldehyde, 3-methoxy-benzaldehyde, 4-methoxy-benzaldehyde, 2-ethoxy-benzaldehyde, 3-ethoxy-benzaldehyde, 4-ethoxy-benzaldehyde, 4-hydroxy-2,3-di-methoxy-benzaldehyde, 4-hydroxy-2,5-di-methoxy-benzaldehyde, 4-hydroxy-2,6-dimethoxybenzaldehyde, 4-hydroxy-2-methyl-benzaldehyde, 4-hydroxy-2,3-dimethyl-benzaldehyde, 4-hydroxy-2,5-dimethyl-benzaldehyde, 4-hydroxy-2,6-dimethyl-benzaldehyde, 3,5-di-ethoxy-4-hydroxy-benzaldehyde, 2,6-di-ethoxy-4-hydroxy-benzaldehyde, 3-hydroxy-4-methoxy-benzaldehyde, 2-hydroxy-4-methoxy-benzaldehyde, 2-ethoxy-4-hydroxy-benzaldehyde, 3-ethoxy-4-hydroxy-benzaldehyde, 4-ethoxy-2-hydroxy-benzaldehyde, 4-ethoxy-3-hydroxy-benzaldehyde, 2,3-di-methoxy-benzaldehyde, 2,4-dimethoxy-benzaldehyde, 2,5-dimethoxy-benzaldehyde, 2,6-dimethoxy-benzaldehyde, 3,4-dimethoxy-benzaldehyde, 3,5-dimethoxy-benzaldehyde, 2,3,4-trimethoxy-benzaldehyde, 2,3,5-trimethoxy-benzaldehyde, 2,3,6-trimethoxy-benzaldehyde, 2,4,6-trimethoxy-benzaldehyde, 2,4,5-trimethoxy-benzaldehyde, 2,5,6-trimethoxy-benzaldehyde, 2-hydroxy-benzaldehyde, 3-hydroxy-benzaldehyde, 4-hydroxy-benzaldehyde, 2,3-di-hydroxy-benzaldehyde, 2,4-di-hydroxy-benzaldehyde, 2,4-di-hydroxy-3-methyl-benzaldehyde, 2,4-di-hydroxy-5-methyl-benzaldehyde, 2,4-dihydroxy-6-methyl-benzaldehyde, 2,4-dihydroxy-3-methoxy-benzaldehyde, 2,4-dihydroxy-5-methoxybenzaldehyde, 2,4-dihydroxy-6-methoxybenzaldehyde, 2,5-dihydroxy-benzaldehyde, 2,6-dihydroxybenzaldehyde, 3,4-dihydroxy-benzaldehyde, 3,4-dihydroxy-2-methyl-benzaldehyde, 3,4-dihydroxy-5-methyl-benzaldehyde, 3,4-dihydroxy-6-methyl-benzaldehyde, 3,4-dihydroxy-2-methoxy-benzaldehyde, 3,5-dihydroxy-benzaldehyde, 2,3,4-trihydroxy-benzaldehyde, 2,3,5-trihydroxy-benzaldehyde, 2,3,6-trihydroxy-benzaldehyde, 2,4,6-trihydroxybenzaldehyde, 2,4,5-trihydroxy-benzaldehyde, 2,5,6-trihydroxy-benzaldehyde, 4-dimethylamino-benzaldehyde, 4-diethylamino-benzaldehyde, 4-dimethylamino-2-hydroxy-benzaldehyde, 4-pyrrolidino-benzaldehyde, 4-morpholino-benzaldehyde, 2-morpholino-benzaldehyde, 4-piperidino-benzaldehyde, 3,5-dichloro-4-hydroxy-benzaldehyde, 4-hydroxy-3,5-diiodo-benzaldehyde, 3-chloro-4-hydroxy-benzaldehyde, 5-chloro-3,4-di-hydroxy-benzaldehyde, 5-bromo-3,4-di-hydroxy-benzaldehyde, 3-chloro-4-hydroxy-5-methoxy-benzaldehyde, 4-hydroxy-3-iodo-5-methoxy-benzaldehyde, 2-methoxy-1-naphthaldehyde, 4-methoxy-1-naphthaldehyde, 2-hydroxy-1-naphthaldehyde, 2,4-di-hydroxy-1-naphthaldehyde, 4-hydroxy-3-methoxy-naphthadehyde, 2-hydroxy-4-methoxy-1-naphthaldehyde, 3-hydroxy-4-methoxy-1-naphthaldehyde, 2,4-di-methoxy-1-naphthaldehyde, 3,4-di-methoxy-1-naphthaldehyde, 4-dimethylamino-1-naphthaldehyde, 2-nitro-benzaldehyde, 3-nitro-benzaldehyde, 4-nitro-benzaldehyde, 4-methyl-3-nitro-benzaldehyde, 3-hydroxy-4-nitro-benzaldehyde, 5-hydroxy-2-nitro-benzaldehyde, 2-hydroxy-5-nitro-benzaldehyde, 2-hydroxy-3-nitro-benzaldehyde, 2-fluoro-3-nitro-benzaldehyde, 2-methoxy-2-nitro-benzaldehyde, 4-chloro-2-nitro-benzaldehyde, 2-chloro-6-nitro-benzaldehyde, 5-chloro-2-nitro-benzaldehyde, 4-chloro-3-nitro-benzaldehyde, 2,4-dinitro-benzaldehyde, 2,6-dinitro-benzaldehyde, 2-hydroxy-3-methoxy-5-nitro-benzaldehyde, 4,5-di-methoxy-2-nitro-benzaldehyde, 6-nitropiperonal, 2-nitropiperonal, 5-nitrovanillin, 2,5-dinitrosalicylaldehyde, 5-bromo-3-nitrosalicylaldehyde, 4-nitro-1-naphthaldehyde, 2-nitrocinnamaldehyde, 3-nitrocinnamaldehyde, 4-nitrocinnamaldehyde, 4-dimethylamino-cinnamaldehyde, 2-dimethylamino-benzaldehyde, 2-chlor-4-dimethylamino-benzaldehyde, 4-dimethylamino-2-methyl-benzaldehyde, 4-diethylamino-cinnamaldehyde, 4-dibutylamino-benzaldehyde, 4-diphenylamino-benzaldehyde, 4-(1-imidazolyl)-benzaldehyde and piperonal.

Agents preferred according to the invention are characterized by being formulated as hair-colorants comprising, based on its weight, 0.01 to 20% by weight, preferably 0.05 to 15% by weight, especially preferably 0.1 to 12.5% by weight, and particularly 0.2 to 10% by weight of one or more reactive carbonyl compounds.

In general, CH acids are considered to be those compounds having a hydrogen atom bonded to an aliphatic carbon atom, in which the corresponding carbon-hydrogen bond is activated by electron-withdrawing substituents. According to the invention, enamines, produced by alkaline treatment of quaternized N-heterocycles having a CH-acid alkyl group conjugated with the quaternary nitrogen are also CH acids.

The CH acid compounds of component B are preferably selected from the group comprising 1,2,3,3-tetramethyl-3H-indolium iodide, 1,2,3,3-tetramethyl-3H-indolium p-toluenesulfonate, 1,2,3,3-tetramethyl-3H-indolium methanesulfonate, 1,3,3-trimethyl-2-methyleneindoline (Fisher's base), 2,3-dimethylbenzothiazolium iodide, 2,3-dimethylbenzothiazolium p-toluenesulfonate, 2,3-dimethyl-naphtho[1,2-d]thiazolium p-toluenesulfonate, 3-ethyl-2-methyl-naphtho[1,2-d]thiazolium p-toluenesulfonate, rhodanine, rhodanin-3-acetic acid, 1,4-dimethyl-quinolinium iodide, 1,2-dimethyl-quinolinium iodide, barbituric acid, thiobarbituric acid, 1,3-dimethylbarbituric acid, 1,3-dimethylthiobarbituric acid, 1,3-diethylthiobarbituric acid, 1,3-diethylbarbituric acid, oxindole, 3-indoxyl acetate, 2-coumaranone, 5-hydroxy-2-coumaranone, 6-hydroxy-2-coumaranone, 3-methyl-1-phenyl-pyrazolin-5-one, indan-1,2-dione, indan-1,3-dione, indan-1-one, benzoylacetonitrile, 3-dicyanomethyleneindan-1-one, 2-amino-4-imino-1,3-thizaoline hydrochloride, 5,5-dimethylcyclohexan-1,3-dione, 2H-1,4-benzoxazin-4H-3-one, 3-ethyl-2-methylbenzoxazolium iodide, 3-ethyl-2-methyl-benzothiazolium iodide, 1-ethyl-4-methyl-quinolinium iodide, 1-ethyl-2-methyl-quinolinium iodide, 1,2,3-trimethylquinolinium iodide, 3-ethyl-2-methyl-benzoxazolium p-toluenesulfonate, 3-ethyl-2-methyl-benzothiazolum p-toluenesulfonate, 1-ethyl-4-methyl-quinolinium p-toluenesulfonate, 1-ethyl-2-methyl-quinolonium p-toluenesulfonate and 1,2,3-trimethyl-quinoxalinium p-toluenesulfonate.

Examples of suitable compounds with a primary or secondary amino group as component B include, for example, primary aromatic amines such as N,N-dimethyl, N,N-diethyl, N-(2-hydroxyethyl)-N-ethyl-, N,N-bis-(2-hydroxyethyl), N-(2-methoxyethyl), 2,3, 2,4, and 2,5-dichloro-p-phenylendiamine, 2-chloro-p-phenylendiamine, 2,5-dihydroxy-4-morpholinoaniline dihydrobromide, 2-, 3-, or 4-aminophenol, 2-aminomethyl-4-aminophenol, 2-hydroxymethyl-4-aminophenol, o-phenylendiamine, p-phenylendiamine, o-toluoylenediamine, 2,5-diaminotoluene, 2,5-diaminophenol, -phenethol, 4-amino-3-methylphenol, 2-(2,5-diaminophenyl)ethanol, 2,4-diaminophenoxyethanol, 2-(2,5-diaminophenoxy)ethanol, 4-methylamino-, 3-amino-4-(2′-hydroxyethoxy)-, 3,4-methylenediamino-, 3,4-methylenedioxyaniline, 3-amino-2,4-dichloro-, 4-methylamino-, 2-methyl-5-amino-, 3-methyl-4-amino-, 2-methyl-5-(2-hydroxyethylamino)-, 6-methyl-3-amino-2-chloro-, 2-methyl-5-amino-4-chloro-, 3,4-methylendioxy-, 5-(2-hydroxyethylamino)-4-methoxy-2-methyl-, 4-amino-2-hydroxymethylphenol, 1,3-diamino-2,4-dimethoxybenzene, 2-, 3-, or 4-amino-benzoic acid, -phenylacetic acid, 2,3-, 2,4-, 2,5- 3,4-, 3,5-diaminobenzoic acid, 4- or 5-aminosalicylic acid, 3-amino-4-hydroxy-, 4-amino-3-hydroxy-benzoic acid, 2-, 3-, or 4-aminobenzenesulfonic acid, 3-amino-4-hydroxybenzenesulfonic acid, 4-amino-3-hydroxy-napthalene-1-sulfonic acid, 6-amino-7-hydroxy-naphthalene-2-sulfonic acid, 7-amino-4-hydroxy-naphthalene-2-sulfonic acid, 4-amino-5-hydroxy-naphthalene-2,7-disulfonic acid, 3-amino-2-naphthoic acid, 3-aminophthalic acid, 5-amino-isophthalic acid, 1,3,5- or 1,2,4-triaminobenzene, 1,2,4,5-tetraaminobenzene, 2,4,5-triaminophenol, pentaaminobenzene, hexaaminobenzene, 2,4,6-triaminoresorcinol, 4,5-diamino-pyrocatechol, 4,6-diaminopyrogallol, 3,5-diamino-4-hydroxy-pyrocatechol, aromatic anilines or phenols with another aromatic group, such as indicated in the formula below

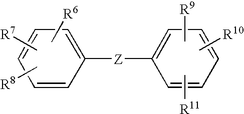

in which R⁶ stands for a hydroxy or an amino group that can be substituted by C₁₋₄ alkyl, C₁₋₄ hydroxyalkyl, or C₁₋₄-alkoxy-C₁₋₄-alkyl, R⁷, R⁸, R⁹, R¹⁰ and R¹¹ stand for hydrogen, a hydroxy or an amino group that can be substituted by a C₁₋₄-alkyl, C₁₋₄-hydroxy-alkyl, C₁₋₄-aminoalkyl, or C₁₋₄-alkoxy-C₁₋₄-alkyl group, stand for a carboxylic acid or sulfonic acid group, and Z stands for a direct bond, a saturated or unsaturated carbon chain with 1 to 4 carbon atoms, perhaps substituted by hydroxy groups, a carbonyl, sulfonyl or imino group, an oxygen or a sulfur atom, or a group with the formula 111 Q-(CH₂-p-CH₂-Q′)1>(111) in which P means a direct bond or a CH₂ or CHOH group, Q and Q′, independently of each other, stand for an oxygen atom, a NR¹² group, in which R¹² is hydrogen, or a chloro-, alkyl-, or hydroxy-C₁₋₄-alkyl group, the group —O—(CH₂)p-NH— or —NH—(CH₂)p-O—, in which p and p′ are 2 or 3, and o is a number from 1 to 4, such as 4,4′-diaminostilbene, 4,4′-diaminostilbene-2,2′-disulfonic acid mono-sodium or di-sodium salt, 4-amino-4′dimethylaminostilbene, 4,4′-diaminodiphenylmethane, sulfide, sulfoxide, amine, 4,4′-diaminodiphenylamine-2-sulfonic acid, 4,4′-diaminobenzophenone diphenyl ether, 3,3′,4,4′-tetraaminodiphenyl, 3,3′,4,4′-tetraaminobenzophenone, 1,3-bis-(2,4-diaminohenoxy)-propane, 1,8-bis-(2,5-diaminophenoxy)-3,6-dioxaoctane, 1,3-bis-(4-aminophenylamino)-propane, or -2-propanol, 1,3-bis-[-(4-aminophenyl)-2-hydroxyethylamino]-2-propanol, N,N-bis[2-(4-aminophenoxy)-ethyl]methylamine, or N-phenyl-1,4-phenylene-diamine;

The compounds named above can be used both in the free form and in the form of their physiologically acceptable salts, especially as salts of inorganic acids, such as hydrochloric acid or sulfuric acid.

Examples of suitable nitrogen-containing heterocyclic compounds include 2-, 3-, or 4-amino, 2-amino-3-hydroxy-, 2,6-diamino-, 2,5-diamino, 2,3-diamino, 2-dimethylamino-5-amino-, 2-methylamino-3-amino-6-methoxy-, 2,4-diamino-6-methoxy-, 2,6-dimethoxy-, 3,5-diamino-, 2,4,5-triamino-, 2,6-dihydroxy-3,4-dimethyl-pyridine, 2,4-di-hydroxy-, 5,6-diamino-, 4,5,6-triamino, 4-hydroxy-2,5,6-triamino-, 2-hydroxy-4,5,6-triamino-, 2,4,5,6-tetramino-, 2-methylamino-4,5,6-triamino-, 2,4-, 4,5-diamino, 2-amino-4-methoxy-6-methylpyrimidine, 3,5-diaminopyrazole, -1,2,4-triazole, 3-amino-, 3-amino-5-hydroxy-pyrazole, 2-, 3- or 8-aminoquinoline, 4-aminoquinaldine, 2- or 6-amino-nicotinic acid, 5-amino-isoquinoline, 5, or 6-aminoindazole, 5- or 7-aminobenzimidazole, -benzothiazol, 2,5-dihydroxy-4-morpholinoaniline and indole and indoline, and their physiologically acceptable salts. Preferred examples of indole or indoline derivatives [are] 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole and 4-aminoindole. Further preferred compounds are 5,6-dihydroxyindoline, N-methyl-5,6-dihydroxyidoline, N-ethyl-5,6-dihydroxyindoline, N-propyl-5,6-dihyxroxyindoline, N-butyl-5,6-dihydroxyindoline, 5,6-dihydroxyindoline-2-carboxylic acid, 6-hydroxyindoline, 6-aminoindoline, and 4-aminoindoline. The compounds named above can be used both in the free form and also in the form of their physiologically acceptable salts, e.g., as salts of inorganic acids, such as hydrochloric acid or sulfuric acid.

All the naturally occurring and synthetic alpha-amino acids can be considered as amino acids, for example, the amino acids accessible by hydrolysis of plant or animal proteins, such as collagen, keratin, casein, elastin, soy protein, wheat gluten or almond protein. Both acidic and alkaline amino acids can be used. Preferred amino acids are arginine, histidine, tyrosine, phenylalanine, DOPA (dihydroxyphenylalanine), ornithine, lysine and tryptophan. Other amino acids, such as 6-aminocaproic acid, can also be used.

The oligopeptides can be naturally occurring or synthetic oligopeptides, as well as the oligopeptides contained in polypeptide or protein hydrolyzates, as long as they have enough water solubility for use in the colorants according to the invention, protein hydrolyzates, as long as they have enough water solubility for use in the colorants according to the invention. Glutathione or the oligopeptides contained in the hydrolysates of collagen, keratin, casein, elastin, soy protein, wheat gluten or almond protein can be named as examples. It is preferred to use them together with compounds having primary or secondary amino groups or with aromatic hydroxy compounds.

Examples of suitable aromatic hydroxy compounds include 2-, 4-, or 5-methylresorcinol, 2,5-dimethylresorcinol, resorcinol, 3-methoxyphenol, pyrocatechol, hydroquinone, pyrogallol, phloroglucinol, hydroxy-hydroquinone, 2-, 3-, or 4-methoxy-, 3-dimethylamino-, 2-(2-hydroxyethyl)-, 3,4-methylenedioxyphenol, 2,4- or 3,4-dihydroxybenzoic acid, -phenylacetic acid, gallic acid, 2,4,6-trihydroxybenzoic acid, -acetophenone, 2- or 4-chlororesorcinol, 1-naphthol, 1,5-, 2,3- or 2,7-dihydroxynaphthalene, 6-dimethylamino-4-hydroxy-2-naphthalenesulfonic acid, and 3,6-dihydroxy-2,7-naphthalene[-di]sulfonic acid.

Examples of CH-active compounds include 1,2,3,3-tetramethyl-3H-indolium iodide, 1,2,3,3-tetramethyl-3H-indolium p-toluenesulfonate, 1,2,3,3-tetramethyl-3H-indolium methanesulfonate, Fischer's base (1,3,3-trimethyl-2-methylene-indoline, 2,3-dimethylbenzothiazolium iodide, 2,3-dimethylbenzothiazolium p-toluenesulfonate, rhodanine, rhodanine-3-acetic acid, 1-ethyl-2-quinaldinium iodide, 1-methyl-2-quinaldinium iodide, barbituric acid, thiobarbituric acid, 1,3-dimethylthiobarbituric acid, diethylthiobarbituric acid, oxindole, 3-indoxyl acetate, coumarone and 1-methyl-3-phenyl-2-pyrazolinone.

The CH-acidic compounds are selected preferably from formulas (II) and/or (III) and/or (IV).

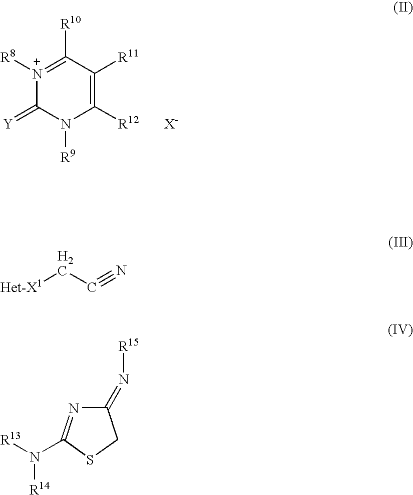

in which
R⁸ and R⁹, independently of each other, stand for a linear or cyclic C₁-C₆-alkyl group, a C₂-C₆-alkenyl group, an optionally substituted aryl group, an optionally substituted heteroaryl group, an aryl-C₁-C₈-alkyl group, a C₁-C₆-hydroxyalkyl group, a C₂-C₆-polyhydroxyalkyl group, a C₁-C₆-alkoxy-C₁-C₆-alkyl group, a group R^{I}R^{II}N—(CH₂)ₘ—, in which R^{I} and R^{II}, independently of each other, stand for a hydrogen atom a (C₁-C₄)-alkyl group, a (C₁-C₄)-hydroxyalkyl group or an aryl-C₁-C₆-alkyl group, in which R^{I} and R^{II}, together with the nitrogen atom can form a 5-, 6- or 7-membered ring and m stands for a number 2, 3, 4, 5 or 6,
R¹⁰ and R¹², independently of each other, stand for a hydrogen atom or for a C₁-C₆-alkyl group, with at least one of the groups R¹⁰ and R¹² being a C₁-C₆-alkyl group,
R¹¹ stands for a hydrogen atom, a C₁-C₆-alkyl group, a C₁-C₆-hydroxyalkyl group, a C₂-C₆-polyhydroxyalkyl group, a C₁-C₆-alkoxy group, a C₁-C₆-hydroxyalkoxy group, a group R^{III}R^{IV}N—(CH₂)_{q}—, in which R^{III} and R^{IV}, independently of each other, stand for a hydrogen atom, a C₁-C₆-alkyl group, a C₁-C₆-hydroxyalkyl group, or an aryl-C₁-C₆-alkyl group, and q stands for a number 1, 2, 3, 4, 5, or 6, in which the group R¹¹ together with one of the groups R¹⁰ or R¹² can form a 5- or 6-membered aromatic ring, which can optionally be substituted with a halogen atom, a C₁-C₆-alkyl group, a C₁-C₆-hydroxyalkyl group, a C₂-C₆-polyhydroxyalkyl group, a C₁-C₆-alkoxy group, a C₁-C₆-hydroxyalkoxy group, a nitro group, a hydroxy group, a group R^{V}R^{VI}N—(CH₂)ₛ—, in which R^{V} and R^{VI}, independently of each other, stand for a hydrogen atom, a C₁-C₆-alkyl group, a C₁-C₆-hydroxyalkyl group or an aryl-C₁-C₆-alkyl group and s stands for a number 0, 1, 2, 3, 4, 5, 6,
R¹³ and R¹⁴ either form a saturated or unsaturated 5- or 6-membered ring together with the nitrogen atom, or, independently of each other, stand for a (C₁-C₆)-alkyl group, a (C₂-C₆)-alkenyl group, an aryl group, an aryl-(C₁-C₆)-alkyl group, a (C₂-C₆)-hydroxyalkyl group, a (C₂-C₆)-polyhydroxyalkyl group, or a group R¹R^{II}N—(CH₂)ₘ—, in which R^{I} and R^{II}, independently of each other, stand for a hydrogen atom, a (C₁-C₆)-alkyl group, a (C₁-C₆)-alkenyl group or an aryl-C₁-C₆-alkyl group, with R^{I} and R^{II} being able to form a 5-, 6- or 7-membered ring, together with the nitrogen atom, and m stands for a number 2, 3, 4, 5, or 6,
R¹⁵ is a hydrogen atom, a (C₁-C₆)-alkyl group, a (C₂-C₆)-alkenyl group, an aryl group, an aryl-(C₁-C₆)-alkyl group, a (C₂-C₆)-hydroxylalkyl group, a (C₂-C₆)-polyhydroxyalkyl group or a group R^{III}R^{IV}N—(CH₂)ₙ—, in which R^{III} and R^{IV}, independently of each other, stand for a hydrogen atom, a (C₁-C₆)-alkyl group, a (C₁-C₆)-alkenyl group or an aryl-C₁-C₆-alkyl group, with R^{III} and R^{IV} being able to form a 5-, 6-, or 7-membered ring, together with the nitrogen atom, and n stands for a number 2, 3, 4, 5 or 6,
Y stands for an oxygen atom, a sulfur atom, or a group NR^{VII}, in which R^{VII} stands for a hydrogen atom, an aryl group, a heteroaryl group, a C₁-C₆-alkyl group or a C₁-C₆-arylalkyl group,
X⁻ stands for a physiologically acceptable anion,
Het stands for an optionally substituted heteroaromatic,
X¹ stands for a direct bond or for a carbonyl group.

The enamine forms of the compounds of formula 2 have the same effects. Reference is expressly made here to the complete contents of patent WO-A1-2004/022016.

At least one group R¹⁰ or R¹² must stand for a C₁-C₆-alkyl group. This alkyl group preferably has at least two hydrogen atoms on its alpha carbon atom. Specially preferred alkyl groups are the methyl, ethyl, propyl, n-butyl-, iso-butyl-, n-pentyl-, neo-pentyl-, and n-hexyl groups. It is quite specially preferable for R¹⁰ and R¹², independently of each other, to stand for hydrogen or a methyl group, with at least one group R¹⁰ or R¹² being a methyl group.

In a preferred embodiment, Y stands for an oxygen or a sulfur atom, especially preferably for an oxygen atom.

The group R⁸ is preferably selected from a (C₁-C₆)-alkyl group (especially preferably a methyl group), a C₂-C₆-alkenyl group (especially an allyl group), a hydroxy-(C₂-C₆)-alkyl group, especially a 2-hydroxyethyl group, or an optionally substituted benzyl group.

R11 preferably stands for a hydrogen atom.

It is specially preferred for the groups R⁹, R¹⁰ and R¹² to stand for methyl groups, the group R¹¹ to stand for a hydrogen atom, Y for an oxygen or sulfur atom, and the group R⁸ is, selected from a (C₁-C₆)-alkyl group (especially preferably a methyl group), a C₂-C₆-alkenyl group (especially an allyl group), a hydroxy-(C₂-C₆)-alkyl group, especially a 2-hydroxyethyl group, or an optionally substituted benzyl group.

The compounds according to formula II are preferably selected from one or more compounds of the group of salts with physiologically acceptable counterion X—, formed of salts of
1,2-dihydro-1,3,4,6-tetramethyl-2-oxo-pyrimidinum,
1,2-dihydro-1,3-diethyl-4,6-dimethyl-2-oxo-pyrimidinum,
1,2-dihydro-1,3-dipropyl-4,6-dimethyl-2-oxo-pyrimidinum,
1,2-dihydro-1,3-di(2-hydroxyethyl)-4,6-dimethyl-2-oxo-pyrimidinum,
1,2-dihydro-1,3-diphenyl-4,6-dimethyl-2-oxo-pyrimidinum,
1,2-dihydro-1,3,4-trimethyl-2-oxo-pyrimidinum
1,2-dihydro-1,3-diethyl-4-methyl-2-oxo-pyrimidinum
1,2-dihydro-1,3-dipropyl-4-methyl-2-oxo-pyrimidinum,
1,2-dihydro-1,3-di-(2-hydroxyethyl)4-methyl-2-oxo-pyrimidinum,
1,2-dihydro-1,3-diphenyl-4-methyl-2-oxo-pyrimidinum,
1-allyl-1,2-dihydro-3,4,6-trimethyl-2-oxo-pyrimidinum,
1,2-dihydro-1-(2-hydroxyethyl)-3,4,6-trimethyl-2-oxo-pyrimidinum,
1,2-dihydro-1,3,4,6-tetramethyl-2-thioxo-pyridinium,
1,2-dihydro-1,3-diethyl-4,6-dimethyl-2-thioxo-pyridinium,
1,2-dihydro-1,3-dipropyl-4,6-dimethyl-2-thioxo-pyridinium,
1,2-dihydro-1,3-di(2-hydroxyethyl)-4,6-dimethyl-2-thioxo-pyridinium,
1,2-dihydro-1,3-diphenyl-4,6-dimethyl-2-thioxo-pyridinium,
1,2-dihydro-1,3,4-trimethyl-2-thioxo-pyridinium,
1,2-dihydro-1,3-diethyl-4-methyl-2-thioxo-pyridinium,
1,2-dihydro-1,3-dipropyl-4-methyl-2-thioxo-pyridinium,
1,2-dihydro-1,3-di(2-hydroxyethyl)-4-methyl-2-thioxo-pyridinium,
1,2-dihydro-1,3-diphenyl-4-methyl-2-thioxo-pyridinium,
1,2-dihydro-3,4-dimethyl-2-oxo-quinazolinium and
1,2-dihydro-3,4-dimethyl-2-thioxo-quinazolinium.

Quite specially preferred agents according to the invention are characterized by comprising as the CH-acid compound
salts of 1,2-dihydro-1,3,4,6-tetramethyl-2-oxo-pyridinium,
salts of 1,2-dihydro-1,3,4-trimethyl-2-oxo-pyrimidinum,
salts of 1,2-dihydro-1,3,4,6-tetramethyl-2-thioxo-pyridinium,
salts of 1-allyl-1,2-dihydro-3,4,6-trimethyl-2-oxo-pyrimidinum,
salts of 1,2-dihydro-1-(2-hydroxyethyl)-3,4,6-trimethyl-2-oxo-pyrimidinum,
2-(cyanomethyl)-benzimidazole,
4,5-dihydro-4-imino-2-(1-piperidinyl)-thiazole and/or its hydrochloride,
4,5-dihydro-4-imino-2-(4-morpholinyl)-thiazole and/or its hydrochloride,
4,5-dihydro-4-imino-2-(1-pyrrolidinyl)-thiazole and/or its hydrochloride.

In formula (II) and in the preceding lists, X— stands preferably for halide, benzenesulfonate, p-toluenesulfonate, C₁-C₄-alkane sulfonate, trifluoromethanesulfonate, perchlorate, 0.5 sulfate, bisulfate, tetrafluoroborate, hexafluorophosphate or tetrachlorozincate. The anions chloride, bromide, iodide, bisulfate or p-toluenesulfonate are used preferably as X.

The group Het according to formula III preferably stands for the molecular fragment having the formula (V),

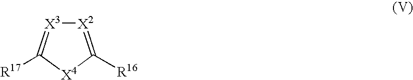

in which

R¹⁶ and R¹⁷, independently of each other, stand for a hydrogen atom, a hydroxy group, a halogen atom, a nitro group, a linear or cyclic C₁-C₆-alkyl group, a C₂-C₆-alkenyl group, an optionally substituted aryl group, a cyanomethyl group, a cyanomethylcarbonyl group, an optionally substituted heteroaryl group, an aryl-C₁-C₆-alkyl group, a C₁-C₆-hydroxyalkyl group, a C₂-C₆-polyhydroxyalkyl group, a C₁-C₆-alkoxy group, a C₁-C₆-alkoxycarbonyl group, a C₁-C₆-alkoxy-C₂-C₆-alkyl group, a C₁-C₆-sulfoalkyl group, a C₁-C₆-carboxyalkyl group, a group R^{VIII}R^{IX}N—(CH₂)ₘ—, in which R^{VIII} and R^{IX}, independently of each other, stand for a hydrogen atom, a linear or cyclic C₁-C₆-alkyl group, a C₂-C₆-alkenyl group, a C₁-C₆-hydroxyalkyl group or an aryl-C₁-C₄-alkyl group, in which R^{VIII} and R^{IX}, together with the nitrogen atom, can form a 5-, 6-, or 7-membered ring and m stands for a number 0, 1, 2, 3, or 4, in which R¹⁸ and R¹⁷ can form an optionally substituted aromatic or heteroaromatic 5- or 6-membered ring condensed to the ring of the rest of the molecule,
X² and X³, independently of each other, stand for a nitrogen atom or for a group CR¹⁵, in which R¹⁵ stands for a hydrogen atom, a hydroxy group, a halogen atom, a nitro group, a linear or cyclic C₁-C₆-alkyl group, a C₂-C₆-alkenyl group, an optionally substituted aryl group, a cyanomethyl group, a cyanomethylcarbonyl group, an optionally substituted heteroaryl group, an aryl-C₁-C₆-alkyl group, a C₁-C₆-hydroxyalkyl group, a C₂-C₆-polyhydroxyalkyl group, a C₁-C₆-alkoxy group, a C₁-C₆-alkoxycarbonyl group, a C₁-C₆-alkoxy-C₂-C₆-alkyl group, a C₁-C₆-sulfoalkyl group, a C₁-C₆-carboxyalkyl group, a group R^{X}R^{XI}iN—(CH₂)ₙ—, in which R^{X} and R^{XI}, independently of each other, stand for a hydrogen atom, a linear or cyclic C₁-C₆-alkyl group, a C₂-C₆-alkenyl group, a C₁-C₆-hydroxyalkyl group or an aryl-C₁-C₄-alkyl group, in which R^{X} and R^{XI}, together with the nitrogen atom, can form a 5-, 6-, or 7-membered ring and m stands for a number 0, 1, 2, 3, or 4, in which at least one of the substituents X² and X³, together with the remainder of the molecule can form a condensed-on optionally substituted aromatic 5- or 6-membered ring,
X⁴ stands for an oxygen atom, a sulfur atom, a vinylene group or a group N—H, with the last two groups named, independently of each other, optionally having a linear or cyclic C₁-C₆-alkyl group, a C₂-C₆-alkenyl group, an optionally substituted aryl group, an optionally substituted heteroaryl group, an aryl-C₁-C₆-alkyl group, a C₂-C₆-hydroxyalkyl group, a C₂-C₆-polyhydroxylalkyl group, a C₁-C₆-alkoxy-C₂-C₆-alkyl group, a C₁-C₆-sulfoalkyl group, a C₁-C₆-carboxyalkyl group, a group R^{XII}R^{XIII}N—(CH2)p-, in which R^{XII} and R_{XIII}, independently of each other, stand for a hydrogen atom, a linear or cyclic C₁-C₆-alkyl group, a C₂-C₆-alkenyl group, a C₁-C₆-hydroxyalkyl group or an aryl-C₁-C₄-alkyl group, in which R^{XII} and R_{XIII}, together with the nitrogen atom, can form a 5-, 6-, or 7-membered ring and p stands for a number 0, 1, 2, 3, or 4,

provided that, if X⁴ stands for vinylene group, at least one of the groups X² or X³ is a nitrogen atom.

The binding of the heterocyclic ring according to formula (V) to the molecular fragment —X¹—CH₂—C≡N to get the compound of formula (III) according to the invention occurs at the heterocyclic ring, replacing a hydrogen atom bonded to that ring. Thus it is very necessary for the substituents R¹⁶, R¹⁷, X², X³ and X⁴ to be selected so that at least one of these substituents allows formation of the corresponding bond. Accordingly, it is essential that at least one of the groups R¹⁶ or R¹⁷ forms the bond to the molecular fragment —X¹—CH₂—C≡N, if X⁴ is an oxygen atom or a sulfur atom and X² and X³ are nitrogen atoms.

The group Het according to formula (III) is preferably derived from the heteroaromatics furan, thiophene, pyrrole, isoxazole, isothiazole, imidazole, oxazole, thiazole, pyridine, pyridazine, pyrimidine, pyrazine, 1,2,3-triazine, 1,2,4-triazine, 1,3,5-triazine, benzopyrrole, benzofuran, benzothiophene, benzimidazole, benzoxazole, indazol, benzoisoxazole, benzoisothiazole, indole, quinoline, isoquinoline, cinnoline, phthalazine, quinazoline, quinoxaline, acridine, benzoquinoline, benzo-isoquinoline, benzothiazole, phenazine, benzocinnoline, benzoquinazoline, benzoquinoxaline, phenoxazine, phenothiazine, naphthyridine, phenanthroline, indolizine, quinolizine, carboline, purine, pteridine and coumarine, in which the previously named heteroaromatics can be substituted by at least one group selected from a halogen atom, a nitro group, a thio group, a thio-(C₁-C₆)-alkyl group, a heteroaryl group, an aryl group, a (C₁-C₆)-alkyl group, a (C₁-C₆)-alkoxy group, a hydroxy group, a (C₂-C₆)-hydroxyalkyl group, a (C₂-C₆)-polyhydroxyalkyl group, a (C₁-C₆)-alkoxyl-(C₁-C₆)-alkyl group, an aryl-(C₁-C₆)-alkyl group, an amino group, a (C₁-C₆)-monoalkylamino group, a (C₁-C₆)-dialkylamino group, or a dialkylamino group —(CH₂)ₙ—NR′R″, in which n is an integer from 2 to 6 and R′ and R″, independently of each other, are a linear or branched alkyl group, which may optionally form a ring together.

The compounds according to formula (III) are preferably selected from the group consisting of 2-(2-furoyl)-acetonitrile, 2-(5-bromo-2-furoyl)acetonitrile, 2-(5-methyl-2-trifluoromethyl-3-furoyl-acetonitrile, 3-(2,5-dimethyl-3-furyl)-3-oxopropanenitrile, 2-(2-thenoyl)-acetonitrile, 2-(3-thenoyl)-acetonitrile, 2-(5-fluoro-2-thenoyl)-acetonitrile, 2-(5-chloro-2-thenoyl)-acetonitrile, 2-(5-bromo-2-thenoyl)-acetonitrile, 2-(5-methyl-2-thenoyl)-acetonitrile, -(2,5-dimethylpyrrol-3-oyl)-acetonitrile, 2-(1,2,5-trimethylpyrrol-3-oyl)-acetonitrile, 1H-benzimidazol-2-lyl acetonitrile, 1H-benzothiazol-2-yl-acetonitrile, 2-(pyrid-2-yl)-acetonitrile, 2,6-bis(cyanomethyl)-pyridine, 2-(3-indol-3-oyl)-acetonitrile, 2-(2-methyl-indol-3-oyl)-acetonitrile, 8-cyanoacetyl-7-methoxy-4-methylcoumarin, 2-(2-isopropyl-5,6-benzoquinolin-4-oyl)-acetonitrile, 2-(2-phenyl-5,6-benzoquinolin-4-oyl)-acetonitrile, 2-(quinoxalin-2-yl)-acetonitrile, 2-(coumaron-2-yl)-acetonitrile, 6,7-dichloro-5-(cyanoacetyl)-2,3-dihydro-1-benzofuran-2-carboxylic acid tert.-butyl ester, 2-(6-hydroxy-4,7-dimethoxy-1-benzofuran-5-oyl)-acetonitrile, and 2-(1-phenyl-1,4-dihydrothiochromenol[4,3-c]pyrazol-3-oyl)-acetonitrile. 1H-benzimidazol-2-yl-acetonitrile [2-(cyanomethyl)-benzimidazole] is specially preferred.

Agents according to the invention are distinguished by being formulated as hair colorants which comprise, based on its weight, 0.01 to 20% by weight, preferably 0.05 to 15% by weight, specially preferably 0.1 to 12.5% by weight and particularly 0.2 to 10% by weight of one or more CH-acid compounds.

Specially preferred agents according to the invention are produced as emulsions with such small particle sizes that the product appears “clear”. Preferred hair treatment agents are thus distinguished by being transparent or translucent.

The agents according to the invention exhibit advantageous properties and likewise impart advantageous properties to the body parts treated with them. Advantages are observed particularly for hair and scalp treatment. For instance, hair treatment agents according to the invention increase the elasticity of hairs treated with them and lead to strengthening of the internal structure of the hair fibers, as recorded, for instance, in higher melting temperatures in differential thermal analysis.

Improvements also appear in wet and dry combability and in prevention of premature splitting of the treated hairs.

On the skin, and especially the scalp, the agents according to the invention cause an increase in elasticity and surprising sebum-regulating effects. The optical impression of “greasy” skin or hair is thus avoided or diminished.

A further objective of the present invention is the use of hair treatment agents according to the invention for
increasing the elasticity of keratinic fibers, especially human hair and/or
improving the smoothness of keratinic fibers, especially human hair and/or
improving the gloss of keratinic fibers, especially human hair and/or
improving the wet and/or dry combability of keratinic fibers, especially human hair and/or
improving the protection of human hairs, especially human hair, from mechanical stress and/or
improving the deposition of silicones on keratinic fibers, especially human hair and/or
improving the deposition of care substances on keratinic fibers, especially human hair and/or
improving hair structure and/or
activating the scalp and/or
stimulating the scalp.

Products according to the invention can be formulated as the so-called “leave-on” products (hair lotions, hair treatments) or as the so-called “rinse-off” products (shampoo, rinsing conditioner, etc.). Depending on the product, then, a process for treating skin or hair in which a preparation is applied to the skin or hair, in which the preparation is rinsed off again after working time of 0 to 45 minutes, and a process for treating skin or hair in which a preparation is applied to the skin or hair and left there until the next shampoo are further objects of the present invention.

What has been said about the agents according to the invention applies mutatis mutandis to other preferred embodiments of the use according to the invention and the process according to the invention.

### EXAMPLES

Copolymerization of ca. 20 mole-% of partially neutralized acrylic acid and ca. 80 mole-% of a monomer of formula (Ia)

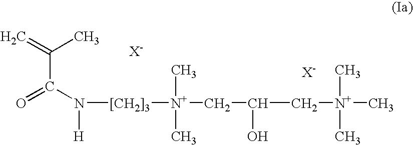

in which X stands for chloride, yields a copolymer A of the general formula (Ic

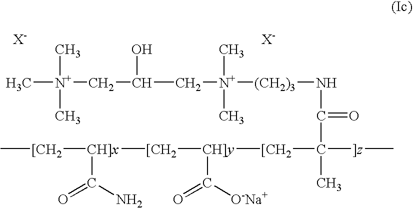

in which:
x stands for 0,
y stands for values of 50 to 100, especially for 70 to 80 and
z stands for values of 200 to 400, especially for 275 to 300.

The polymer is used in the following formulations:

All the data in the following tables are in % by weight if not otherwise specified.

### A) Shampoo

| | |
|---|---|
| Citric acid | 0.5 |
| Texapon ® NSO | 47.3 |
| Dehyton ® G | 17.0 |
| Salicylic acid | 0.2 |
| D-panthenol 75% | 0.2 |
| Sodium benzoate | 0.5 |
| Euperlan ® PK 3000 | 2.6 |
| Cetiol ® HE | 1.5 |
| Cutina ® HR | 0.5 |
| Dimethicone 500,000 | 2.0 |
| Polymer according to the example | 0.4 |
| Cetylstearyl alcohol with ca. 25 EO | 0.5 |
| units (INCI designation: Ceteareth-25) | |
| Sodium chloride | 0.2 |
| Water | to make 100 |

Texapon ® NSO Lauryl ether sulfate, sodium salt (ca. 27.5% active substance; INCI designation: Sodium laureth sulfate)(Cognis)
Dehyton ® G N-(2-hydroxyethyl)-N-cocoamidoethyl)-carboxymethyl-glycinate sodium salt (ca. 39-31% content of active substance; INCI designation: Aqua (water), disodium cocoamphodiacetate)((Cognis)
Euperlan ® PK3000 ca. 60-64% solids; INCI designation: Glycol distearate, glycerol, Laureth-4, cocamidopropyl betaine (Cognis)
Cetiol ® HE coco monoglyceride with ca. 7.3 EO units (INCI designation: PEG-7 Glyceryl cocoate) (Cognis)
Cutina ® HR hydrogenated castor oil (INCI designation: Hydrogenated castor oil)(Cognis)

### B) Hair Treatment:

| | |
|---|---|
| Pantolactone | 0.2 |
| Dehyquart ® F75 | 1.5 |
| Isopropyl myristate | 1.0 |
| Varisoft ® W 575 PG | 1.5 |
| Cetearyl alcohol | 4.0 |
| Caffeine | 0.1 |
| Dimethicone 500,000 | 2.0 |
| Cetylstearyl alcohol with ca. 9 EO | 0.8 |
| units (INCI designation: Ceteareth-9) | |
| Stearamidopropyl dimethylamine | 0.8 |
| Dehyquart ® A-CA | 3.0 |
| Citric acid | 0.4 |
| Methyl paraben | 0.2 |
| Phenoxyethanol | 0.2 |
| Polymer according to the example | 0.2 |
| Salcare ® SC 96 | 0.5 |
| Water | to make 100 |

Dehyquart ® F75 Fatty alcohol-methyltriethanolammonium methyl sulfate dialkyl ester mixture (INCI designation: Distearoylethyl hydroxyethylmonium methosulfate, Cetearyl alcohol)(Henkel)
Varisoft ® W 575 PG INCI designation: Quaternium-87 (Goldschmidt)
Dehyquart ® A-CA Trimethylhexadecylammonium chloride (ca. 24-26% active substance; INCI designation: Aqua (water), Cetrimonium chloride)(Cognis)
Salcare ® SC 96 ca. 50% active substance content; INCI designation: Polyquaternium-37, Propylene glycol dicaprylate/dicaprate, PPG-1 Trideceth-6 (CIBA)

### C) Hair Rinse

| | |
|---|---|
| Stenol ® 1618 | 7.0 |
| Genemin ® KDMP | 1.2 |
| Dehyquart ® F75 | 1.2 |
| Dimethicone 500,000 | 2.0 |
| Cetylstearyl alcohol with ca. 25 EO | 0.8 |
| units (INCI designation: Ceteareth-25) | |
| Methyl paraben | 0.2 |
| Phenoxyethanol | 0.4 |
| Caffeine | 0.1 |
| Polymer according to the example | 0.2 |
| Water | to make 100 |

Stenol ® 1618 C16-18 fatty alcohol (INCI designation: Cetearyl alcohol)(Cognis)
Genamin ® KDMP N,N,N-trimethyl-N-(C20-22-alkyl)ammonium chloride (ca. 77-83% active substance in isopropanol; INCI designation: Behentrimonium chloride)(Clariant)
Dehyquart ® F75 Fatty alcohol-methyltriethanolammonium methyl sulfate dialkyl ester mixture (INCI designation: Distearoylethyl hydroxyethylmonium methosulfate, Cetearyl alcohol)(Henkel)

### D) Hair Tonic:

| | |
|---|---|
| Pantolactone | 0.1 |
| Benzophenone-4 | 0.03 |
| Synthalen ® K | 0.24 |
| Neutrol ® TE | 0.25 |
| Dehyquart ® F75 | 1.5 |
| Ethanol 96% DEP denatured | 40.0 |
| Dimethicone 500,000 | 2.0 |
| Cetylstearyl alcohol with ca. 25 EO | 0.8 |
| units (INCI designation: Ceteareth-25) | |
| Menthol, natural | 0.03 |
| Polymer according to the example | 0.1 |
| Water | to make 100 |

Synthalen ® KPolyacrylic acid (ca. 89% active substance content; (INCI designation: Carbomer)(3V Sigma)
Neutrol ® TE N,N,N′,N′-tetrakis-(2-hydroxypropyl)-ethylenediamine (INCI designation: tetrahydroxypropyl ethylenediamine) (BASF)

1-35. (canceled)

36. A hair treatment agent comprising
A) a copolymer A of
A1) 0.1 to 50% (based on the total number of monomers in the copolymer) monomers having the formula (I) 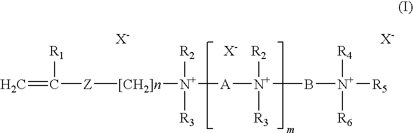
wherein
R¹ represents —H or —CH₃ or —CH₂CH₃
R², R³, R⁴, R⁵, R⁶ independently represent —H or —CH₃ or —CH₂CH₃ or —(CH₂)₂CH₃ or —CH(CH₃)₂ or —(CH₂)₃CH₃ or —CH(CH₃)CH₂CH₃ or —CH₂—CH(CH₃)₂ or —C(CH₃)₂ or —CH₂—OH or —CH₂)₂—OH or —CH(OH)—CH₃ or —CH(OH)—CH₂—CH₃ or —CH₂CH(OH)CH₃ or —CH₂)₃OH or —CH₂—NH₂ or —(CH₂)₂—NH₂ or —CH(NH₂)—CH₃ or —CH(NH₂)CH₂CH₃ or —CH₂CH(NH₂)CH₃ or —(CH₂)₃NH₂
n represents values of 1 to 6
m represents values of 0 to 10
Z represents —O— or —C(O)—O— or —C(O)—NH—
A represents —CH₂— or —(CH₂)₂— or —(CH₂)₃— or —(CH₂)₄— or —(CH₂)₅— or —(CH₂)₆—,
B represents a linear or branched C₂-C₁₂-chain that optionally may be interrupted by a heteroatom, and optionally may be substituted with a group selected from hydroxyl and amino, and
X represents an anion,
A2) represents a monomer selected from the group consisting of acrylic acid, methacrylic acid, alpha-ethylacrylic acid, beta,beta-dimethylacrylic acid, methylenemalonic acid, vinylacetic acid, allylacetic acid, ethylideneacetic acid, propylideneacetic acid, crotonic acid, maleic acid, fumaric acid, itaconic acid, citraconic acid, mesaconic acid, N-methacryloylalanine, N-acryloylhydroxyglycine, sulfopropyl acrylate, sulfoethyl acrylate, sulfoethyl methacrylate, sulfoethyl methacrylate, styrene sulfonic acid, vinylsulfonic acid, vinylphosphonic acid, phosphoethyl acrylate, phosphonoethyl acrylate, phosphopropyl acrylate, phosphonopropyl acrylate, phosphoethyl methacrylate, phosphonoethyl methacrylate, phosphopropyl methacrylate and phosphonopropyl methacrylate, and the alkali metal and ammonium salts of those acids,
A3) represents, optionally, a nonionic monomer selected from the group consisting of acrylamide, vinyl alcohol, C₁-C₄-alkyl esters of acrylic acid, C₁-C₄-alkyl esters of methacrylic acid, C₁-C₄-hydroxyalkyl esters of acrylic acid, C₁-C₄-hydroxyalkyl esters of methacrylic acid, polyalkoxylated esters of acrylic acid, polyalkoxylated esters of methacrylic acid, esters of acrylic acid, esters of methacrylic acid with polyethylene glycol or polypropylene glycol mono-(C₁-C₂₅-)-alkyl ethers, vinyl acetate, vinylpyrrolidone and methylvinyl ether, in which the monomers A2 and A3, together, make up 50% to 99.9% (based on the total number of monomers in the copolymer) of the copolymer;
B) a silicone, and
C) a care substance selected from the group consisting of
a) monomers, oligomers and polymers of amino acids, N—(C₂-C₂₄)-acylamino acids, the esters and the physiologically acceptable salts of these substances,
b) DNA or RNA oligonucleotides,
c) vitamins, provitamins or vitamin precursor stages of the groups A, B, C, E, H and K, and the esters of those substances,
d) α-hydroxycarboxylic acids, α-ketocarboxylic acids, β-hydroxycarboxylic acids and their esters, lactones or salt forms,
e) flavonoids and flavonoid-rich plant extracts,
f) isoflavonoids and isoflavonoid-rich plant extracts,
g) polyphenols and polyphenol-rich plant extracts,
h) ubiquinone and ubiquinol and their derivatives,
i) silymarin,
j) ectoin,
k) purine and purine derivatives,
l) monosaccharides, disaccharides, and oligosaccharides,
m) hydantoin and hydantoin derivatives,
n) 2-furanone and 2-furanone derivatives,
o) taurine (2-aminoethanesulfonic acid),
p) carnitine, carnitine tartrate, carnitine magnesium citrate, acetylcarnitine, 3-O-lauroyl-L-carnitine hydrochloride, 3-O-octanoyl-L-carnitine hydrochloride, 3-O-palmitoyl-L-carnitine hydrochloride, taurine, taurine lysylat, taurine tartrate, taurine ornithate, lysyltaurine and ornithyltaurine, betalaine, 1,1-dimethylproline, hercynine (Nα, Nα, Nα-trimethyl-L-histidinium betaine), ergothioneine (thioneine, 2-mercapto-N-α, Nα, Nα-trimethyl-L-histidinium betaine), choline, choline chloride, choline bitartrate, choline dihydrogen citrate, and
q) mixtures of active ingredients a) through p).

37. The hair treatment agent of claim 1 comprising 0.001 to 5% by weight of the copolymer A of
A1) 0.1 to 50% (based on the total number of monomers in the copolymer) monomers having formula (Ia) 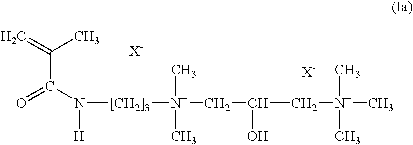
wherein
X represents chloride, sulfate or methosulfate, and
A2) a monomer selected from the group consisting of acrylic acid, methacrylic acid, and the alkali metal and ammonium salts of those acids, wherein the monomer A2 comprises 50 to 99.9% (based on the total number of monomers in the copolymer) of the copolymer.

38. The hair treatment agent of claim 1 comprising 0.001 to 5% by weight of the copolymer A of
A1) 0.1 to 50% (based on the total number of monomers in the copolymer) monomers of formula (Ib) 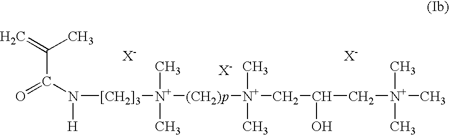
wherein
p represents 2, 3 or 4 and
X represents chloride, sulfate, or methosulfate
A2) a monomer selected from the group consisting of acrylic acid, methacrylic acid, and the alkali metal and ammonium salts of those acids, wherein monomer A2 comprises 50 to 99% (based on the total number of monomers in the copolymer) of the copolymer.

39. The hair treatment agent of claim 1 comprising 0.001 to 5% by weight of the copolymer A having the general formula (Ic) 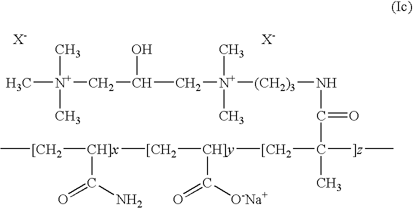
wherein
x+y+z=Q, wherein
Q represents values of 3 to 55,000,
x represents (0 to 0.5) Q,
y represents (0.1 to 0.95) Q, and
z represents (0.001 to 0.5) Q.

40. The hair treatment agent of claim 1 comprising 0.001 to 5% by weight of the copolymer A of the general formula (Id) 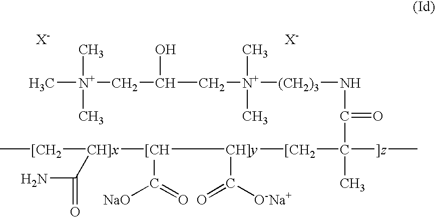
wherein
x+y+z=Q, wherein
Q represents values of 3 to 55,000,
x represents (0 to 0.5) Q,
y represents (0.1 to 0.95) Q, and
z represents (0.001 to 0.5) Q.

41. The hair treatment agent of claim 1 comprising 0.001 to 5% by weight of the copolymer A of the general formula (Ie) 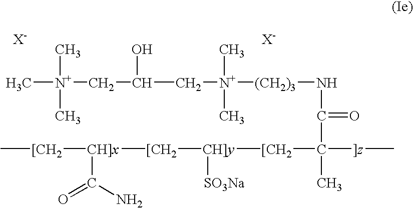
wherein
x+y+z−Q, wherein
Q represents values of 3 to 55,000,
x represents (0 to 0.5) Q,
y represents (0.1 to 0.95) Q, and
z represents (0.001 to 0.5) Q.

42. The hair treatment agent of claim 1 comprising 0.001 to 5% by weight of the copolymer A of the general formula (If) 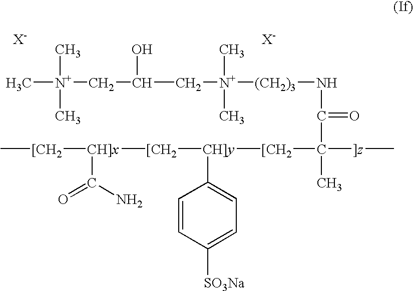
wherein
x+y+z=Q, wherein
Q represents values of 3 to 55,000,
x represents (0 to 0.5) Q,
y represents (0.1 to 0.95) Q, and
z represents (0.001 to 0.5) Q.

43. The hair treatment agent of claim 39, wherein the ratio of (y:z) is 4:1 to 1:2.

44. The hair treatment agent of claim 40, wherein the ratio of (y:z) is 4:1 to 1:2.

45. The hair treatment agent of claim 41, wherein the ratio of (y:z) is 4:1 to 1:2.

46. The hair treatment agent of claim 42, wherein the ratio of (y:z) is 4:1 to 1:2.

47. The hair treatment agent of claim 1, wherein the copolymer A has a molecular weight of 10,000 to 20 million g·mol⁻¹.

48. The hair treatment agent of claim 1, wherein the silicone comprises 0.01 to 5% by weight of formula Si-I
(CH₃)₃Si—[O—Si(CH₃)₂]ₓ—O—Si(CH₃)₃ (Si-I),
wherein x represents a number from 0 to 5,000.

49. The hair treatment agent of claim 1, wherein the silicone comprises 0.01 to 5% by weight of an amino-functional silicone of formula (Si-II)
R′ₐG₃₋ₐ-Si(OSiG₂)ₙ-OSiG_{b}R′_{2-b})ₘ—O—SiG₃₋ₐ-R′ₐ (Si-II),
wherein
G is —H, a phenyl group, —OH, —O—CH₃, —CH₃, —CH₂CH₃, —CH₂CH₂—CH₃, —CH(CH₃)₂, —CH₂CH₂CH₂CH₃, —CH₂CH(CH₃)₂, —CH(CH₃)CH₂CH₃, or —C(CH₃)₃;
a represents a number between 0 and 3;
b represents a number between 0 and 1;
m and n represent numbers for which the sum (m+n) is between 1 and 2,000;
R′ represents a monovalent group selected from the group consisting of
—N(R″)—CH₂—CH₂—N(R″)₂
—N(R″)₂
—N⁺(R″)₃A⁻
—N⁺H(R″)₂A⁻
—N⁺H₂(R″)A⁻
—N(R″)—CH₂—CH₂—N⁺R″H₂A⁻,
wherein each R″ independently is selected from the group consisting of —H, -phenyl, -benzyl-, a C₁₋₂₀ alkyl group; and
A represents an anion.

50. The hair treatment agent of claim 1 comprising, as care substance C, 0.01 to 5% by weight of an amino acid.

51. The hair treatment agent of claim 1 comprising, as care substance C, 0.1 to 5% by weight of a compound selected from the group consisting of vitamins, provitamins, and vitamin precursors.

52. The hair treatment agent of claim 1 comprising, as care substance C, 0.01 to 5% by weight of a carbohydrate selected from the group consisting of monosaccharides, disaccharides and oligosaccharides.

53. A hair treatment agent comprising
A) a copolymer A of
A1) 0.1 to 50% (based on the total number of monomers in the copolymer) monomers having the formula (I) 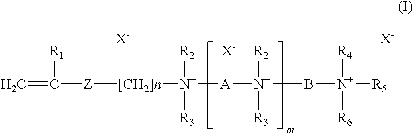
wherein
R¹ represents —H or —CH₃ or —CH₂CH₃
R², R³, R⁴, R⁵, R⁶ independently represent —H or —CH₃ or —CH₂CH₃ or —(C₂)₂CH₃ or —CH(CH₃)₂ or —(CH₂)₃CH₃ or —CH(CH₃)CH₂CH₃ or —CH₂—CH(CH₃)₂ or —C(CH₃)₂ or —CH₂—OH or —CH₂)₂—OH or —CH(OH)—CH₃ or —CH(OH)—CH₂—CH₃ or —CH₂CH(OH)CH₃ or —CH₂)₃OH or —CH₂—NH₂ or —(CH₂)₂—NH₂ or —CH(NH₂)—CH₃ or —CH(NH₂)CH₂CH₃ or —CH₂CH(NH₂)CH₃ or —(CH₂)₃NH₂
n represents values selected from the group consisting of 2, 3 and 4,
m represents values selected from the group consisting of 0, 1 and 2,
Z represents —O— or —C(O)—O— or —C(O)—NH—
A represents —CH₂— or —(CH₂)₂— or —(CH₂)₃— or —(CH₂)₄— or —(CH₂)₅— or —(CH₂)₆—,
B represents a linear or branched C₃-C₆-polymethylene that optionally may be interrupted by a heteroatom selected from the group consisting of —O— and —NH—, and optionally may be substituted with a group selected from hydroxyl and amino, and
X represents an anion selected from the group consisting of chloride, bromide, iodide, sulfate, and methosulfate
A2) represents a monomer selected from the group consisting of acrylic acid, methacrylic acid, alpha-ethylacrylic acid, beta,beta-dimethylacrylic acid, methylenemalonic acid, vinylacetic acid, allylacetic acid, ethylideneacetic acid, propylideneacetic acid, crotonic acid, maleic acid, fumaric acid, itaconic acid, citraconic acid, mesaconic acid, N-methacryloylalanine, N-acryloylhydroxyglycine, sulfopropyl acrylate, sulfoethyl acrylate, sulfoethyl methacrylate, sulfoethyl methacrylate, styrene sulfonic acid, vinylsulfonic acid, vinylphosphonic acid, phosphoethyl acrylate, phosphonoethyl acrylate, phosphopropyl acrylate, phosphonopropyl acrylate, phosphoethyl methacrylate, phosphonoethyl methacrylate, phosphopropyl methacrylate and phosphonopropyl methacrylate, and the alkali metal and ammonium salts of those acids,
A3) represents, optionally, a nonionic monomer selected from the group consisting of acrylamide, vinyl alcohol, C₁-C₄-alkyl esters of acrylic acid, C₁-C₄-alkyl esters of methacrylic acid, C₁-C₄-hydroxyalkyl esters of acrylic acid, C₁-C₄-hydroxyalkyl esters of methacrylic acid, polyalkoxylated esters of acrylic acid, polyalkoxylated esters of methacrylic acid, esters of acrylic acid, esters of methacrylic acid with polyethylene glycol or polypropylene glycol mono-(C₁-C₂₅)-alkyl ethers, vinyl acetate, vinylpyrrolidone and methylvinyl ether, in which the monomers A2 and A3, together, make up 50% to 99.9% (based on the total number of monomers in the copolymer) of the copolymer;
B) a silicone, and
C) a care substance selected from the group consisting of
a) monomers, oligomers and polymers of amino acids, N—(C₂-C₂₄)-acylamino acids, the esters and the physiologically acceptable salts of these substances,
b) DNA or RNA oligonucleotides,
c) vitamins, provitamins or vitamin precursor stages of the groups A, B, C, E, H and K, and the esters of those substances,
d) α-hydroxycarboxylic acids, α-ketocarboxylic acids, β-hydroxycarboxylic acids and their esters, lactones or salt forms,
e) flavonoids and flavonoid-rich plant extracts,
f) isoflavonoids and isoflavonoid-rich plant extracts,
g) polyphenols and polyphenol-rich plant extracts,
h) ubiquinone and ubiquinol and their derivatives,
i) silymarin,
j) ectoin,
k) purine and purine derivatives,
l) monosaccharides, disaccharides, and oligosaccharides,
m) hydantoin and hydantoin derivatives,
n) 2-furanone and 2-furanone derivatives,
o) taurine (2-aminoethanesulfonic acid),
p) carnitine, carnitine tartrate, carnitine magnesium citrate, acetylcarnitine, 3-O-lauroyl-L-carnitine hydrochloride, 3-O-octanoyl-L-carnitine hydrochloride, 3-O-palmitoyl-L-carnitine hydrochloride, taurine, taurine lysylat, taurine tartrate, taurine ornithate, lysyltaurine and ornithyltaurine, betalaine, 1,1-dimethylproline, hercynine (Nα, Nα, Nα-trimethyl-L-histidinium betaine), ergothioneine (thioneine, 2-mercapto-N-α, Nα, Nα-trimethyl-L-histidinium betaine), choline, choline chloride, choline bitartrate, choline dihydrogen citrate, and
q) mixtures of active ingredients a) through p).
